(19) United States
(12) Patent Application Publication
(10) Pub. No.: US 2011/0243846 A1
(43) Pub. Date: Oct. 6, 2011

(54) BENZOTHIAZOLE AMIDES FOR DETECTION OF AMYLOID BETA

(76) Inventors: Jorma Hassfeld, Berlin (DE); Ulrike Roehn, Berlin (DE); Matthias Friebe, Berlin (DE); Lutz Lehmann, Berlin (DE); Tobias Heinrich, Berlin (DE); Sabine Krause, Berlin (DE); Damian Brockschnieder, Berlin (DE); Thomas Dyrks, Berlin (DE); Andrea Thiele, Berlin (DE); Ulf Boemer, Glienicke (DE); Ursula Moenning, Woltersdorf (DE); Markus Berger, Berlin (DE); Stephan Siegel, Berlin (DE)

(21) Appl. No.: 13/139,116

(22) PCT Filed: Nov. 28, 2009
(86) PCT No.: PCT/EP09/08499

§ 371 Date: Jun. 10, 2011

(30) Foreign Application Priority Data
Dec. 12, 2008 (EP) 08075941.8

(51) Int. Cl. A61K 51/04; C07D 277/82; C07F 7/22; C07F 5/02; C07D 417/12; A61P 43/00

(52) U.S. Cl. 424/1.89; 548/163; 548/103; 548/110; 546/270.1; 424/1.65

(57) ABSTRACT

This invention relates to compounds (benzothiazoles) suitable for labelling or already labelled by ¹⁸F, methods of preparing such a compound, compositions comprising such compounds, kits comprising such compounds or compositions and uses of such compounds, compositions or kits for diagnostic imaging.

### FIELD OF INVENTION

This invention relates to compounds suitable for labelling or already labelled by ¹⁸F, methods of preparing such a compound, compositions comprising such compounds, kits comprising such compounds or compositions and uses of such compounds, compositions or kits for diagnostic imaging.

### BACKGROUND ART

Alzheimer's Disease (AD) is a progressive neurodegenerative disorder marked by loss of memory, cognition, and behavioral stability. AD is defined pathologically by extracellular senile plaques comprised of fibrillar deposits of the beta-amyloid peptide (A-beta) and neurofibrillary tangles comprised of paired helical filaments of hyperphosphorylated tau. The 39-43 amino acids comprising A-beta peptides are derived from the larger amyloid precursor protein (APP). In the amyloidogenic pathway, A-beta peptides are cleaved from APP by the sequential proteolysis by beta- and gamma-secretases. A-beta peptides are released as soluble proteins and are detected at low level in the cerebrospinal fluid (CSF) in normal aging brain. During the progress of AD the A-beta peptides aggregate and form amyloid deposits in the parenchyma and vasculature of the brain which can be detected post mortem as diffuse and senile plaques and vascular amyloid during histological examination (for a recent review see: Blennow et al. Lancet. 2006 Jul. 29; 368(9533):387-403). Alzheimers disease (AD) is becoming a great health and social economical problem all over the world. There are great efforts to develop techniques and methods for the early detection and effective treatment of the disease. Currently, diagnosis of AD in an academic memory-disorders clinic setting is approximately 85-90% accurate (Petrella J R et al. Radiology. 2003 226:315-36). It is based on the exclusion of a variety of diseases causing similar symptoms and the careful neurological and psychiatric examination, as well as neuropsychological testing.

Molecular imaging has the potential to detect disease progression or therapeutic effectiveness earlier than most conventional methods in the fields of neurology, oncology and cardiology. Of the several promising molecular imaging technologies having been developed such as optical imaging, MRI, SPECT and PET, PET is of particular interest for drug development because of its high sensitivity and ability to provide quantitative and kinetic data.

For example positron emitting isotopes include carbon, iodine, nitrogen, and oxygen. These isotopes can replace their non-radioactive counterparts in target compounds to produce tracers that function biologically and are chemically identical to the original molecules for PET imaging. Among these isotopes ¹⁸F is the most convenient labelling isotope due to its half life of 111 min which permits the preparation of diagnostic tracers and subsequent study of biochemical processes. In addition, its low β+ energy (634 keV) is also advantageous.

The nucleophilic aromatic and aliphatic [¹⁸F]-fluoro-fluorination reaction is of great importance for [¹⁸F]-fluoro-labelled radiopharmaceuticals which are used as in vivo imaging agents targeting and visualizing diseases, e.g. solid tumours or diseases of brain. A very important technical goal in using [¹⁸F]-fluoro-labelled radiopharmaceuticals is the quick preparation and administration of the radioactive compound due to the fact that the ¹⁸F isotopes have a short half-life of about only 111 minutes.

A couple of methods are known to introduce F-18 e.g. to an aromatic ring (Coenen, Fluorine-18 Labeling Methods: Features and Possibilities of Basic Reactions, (2006), in: Schubiger P. A., Friebe M., Lehmann L., (eds), PET Chemistry—The Driving Force in Molecular Imaging. Springer, Berlin Heidelberg, pp. 15-50). One of the later discoveries is the replacement of an iodonium leaving group with [¹⁸F]fluoride, compare also e.g. WO2005061415(A1), WO2005097713(A1), WO2007010534(A2), WO2007073200(A1) and WO2007141529(A1).

Post-mortem histological examination of the brain is still the only definite diagnosis of this disease. Thus, the in vivo detection of one pathological feature of the disease—the amyloid aggregate deposition in the brain—is thought to have a big impact on the early detection of AD and differentiating it from other forms dementia. Additionally, most disease modifying therapies which are in development are aiming at lowering of the amyloid load in the brain. Thus, imaging the amyloid load in the brain may provide an essential tool for patient stratification and treatment monitoring (for a recent review see: Nordberg. Eur J Nucl Med Mol Imaging. 2008 March; 35 Suppl 1:S46-50).

In addition, amyloid deposits are also known to play a role in amyloidoses, in which amyloid proteins (e.g. tau) are abnormally deposited in different organs and/or tissues, causing disease. For a recent review see Chiti et al. Annu Rev Biochem. 2006; 75:333-66.

Potential ligands for visualizing amyloid aggregates in the brain must show a high binding affinity to amyloid plaques and must cross the blood brain barrier. PET tracers which were already investigated in humans regarding their accumulation in the brain of AD patients are [F-18]FDDNP (1) (Shoghi-Jadid et. al, Am J Geriatr Psychiatry 2002; 10:24-35), [C-11]PIB (2) (Klunk et. al, Ann Neurol. 2004 55:306-319), [C-11]SB-13 (3) (Verhoeff et. al, Am J Geriatr Psychiatry 2004; 12:584-595, BAY94-9172 (4) (Lancet Neurol. (2008), 7(2):114-5.), [C-11]BF227 (Kudo et. al, J Nucl. Med. 2007; 49:554-561), and [F-18]PIB (Farrar et. al Turku PET Symposium, Abstract 49).

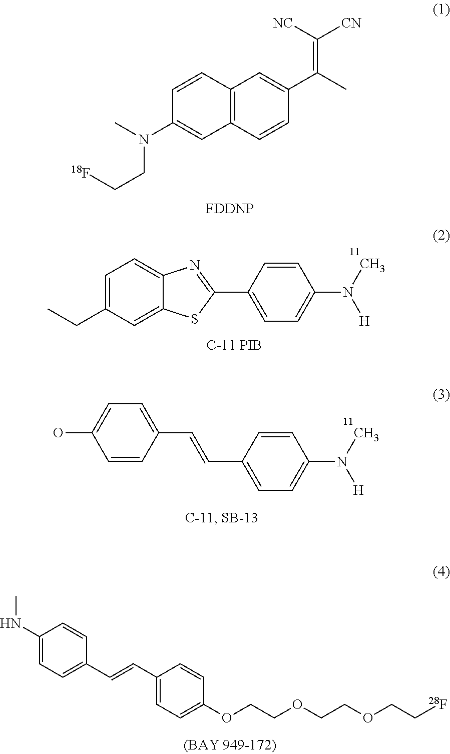

Stilbene derivatives (3 and 4) have been also labelled with PET isotopes and covered by U.S. Pat. No. 7,250,525(B2) and WO2006078384(A2,A3) and members of the corresponding patent families.

It is an important goal for the design of a sufficient CNS-PET tracer that the pharmacokinetics in the brain is optimized. Thus, the PET ligand should enter the brain rapidly in sufficient amount. A high fraction of these molecules should then bind tightly to the target. Subsequently those molecules which have not bound should be eliminated from the surrounding area (“wash-out” from the brain) in order to achieve an image with a high signal to background ratio. Furthermore it is important to have ligands available which show an specific binding to the amyloid plaques.

### (Compare FIG. 1):

The present invention provides novel compounds of Formula I. If these compounds of Formula I are e.g. not ¹⁸F-labelled or ¹⁹F-labelled, but instead contain an appropriate leaving group, they are precursor compounds for the synthesis of ¹⁸F-labelled or ¹⁹F-labelled compounds having formula I. ¹⁹F-labelled compounds having formula I are standard reference compounds (as identification tool and for quality check) of the synthesis towards ¹⁸F-labelled compounds having formula I. In the following compounds of Formula I which contain an appropriate leaving group and do not contain ¹⁸F or ¹⁹F, are also referred to as “precursor compounds having formula I”. Moreover, those compounds of formula I that contain ¹⁹F instead of an appropriate leaving group or a moiety which is suited to be converted to an appropriate leaving group are also referred to as “¹⁹F standard reference compounds having formula I”. Moreover, those compounds of Formula I which contain ¹⁸F and which do not contain an appropriate leaving group or a moiety which is suited to be converted to an appropriate leaving group are also referred to as “¹⁸F-labelled compounds having formula I”. Moreover, those compounds of Formula I which contain a moiety which is suited to be converted to an appropriate leaving group being part of a precursor compound of formula I are also referred to as “starting material having formula I”.
The invention further provides a method of imaging diseases, the method comprising introducing into a patient a detectable quantity of a ¹⁸F-labeled compound of Formula I or a pharmaceutically acceptable salt, ester, amide or prodrug thereof.
The invention provides also ¹⁸F-labelled or ¹⁹F-labelled compounds having formula I for use as medicament.
The present invention also provides diagnostic compositions comprising a radiolabeled compounds preferably ¹⁸F-labelled compounds having formula I, and a pharmaceutically acceptable carrier or diluents.
Another aspect of the invention is directed to the use of compounds of Formula I for the manufacture of a medicament, in particular of ¹⁸F- or ¹⁹-F-labelled compounds having formula I.
The invention also provides a process to synthesize ¹⁸F-labelled compounds having formula I from precursor compounds having formula I.
The invention also provides a process to synthesize ¹⁹F-labelled compounds having formula I from precursor compounds having formula I.
The present invention provides novel compounds of Formula VI. These compounds serve as precursor compounds towards compounds of formula Ib by reacting compounds of Formula IV with compounds of Formula VI. Compounds of formula XIV can be generated by ¹⁸F- or ¹⁹F -fluorinating a compound of formula XV.
The invention also provides a process to synthesize ¹⁸F-labelled compounds having formula Ic by reacting compounds of Formula XIV with compounds of Formula XVI. Compounds of formulae XIV can be generated by ¹⁸F- or ¹⁹F-fluorinating a compound of formula XV.
The invention also provides a kit for preparing a radiopharmaceutical preparation, said kit comprising a sealed vial containing a predetermined quantity of
   a precursor compound having formula I,
   compounds of Formula V and VI
   compounds of Formula XV and XVI.
The invention also provides a process to synthesize “precursor compounds having formula I” (wherein the leaving group of the precursor compound having formula I is attached to a sp²-hybridized carbon atom) from a starting compound having formula I (wherein the chemical functional group which is converted to the leaving group of a precursor compound having formula I is also attached to a sp²-hybridized carbon atom).
The invention also provides a process to synthesize “precursor compounds having formula I” (wherein the leaving group of the precursor compound having formula I is attached to a sp³ hybridized carbon atom) from a starting compound having formula I (wherein the chemical functional group which is converted to the leaving group of a precursor compound having formula I is also attached to a sp³ hybridized carbon atom).
The present invention also provides a kit for imaging diseases. More specifically the compounds of this invention are useful for the imaging of CNS diseases including but not limited to Alzheimer's disease, other forms of dementias (e.g. Lewy body dementia) and/or amyloidoses and/or myelin disorder. The invention, therefore, also relates to the use of imaging compounds for diagnosing these diseases as well as for stratification of therapy and therapy monitoring.
The present invention also relates to a method of imaging amyloid plaques using radioactively labelled compounds of the invention.

### DETAILED DESCRIPTION OF THE INVENTION

In a first aspect the present invention is directed to compounds of formula I

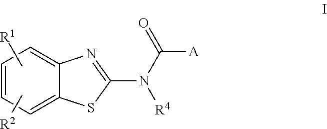

wherein
A is selected from the group comprising 1-(N—R⁹)-2,3-dihydro-1H-indol-5-yl, 1-(N—R⁹)-1H-indol-5-yl, phenyl and pyridyl, whereas A is substituted with R⁵ and R⁶.
R¹ and R² are independently and individually, at each occurrence, selected from the group comprising hydrogen, halo, cyano, trifluoromethyl, (C₁-C₅)alkyl, (C₂-C₅)alkynyl, (C₂-C₅)alkenyl, (C₁-C₅)alkoxy, (R⁷)O—, L-(CH₂—CH₂—O)ₙ—, L, L-(C₁-C₆)alkoxy, (C₁-C₅)sulfanyl and L-(C₁-C₅)sulfanyl;
R⁴ is selected from the group comprising hydrogen and (C₁-C₄)alkyl;
R⁵ and R⁶ are independently and individually, at each occurrence, selected from the group comprising hydrogen, L, L-(C₁-C₅)alkyl, L-(C₂-C₅)alkenyl, L-(C₁-C₅)alkoxy, L-(C₂-C₅)alkynyl, (C₁-C₅)sulfanyl, L-(C₁-C₅)sulfanyl, (C₁-C₅)alkyl, (C₂-C₅)alkenyl, (C₁-C₅)alkoxy, (R⁷)O—, halo, trifluoromethyl, cyano, —C(O)O—((C₁-C₅)alkyl), —N(R⁸)(L-(C₁-C₅)alkyl), —N(L-(C₁-C₄)alkyl)((C₁-C₄)alkyl), —N(R⁸)((C₁-C₄)alkyl) and —N((C₁-C₄)alkyl)₂;
L is selected from the group comprising R¹⁰, R³, [¹⁹F]fluoro and [¹⁸F]fluoro;
R³ is a leaving group;
R¹⁰ is selected from the group comprising R²⁰ and R³⁰;
R²⁰ is selected from the group comprising iodo, —Sn((C₁-C₆)alkyl)₃, —B(OR⁶⁰)(OR⁶¹) and —NMe₂;
R³⁰ is hydroxy;
R⁷ is selected from the group comprising hydrogen and R¹⁷;
R¹⁷ is a phenol-protecting group;
R⁸ is selected from the group comprising hydrogen and R¹⁸;
R¹⁸ is a amine-protecting group;
R⁹ is selected from the group comprising (C₁-C₅)alkyl, L-(C₁-C₅)alkyl and R⁸;
wherein n is an integer from 2 to 6;
including all isomeric forms of said compound, including but not limited to enantiomers and diastereoisomers as well as racemic mixtures,
and any pharmaceutically acceptable salt, ester, amide, complex or prodrug thereof;
with the proviso that compounds of Formula I contain exactly one L.

in a preferred embodiment A is selected from the group comprising 1-(R⁹)-2,3-dihydro-1H-indol-5-yl, phenyl and pyrid-2-yl, whereas A is substituted with R⁵ and R⁶;

In a more preferred embodiment A is selected from the group comprising phenyl and pyrid-2-yl, whereas A is substituted with R⁵ and R⁶;

in one embodiment A is phenyl, whereas A is substituted with R⁵ and R⁶;
in one embodiment A is pyrid-2-yl, whereas A is substituted with R⁵ and R⁶;
in a preferred embodiment R¹ and R² are independently and individually, at each occurrence, selected from the group comprising hydrogen, halo, L, (C₁-C₄)alkyl, (C₁-C₄)alkoxy, (R⁷)O— and L(C₁-C₃)alkoxy;
in a more preferred embodiment R¹ and R² are independently and individually, at each occurrence, selected from the group comprising hydrogen, fluoro, iodo, L, (C₁-C₃)alkyl and (C₁-C₃)alkoxy;
in an even more preferred embodiment R¹ and R² are independently and individually, at each occurrence, selected from the group comprising hydrogen, L, methyl, ethyl and methoxy;
in an even more preferred embodiment R¹ and R² are independently and individually, at each occurrence, selected from the group comprising hydrogen and methoxy;
in another embodiment R¹ and R² are located independently and individually in position 5 and position 6 of the benzothiazol moiety

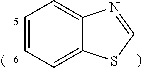

of formula I;
in a preferred embodiment R⁴ is selected from the group comprising hydrogen and methyl;
in a more preferred embodiment R⁴ is hydrogen;
in a preferred embodiment R⁵ and R⁶ are independently and individually, at each occurrence, selected from the group comprising hydrogen, L, L-(C₁-C₄)alkoxy, (C₁-C₄)alkyl, halo, trifluoromethyl, cyano, —N(R⁸)((C₁-C₂)alkyl) and —N((C₁-C₂)alkyl)₂;
in a more preferred embodiment R⁵ and R⁶ are independently and individually, at each occurrence, selected from the group comprising hydrogen, L, L-(C₁-C₃)alkoxy, methyl, bromo, fluoro, trifluoromethyl, cyano, —N(R⁸)(methyl) and —N(methyl)₂;
in an even more preferred embodiment R⁵ and R⁶ are independently and individually, at each occurrence, selected from the group comprising hydrogen, L, methyl, bromo, —N(R⁸)(methyl) and —N(methyl)₂;
in the most preferred embodiment R⁵ and R⁶ are independently and individually, at each occurrence, selected from the group comprising hydrogen and L;
in a preferred embodiment R⁵ and R⁶ are located in para or meta position of the aromatic ring of “A”;
in one embodiment L is [¹⁸F]fluoro; (these compounds are afore mentioned “[¹⁸F]-labelled compounds having formula I”)
in one embodiment L is [¹⁹F]fluoro; (these compounds are afore mentioned “[¹⁹F] standard reference compounds having formula I”)
in one embodiment L is R³; (these compounds are afore mentioned “precursor compounds having formula I”)
in one embodiment L is R¹⁸, (these compounds are afore mentioned “starting compounds having formula I”)
in a preferred embodiment R³ is selected from the group comprising R³³ and R³⁴;
R³³ is selected from the group comprising —I⁺(R²⁵)(X⁻), —I⁺(R²⁶)(X⁻), nitro, —N⁺(Me)₃(X⁻), —S⁺(R²⁵)(R²⁵)(X⁻), —S⁺(R²⁵)(R²⁶)(X⁻), —S⁺(R²⁶)(R²⁶)(X⁻), chloro, and bromo;
R³³ may also be —S(O)₂Me;
in a more preferred embodiment R³³ is selected from the group comprising —I⁺(R²⁵)(X⁻), —I⁺(R²⁶)(X⁻), nitro, —N⁺(Me)₃(X⁻), —S⁺(R²⁵)(R²⁵)(X⁻), —S⁺(R²⁵)(R²⁶)(X⁻), chloro, bromo
in en even more preferred embodiment R³³ is selected from the group comprising —I⁺(R²⁵)(X⁻), —I⁺(R²⁶)(X⁻), nitro, —N⁺(Me)₃(X⁻), —S⁺(R²⁵)(R²⁵)(X⁻), bromo,
in an even more preferred embodiment R³³ is selected from the group comprising —I⁺(R²⁵)(X⁻), —I⁺(R²⁶)(X⁻), nitro, —N⁺(Me)₃(X⁻);
in one embodiment R³³ is selected from the group comprising —I⁺(R²⁵)(X⁻), —I⁺(R²⁶)(X⁻);
in another embodiment R³³ is selected from the group comprising nitro;
in yet another embodiment R³³ is N⁺(Me)₃(X⁻);
in yet another embodiment R³³ is —S(O)₂Me, this embodiment is preferred if R³³ is attached to a pyrid-2-yl moiety;
in one embodiment R³ is R³³, this embodiment is preferred if L and R³ are attached to a sp²-hybridized C-atom;
in one embodiment R³ is R³⁴, this embodiment is preferred if L and R³ is attached to a sp³-hybridized C-atom;
R³⁴ is selected from the group comprising chloro, bromo and iodo, mesyloxy, tosyloxy, trifluormethylsulfonyloxy, nona-fluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutyl-phenyl)sulfonyloxy and (4-methoxy-phenyl)sulfonyloxy;
in a more preferred embodiment R³⁴ is selected from the group comprising bromo, mesyloxy, tosyloxy, (4-nitro-phenyl)sulfonyloxy, (2-nitro-phenyl)sulfonyloxy;
in an even more preferred embodiment R³⁴ is selected from the group comprising mesyloxy, tosyloxy and (4-nitro-phenyl)sulfonyloxy;
R²⁵ is aryl
R²⁶ is heteroaryl
in a preferred embodiment R²⁵ is selected from the group comprising phenyl, (4-methyl)-phenyl, (4-methoxy)-phenyl, (3-methyl)-phenyl, (3-methoxy)-phenyl, (dimethylcarbamoyl)(methyl)amino)phenyl and naphtyl;
in a more preferred embodiment R²⁵ is selected from the group comprising phenyl, (4-methyl)-phenyl and (4-methoxy)-phenyl;
in an even more preferred embodiment R²⁵ is selected from the group comprising phenyl and (4-methoxy)-phenyl;
in one embodiment R²⁵ is (4-(dimethylcarbamoyl)(methyl)amino)phenyl;
in a preferred embodiment R²⁶ is selected from the group comprising 2-furanyl, and 2-thienyl;
in a more preferred embodiment R²⁶ is 2-thienyl.
wherein X⁻ is selected from the group comprising anion of an inorganic acid and anion of an organic acid;
in a preferred embodiment X⁻ is selected from the group comprising CH₃S(O)₂O⁻, CH₃CH₂O⁻, CH₃O⁻, ((4-methyl)phenyl)S(O)₂O⁻, CF₃S(O)₂O⁻, C₄F—₉S(O)₂O⁻, CF₃C(O)O⁻, H₃CC(O)O⁻, iodide anion, bromide anion, chloride anion, perchlorate anion (ClO₄⁻), and phosphate anion;
in a more preferred embodiment X⁻ is selected from the group comprising CF₃S(O)₂O⁻, C₄F₉S(O)₂O⁻, iodide anion, bromide anion and CF₃C(O)O⁻;
in an even more preferred embodiment X⁻ is selected from the group comprising CF₃S(O)₂O⁻, bromide anion and CF₃C(O)O⁻;
in one embodiment X⁻ is selected from the group comprising CH₃CH₂O⁻ and CH₃O⁻;
in another embodiment X⁻ is selected from the group comprising CH₃S(O)₂O⁻, ((4-methyl)phenyl)S(O)₂O⁻, CF₃S(O)₂O⁻, C₄F₉S(O)₂O⁻, CF₃C(O)O⁻, H₃CC(O)O⁻, iodide anion, bromide anion, chloride anion, perchlorate anion (ClO₄⁻), and phosphate anion;
in one embodiment R⁷ is hydrogen;
in another embodiment R⁷ is R¹⁷;
in one embodiment R⁸ is hydrogen;
in another embodiment R⁸ is R¹⁸;
in a preferred embodiment R⁹ is selected from the group comprising (C₁-C₃)alkyl, L-(C₂-C₃)alkyl and R⁸;
in a more preferred embodiment R⁹ is selected from the group comprising methyl and R⁸;

In one embodiment R¹⁰ is R²⁰, this embodiment is preferred if L is attached to a sp²-hybridized C-atom;

In another embodiment R¹⁰ is R³⁰ this embodiment is preferred if L is attached to a sp³-hybridized C-atom;

In a preferred embodiment R²⁰ is selected from the group comprising —Sn((C₁-C₆)alkyl)₃, and —B(OR⁶⁰)(OR⁶¹);

in another embodiment R²⁰ is —NMe₂;
in yet another embodiment R²⁰ is iodo;
R⁶⁰ and R⁶¹ are independently and individually selected from the group comprising hydrogen, (C₁-C₆)alkyl and cycloalkyl, whereas R⁶⁰ and R⁶¹ can be liked to each other by a methylen “bridge”;
in a preferred embodiment R¹⁷ is selected from the group comprising ethoxy-methyl, methoxy-methyl, 2-methoxyethoxymethyl, methylthiomethyl, cyclohexyl, tert butyl, benzyl, (H₃C—)C(O)—, (CH₃O—)C(O)—, (H₃C—CH₂—O—)C(O)—, (benzyl-O—) C(O)— and (phenyl-)C(O)—;
in a more preferred embodiment R¹⁷ is selected from the group comprising ethoxy-methyl, tert butyl, H₃C—C(O)— and H₃C—CH₂—O—C(O)—;
in an even more preferred embodiment R¹⁷ is selected from the group comprising ethoxy-methyl and H₃C—C(O)—;
in a preferred embodiment R¹⁸ is selected from the group comprising (tert-butoxy)-carbonyl, triphenylmethyl, ((para-methoxy)phenyl-diphenyl)methyl, (1-adamantyloxy)carbonyl, (diphenylmethoxy)carbonyl, (cinnamoyloxy)carbonyl, (cyclobutyloxy)carbonyl, ((1-methyl)cyclobutyloxy)carbonyl, ((1-methyl-1-phenyl)ethyloxy)carbonyl, ((1-methyl-1-(4-biphenylyl))ethyloxy)carbonyl, (vinyloxy)carbonyl, formyl, pivaloyloxymethyl and diphenylphosphinyl;
in a more preferred embodiment R¹⁸ is selected from the group comprising (tert-butoxy)-carbonyl, triphenylmethyl, (diphenylmethoxy)carbonyl, ((1-methyl-1a phenyl)ethoxy)carbonyl and formyl;
in an even more preferred embodiment R¹⁸ is selected from the group comprising (tert-butoxy)-carbonyl and formyl:
in a preferred embodiment n is an integer from 2 to 5;
in a more preferred embodiment n is an integer from 2 to 4;
in an even more preferred embodiment n is an integer from 2 to 3.

In one embodiment of general formula I, L is R¹⁰; these are the aforementioned “starting compounds”;

in preferred starting compounds having formula I R⁸ is R¹⁸ as defined above;
and R⁷ is R¹⁷ as defined above;
preferred “starting compounds having formula I” are

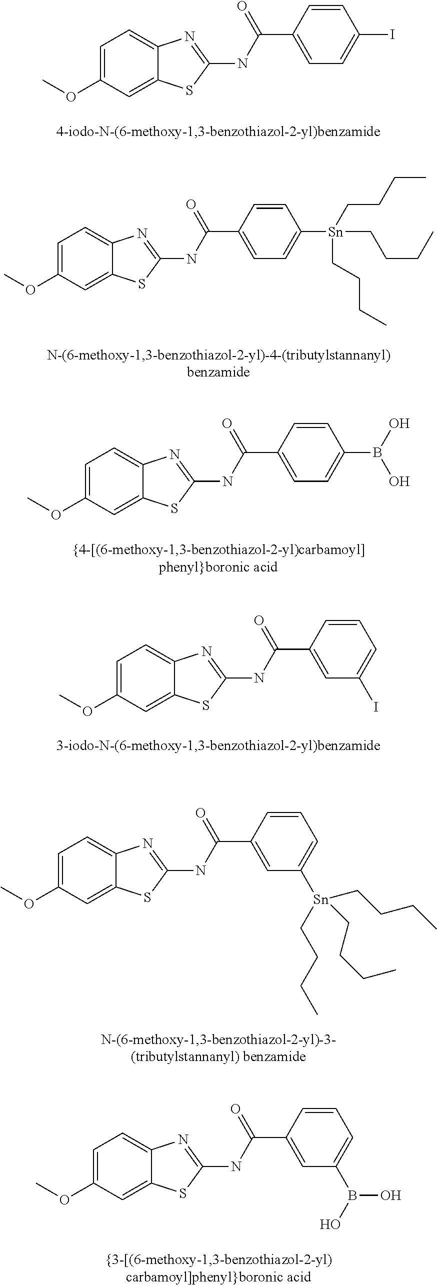

In one embodiment of general formula I, L is R³; these are the aforementioned “precursor compounds having formula I”.

in preferred precursor compounds having formula I R⁷ is R¹⁷;
in preferred precursor compounds having formula I R⁸ is R¹⁸;
preferred “precursor compounds having formula I” are

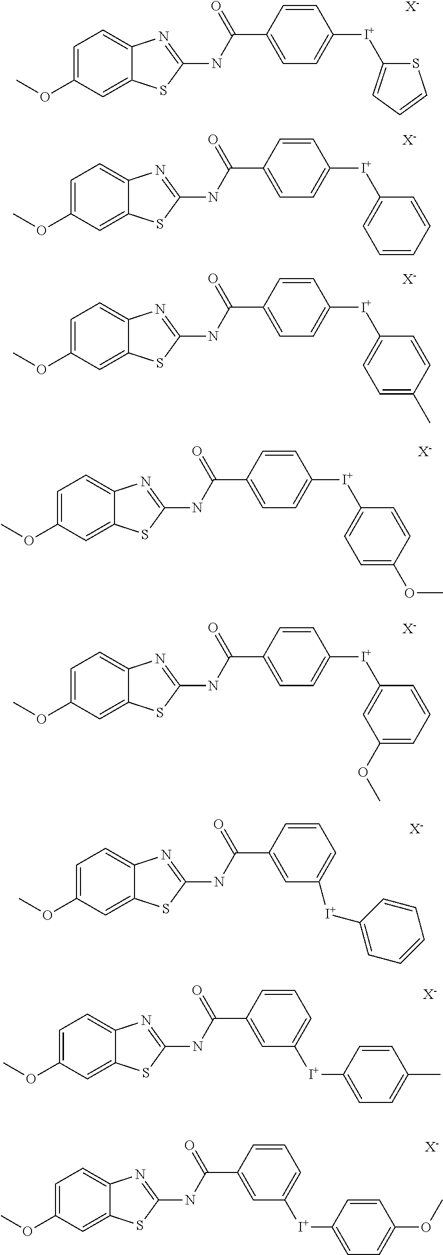

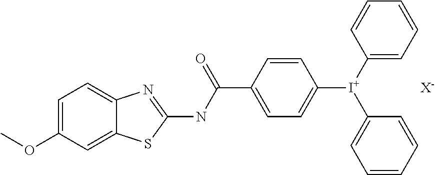

wherein X⁻ is defined as above;

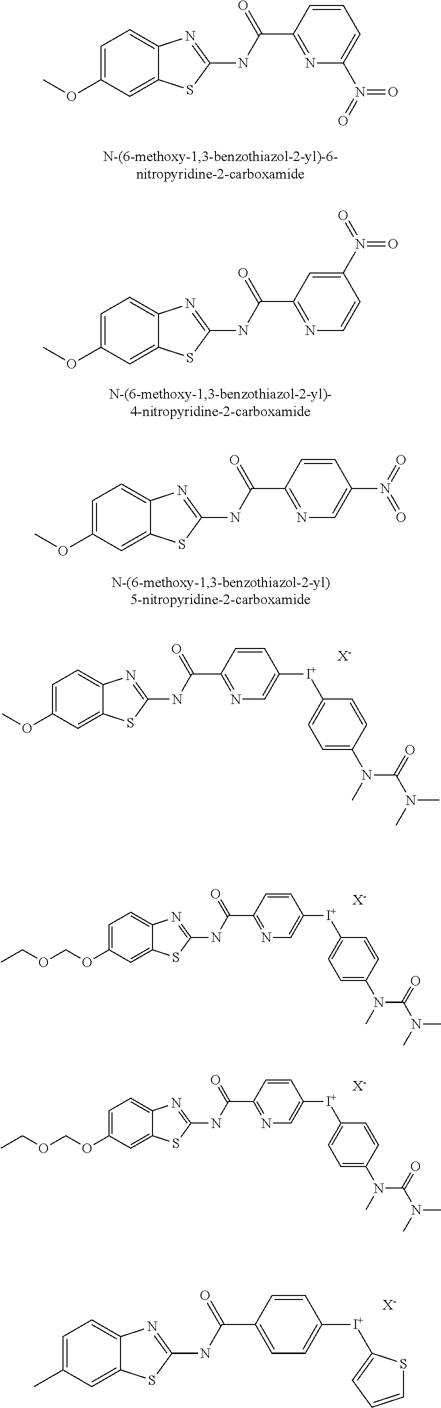

wherein X⁻ is selected from the group comprising anion of an inorganic acid and anion of an organic acid;
more preferred “precursor compounds having formula I” are

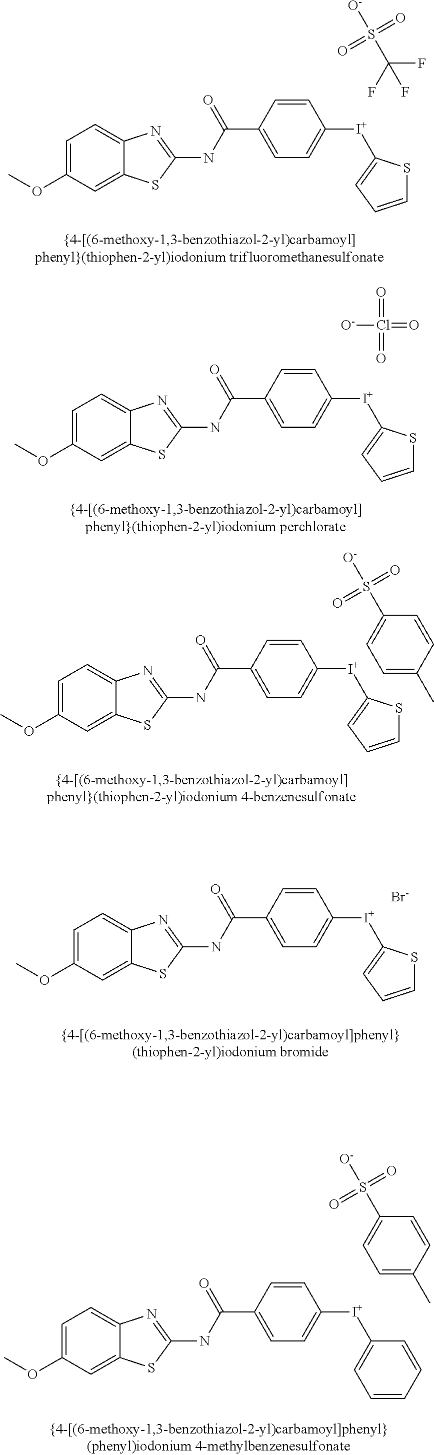

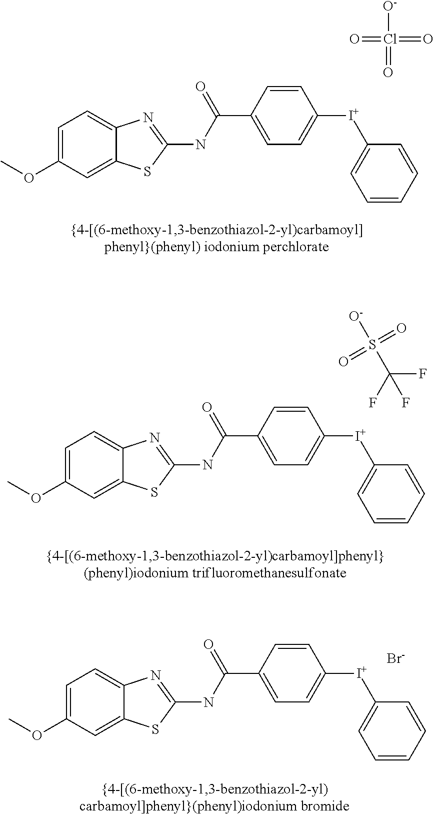

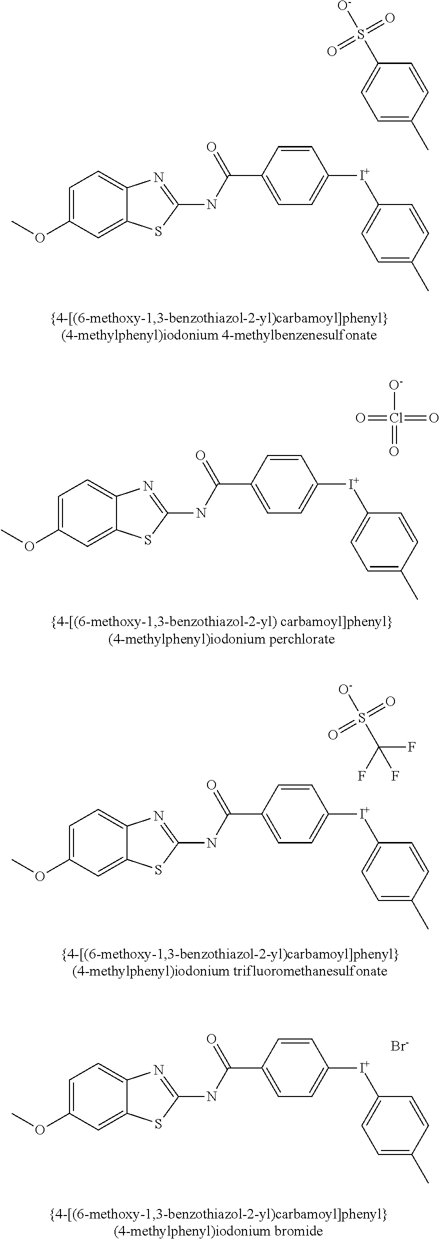

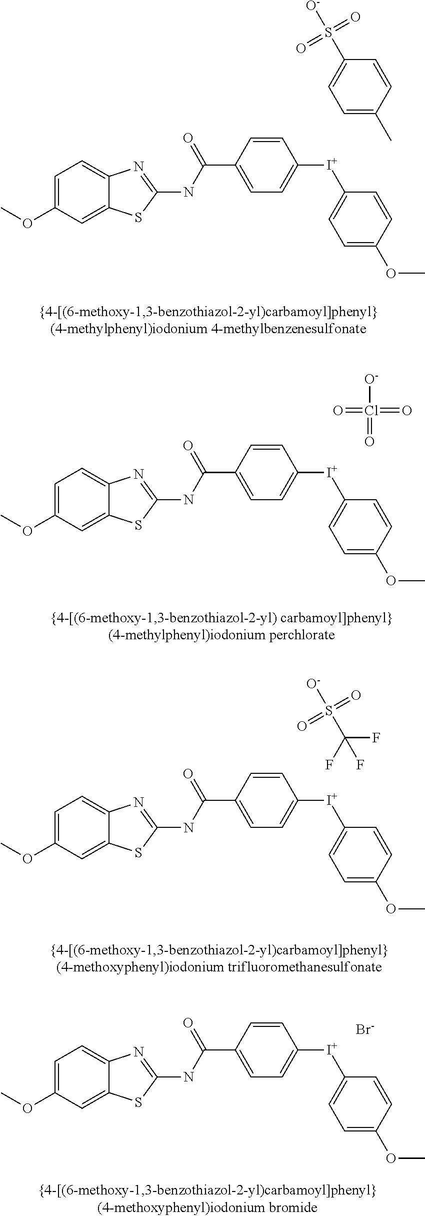

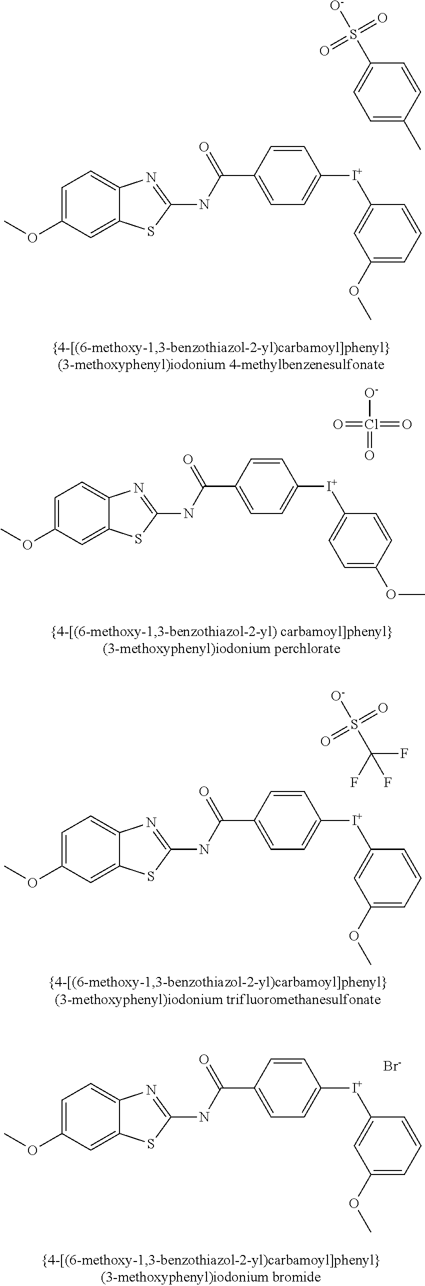

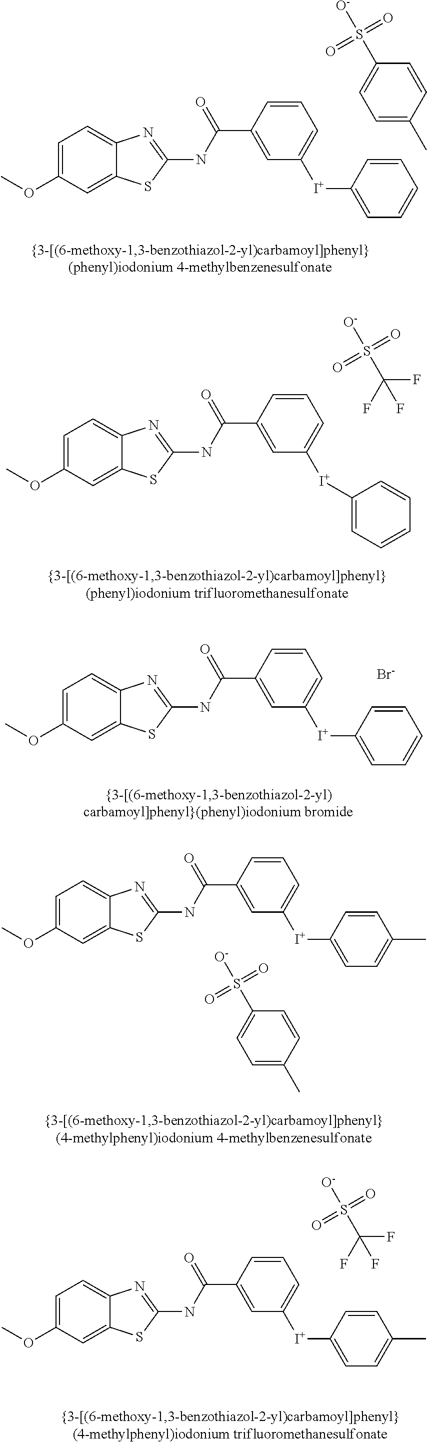

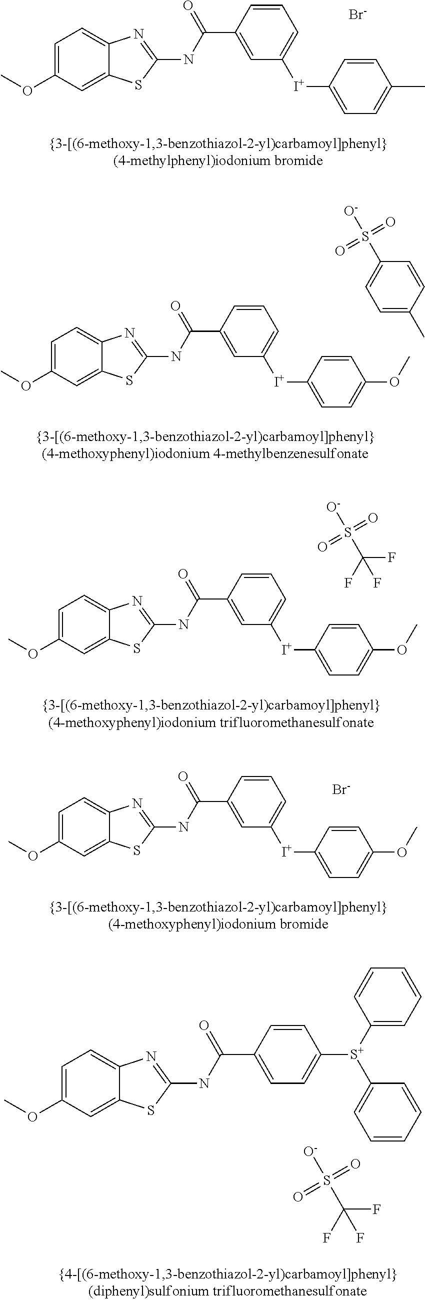

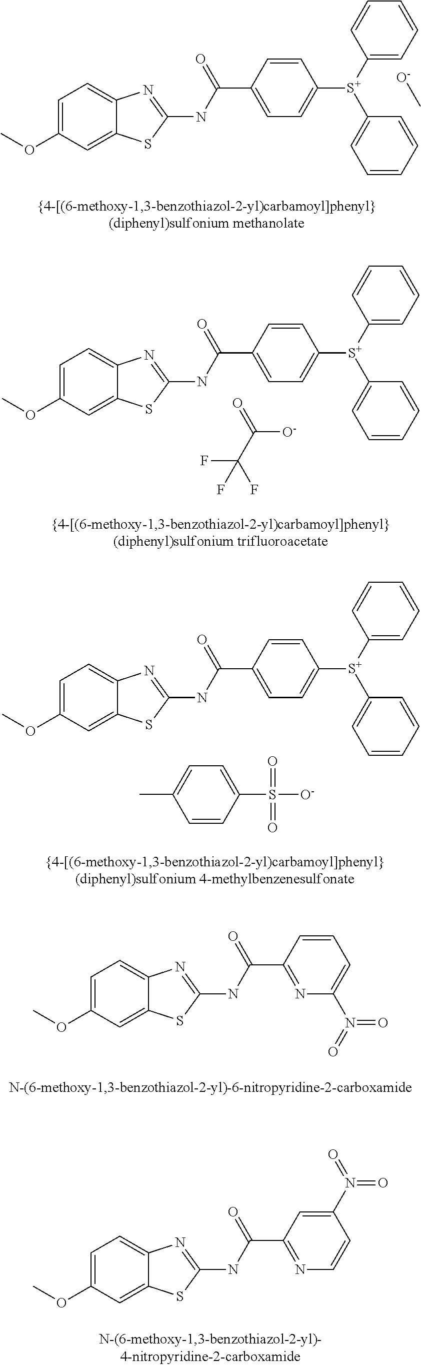

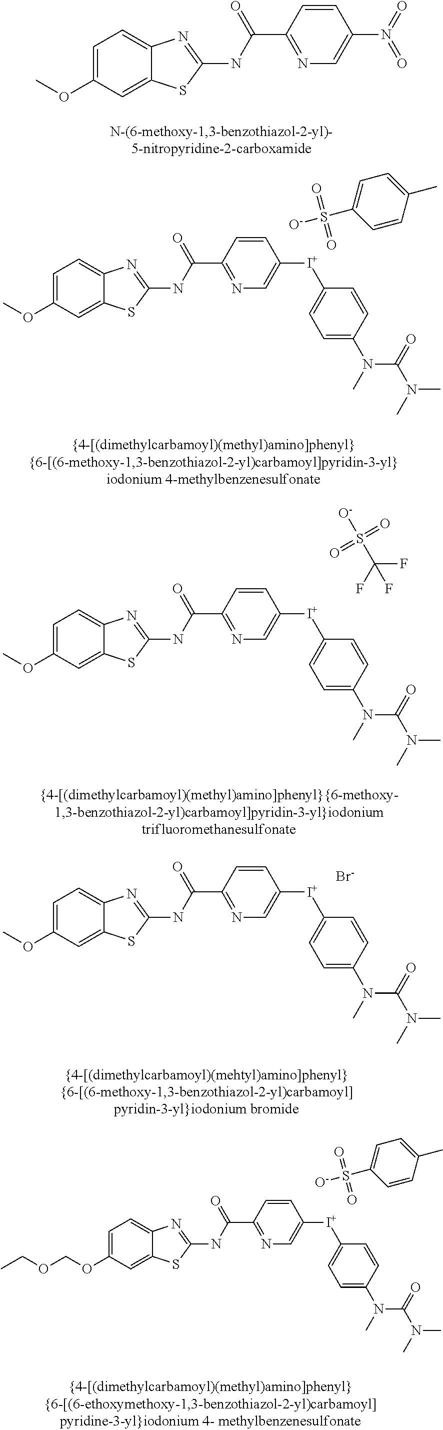

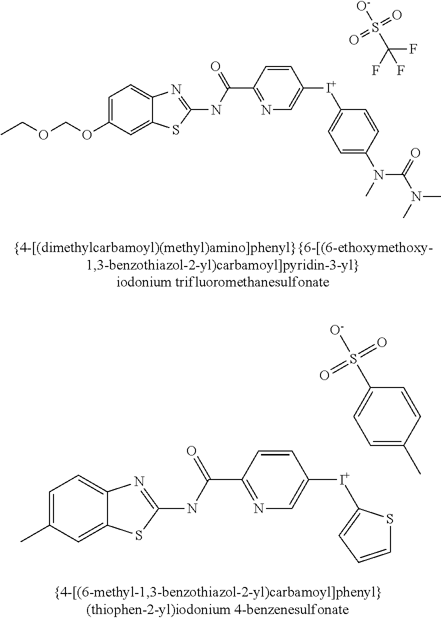

In another embodiment of general formula I, L is [¹⁸F]fluoro, these are the ¹⁸F-labelled compounds having formula I.

Preferred “F-18 labelled compounds having formula I” are

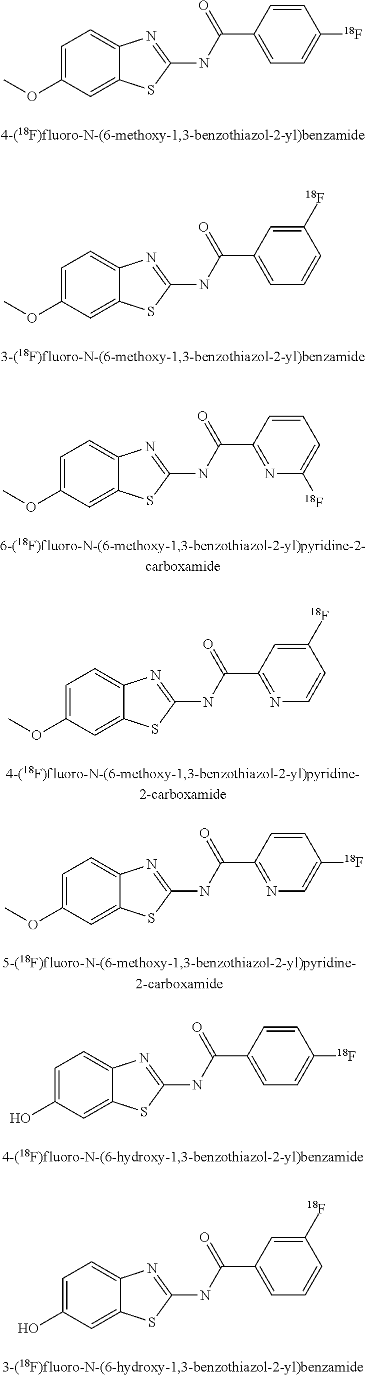

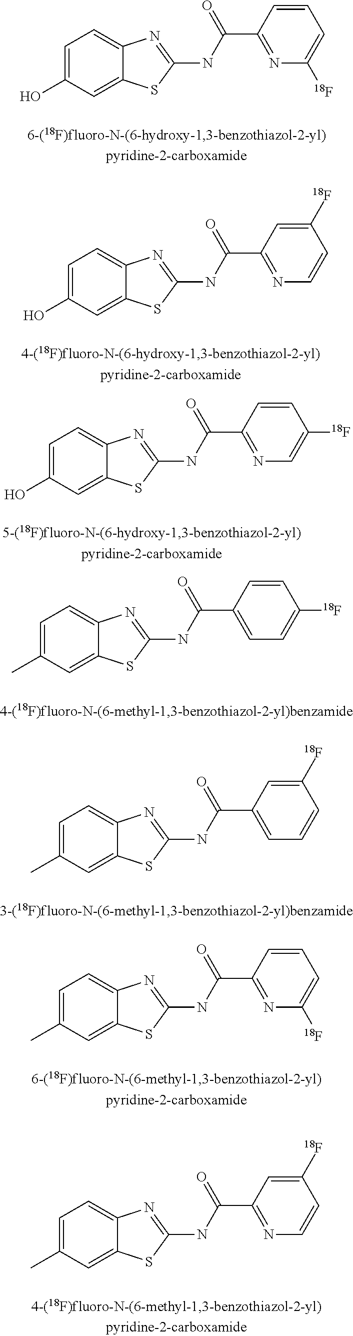

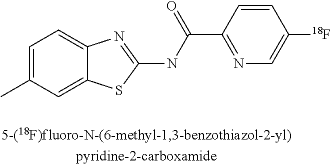

In yet another embodiment of general formula I, L is [¹⁹F]fluoro, these are the aforementioned “standard reference compounds” having formula I.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference;

The term “phenol-protecting group” as employed herein by itself or as part of another group is known or obvious to someone skilled in the art, which is chosen from but not limited to a class of protecting groups namely ethers, esters, carbonates, phosphinates, sulfonates, acetals and ketals and which is chosen from but not limited to those described in the textbook Greene and Wuts, Protecting groups in Organic Synthesis, third edition, page 249-290, included herewith by reference;

The term “anion of inorganic or organic acids” as employed herein refers to the corresponding base of mineral acids, including but not limited to: acids such as carbonic, nitric or sulphuric acid, hydrogen chloride, hydrogen bromide, hydrogen iodide, phosphoric acid, perchloric acid or to the corresponding base of appropriate organic acids which includes but not limited to: alkanols ((C₁-C₁₀)alkyl alcohol), acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic and sulfanilic acid; it is obvious to someone skilled in the art that these so-called organic acids can be substituted by one or more appropriate substituents, such as OH, halo, (C₁-C₆)alkyl, CF₃, CN, (C₁-C₆)alkenyl, (C₁-C₆)alkynyl, (C₁-C₆)alkoxy, (dimethylcarbamoyl)(methyl)amino, NH₂, NO₂, SO₃H, —SO₂NH₂, —N(H)C(O)(C₁-C₅)alkyl, C(O)N(H)(C₁-C₅)alkyl, perfluoro-alkyl chains etc. and combinations of them so that each substituent is substituted by another one.

The term “leaving group” as employed herein by itself or as part of another group is known or obvious to someone skilled in the art, and means that an atom or group of atoms is detachable from a chemical substance by a nucleophilic agent, eg. fluoride atom. Typically the leaving group is displaced as stable species taking with it the bonding electrons.

R³ is a leaving group which is known or obvious to someone skilled in the art and which is taken from but not limited to those described or named in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O— nonaflat”); Carey and Sundberg, Organische Synthese, (1995), page 279-281, table 5.8; is Netscher, Recent Res. Dev. Org. Chem., 2003, 7, 71-83, scheme 1, 2, 10 and 15 and others); Journal of Fluorine Chemistry, 80, 2, (1996), 163-166 (sulphonium as electrophile); Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33).

It should be clear that wherever in this description the terms “aryl”, “heteroaryl” or any other term referring to an aromatic system is used, this also includes the possibility that such aromatic system is substituted by one or more appropriate substituents, such as OH, halo, (C₁-C₆)alkyl, CF₃, CN, (C₁-C₆)alkenyl, (C₁-C₆)alkynyl, (C₁-C₆)alkoxy, (dimethylcarbamoyl)(methyl)amino, NH₂, NO₂, SO₃H, —SO₂NH₂, —N(H)C(O)(C₁-C₅)alkyl, —C(O)N(H)(C₁-C₅)alkyl etc.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl, which themselves can be substituted with one, two or three substituents independently and individually selected from the group comprising halo, nitro, (C₁-C₆)carbonyl, cyano, nitrite, hydroxyl, perfluoro-(C₁-C₁₆)alkyl, in particular trifluormethyl, (C₁-C₆)alkylsulfonyl, (C₁-C₆)alkyl, (C₁-C₆)alkoxy, (dimethylcarbamoyl)(methyl)amino and (C₁-C₆)alkylsulfanyl. As outlined above such “aryl” may additionally be substituted by one or several substituents. It is obvious to someone skilled in the art that afore mentioned substituents can be also combined within one and the same substituents (e.g. halo-alkyl, perfluoroalkyl-alkoxy, ed.)

The term “heteroaryl” as employed herein refers to groups having 5 to 14 ring atoms; 6, 10 or 14 π (pi) electrons shared in a cyclic array; and containing carbon atoms (which can be substituted with halo, nitro, ((C₁-C₆)alkyl)carbonyl, cyano, hydroxyl, trifluormethyl, (C₁-C₆)sulfonyl, (C₁-C₆)alkyl, (C₁-C₆)alkenyl, (C₁-C₆)alkynyl, (C₁-C₆)alkoxy or ((C₁-C₆)alkyl)sulfanyl and 1, 2, 3 or 4 oxygen, nitrogen or sulfur heteroatoms (where examples of heteroaryl groups are: thienyl, benzo[b]thienyl, naphtho[2,3-b]thienyl, thianthrenyl, (uranyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl and phenoxazinyl groups).

As outlined above such “heteroaryl” may additionally be substituted by one or several substituents.

As used hereinafter in the description of the invention and in the claims, the term “alkyl”, by itself or as part of another group, refers to a straight chain or branched chain alkyl group with 1 to 10 carbon atoms such as, for example methyl, ethyl, propyl, isopropyl, butyl, isobutyl, tert-butyl, pentyl, isopentyl, neopentyl, heptyl, hexyl, decyl. Alkyl groups can also be substituted, such as by halogen atoms, hydroxyl groups, C₁-C₄ alkoxy groups or C₆-C₁₂ aryl groups (which, in turn, can also be substituted, such as by 1 to 3 halogen atoms). More preferably alkyl is (C₁-C₁₀)alkyl, (C₁-C₆)alkyl or (C₁-C₄)alkyl.

As used hereinafter in the description of the invention and in the claims, the term “alkenyl” and “alkynyl” is similarly defined as for alkyl, but contain at least one carbon-carbon double or triple bond, respectively.

As used hereinafter in the description of the invention and in the claims, the term “alkoxy (or alkyloxy)” refer to alkyl groups respectively linked by an oxygen atom, with the alkyl portion being as defined above.

As used herein in the description of the invention and in the claims, the substituent L as defined above and being part of the substituents “alkyl”, “alkenyl”, “alkynyl”, “alkoxy” ect. can be attached at any carbon of the corresponding substituent “alkyl”, “alkenyl”, “alkynyl, “alkoxy” ect. Thus, e.g. the term “L-(C₁-C₅)alkoxy” does include different possibilities regarding positional isomerism, e.g. L-(C₅)pentoxy can mean e.g. L-CH₂—CH₂—CH₂—CH₂—CH₂—O—, CH₃—C(L)H—CH₂—CH₂—CH₂—O— or CH(—CH₂-L)(—CH₃)—CH₂—CH₂—O—, ect.

Whenever the term “substituted” is used, it is meant to indicate that one or more hydrogens attached to the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a pharmaceutical composition. The substituent groups may be selected from halogen atoms (fluoro, chloro, bromo, iodo), hydroxyl groups, —SO₃H, nitro, (C₁-C₆)alkylcarbonyl, cyano, nitrile, trifluoromethyl, (C₁-C₆)alkylsulfonyl, (C₁-C₆)alkyl, (C₂-C₆)alkenyl, (C₁-C₆)alkynyl, (C₁-C₆)alkoxy and (C₁-C₆)alkylsulfanyl.

The term “halo” refers to fluorine (F), chlorine (Cl), bromine (Br), and iodine (I). If a chiral center or another form of an isomeric center is present in a compound according to the present invention, all forms of such stereoisomer, including enantiomers and diastereoisomers, are intended to be covered herein. Compounds containing a chiral center may be used as racemic mixture or as an enantiomerically enriched mixture or the racemic mixture may be separated using well-known techniques and an individual enantiomer maybe used alone. In cases in which compounds have unsaturated carbon-carbon bonds double bonds, both the (Z)-isomer and (E)-isomers are within the scope of this invention. In cases wherein compounds may exist in tautomeric forms, such as keto-enol tautomers, each tautomeric form is contemplated as being included within this invention whether existing in equilibrium or predominantly in one form.

Unless otherwise specified, when referring to the compounds of formula the present invention per se as well as to any pharmaceutical composition thereof the present invention includes all of the hydrates, salts, solvates, complexes, and prodrugs of the compounds of the invention. Prodrugs are any covalently bonded compounds, which releases the active parent pharmaceutical according to formula I.

As used hereinafter in the description of the invention and in the claims, the terms “inorganic acid” and “organic acid”, refer to mineral acids, including, but not being limited to: acids such as carbonic, nitric, hydro chloric, hydro bromic, hydro iodic, phosphoric acid, perchloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic, trifluormethansulfonic and sulfanilic acid, respectively.

In a second aspect of the invention the ¹⁸F-labelled compounds having formula I, and the ¹⁹F standard reference compounds having formula I are provided as a medicament or pharmaceutical.

The invention relates also to the use of the ¹⁸F-labelled compounds having formula I, and of the ¹⁹F standard reference compounds having formula I for the manufacture of a medicament or a pharmaceutical for treatment.

In a more preferred embodiment the use concerns the treatment of a CNS disease. CNS diseases include but are not limited to dementias, neurodegenerative diseases and amyloidoses.

More preferably, the CNS disease is selected from multiple sclerosis, Alzheimer's disease, myelin disorder, frontotemporal dementia, dementia with Lewy bodies, amyotrophic lateral sclerosis, Parkinson's Disease, encephalopathies.

The present invention is also directed to a method of treatment or prevention of a disease of the central nervous system, as defined above, comprising the step of introducing into a patient a suitable quantity of a compound of formula I, preferably an ¹⁸F-labelled compound of formula I, or of a ¹⁹F standard reference compound of formula I.

In a third aspect of the invention, ¹⁸F-labelled compounds having formula I are provided as diagnostic imaging agent or imaging agent, preferably as imaging agent for PET applications. It is obvious to persons skilled in the art that compounds of formula I and related derivatives, e.g. compounds of formula I wherein L is iodo and is attached to a sp²-hybridized carbon-atom of formula I are suited as imaging agents for SPECT applications (e.g. L=I-123) or PET-applications (L=I-124).

Furthermore, it is obvious to persons skilled in the art that compounds of formula I and related derivatives, e.g. compounds of formula I wherein L is selected from the group ¹¹CH₃, —O(¹¹CH₃), —N(¹¹CH₃)(C₁-C₅)alkyl, ect. and is preferably attached to a sp²-hybridized carbon-atom of formula I are suited as imaging agents for PET-applications.

In preferred [¹⁸F]-labelled compounds having formula I which are provided as imaging agents R⁷ is hydrogen and R⁸ is hydrogen.

The invention relates also to the use of ¹⁸F-labelled compounds having formula I for the manufacture of an imaging agent.

In a more preferred embodiment the use concerns the imaging of CNS diseases. CNS diseases include but are not limited to Alzheimer's disease, frontotemporal dementia, dementia with Levy bodies, myelin disorder, diseases of unclear origin.

The present invention is also directed to a method of imaging comprising the step of introducing into a patient a detectable quantity of an ¹⁸F-labelled compound of formula I and imaging said patient.

The compounds as described above and herein are, in a preferred embodiment of the invention, bound to an Aβ peptide.

The compounds as described above and herein are, in a preferred embodiment of the invention, bound to a tau filament or tangle.

Another aspect of the invention is the use of a compound of formula I as described above and herein for diagnosing and/or treating Alzheimer's disease and/or amyloidoses in a patient, in particular in a mammal, such as a human.

The treatment of a patient with Alzheimer's disease and/or amyloidoses can preferably be performed with a compound of the invention according to formula I that does not bear a radioactive label, but in which L is e.g. hydrogen.

Preferably, the use of a compound of the invention in the diagnosis is performed using positron emission tomography (PET), single photon emission computed tomography (SPECT), magnetic resonance (MR)-spectoscropy or tomography.

Another aspect of the invention is directed to a method of imaging amyloid deposits. Such a method comprises a) administering to a mammal a compound as described above and herein containing a detectable label, and b) detecting the signal stemming from the compound that is specifically bound to the amyloid deposits. The specific binding is a result of the high binding affinity of the compounds of the present invention to the amyloid deposits.

In a further aspect, the invention is directed to a method of diagnosing a patient with Alzheimer's disease or amyloidoses. This method comprises a) administering to a human in need of such diagnosis a compound of the invention with a detectable label for detecting the compound in the human as described above and herein, and b) measuring the signal from the detectable label arising from the administration of the compound to the human, preferably by using a gamma camera, by positron emission tomography (PET), or by single photon emission computed tomography (SPECT).

A further embodiment of the invention includes a diagnostic method for other neurological disorders as Alzheimer's disease comprising the exclusion of Alzheimer's disease in a patient, that method comprising administering a compound of the invention to a patient and applying an imaging method of the invention.

A further aspect of the invention refers to a diagnostic composition for imaging amyloid deposits, comprising a radiolabeled compound according to formula I.

The diagnostic methods of the invention can also be used as post-mortem diagnostic methods.

Furthermore, the diagnostic methods of the invention can also be used for monitoring the therapy of Alzheimer's disease, a neurodegenerative disorder or an amyloidoses.

Furthermore, the diagnostic methods of the invention can also be used in diagnosing neurological disorders other than Alzheimer's disease by excluding Alzheimer's disease.

In a further aspect of the invention, the invention comprises a method of treating or preventing amyloidoses or Alzheimer's disease comprises administering to a human in need of such a treatment a compound of formula I as described herein.

A further aspect of the invention refers to a pharmaceutical composition which comprises a compound of the invention as described herein, optionally together with a suitable carrier and/or additive.

Furthermore, the compounds of the invention can also be used as tools in screening, for example high throughput screening methods and in vitro assays. Yet another aspect of the invention refers to a method of inhibiting the formation of amyloid or modulating the pathogenicity of amyloid in a mammal. This method comprises administering a compound of formula I as described herein in an amount that is effective to inhibit the formation of amyloid or to modulate the pathogenicity of amyloid.

The invention also refers to a method for synthesizing a compound of the invention according to formula I as described herein. The general synthetic methods of the compounds of the invention are as follows.

It has been surprisingly found out that compounds of formula I show not only a good binding to Aβ amyloid (compare FIGS. 2, 4, 6 and 7) but also an improved pharmacokinetics regarding high brain uptake in mice at early time points and an accelerated elimination from mouse brain at later time points (compare FIGS. 1, 3 and 5) characterized by a high value of the 2 min/30 min ratio of the percentages of injected dose per gram tissue (% ID/g) (compare FIG. 8). Additionally, preferred compounds of formula I are surprisingly synthesized in a shorter chemical route compared to compounds mentioned in e.g. WO2007/086800.

In a fourth aspect of the invention, pharmaceutical compositions are provided comprising a compound according to formula I, preferably ¹⁸F-labelled compounds having formula I, or ¹⁹F standard reference compounds having formula I or a pharmaceutically acceptable salt of an inorganic or organic acid thereof, a hydrate, a complex, an ester, an amide, a solvate or a prodrug thereof. Preferably the pharmaceutical composition comprises a physiologically acceptable carrier, diluent, adjuvant or excipient.

In a preferred embodiment, pharmaceutical compositions according to the present invention comprise a compound of formula I that is a pharmaceutical acceptable salt, hydrate, complex, ester, amide, solvate or a prodrug thereof.

In a fifth aspect of the invention, a radiopharmaceutical composition is provided comprising an ¹⁸F-labelled compound of formula I or a pharmaceutically acceptable salt of an inorganic or organic acid thereof, a hydrate, a complex, an ester, an amide, a solvate or a prodrug thereof.

Preferably the pharmaceutical composition comprises a physiologically acceptable carrier, diluent, adjuvant or excipient.

The compounds according to the present invention, preferably the radioactively labelled compounds according to Formula I provided by the invention may be administered intravenously in any pharmaceutically acceptable carrier, e.g. conventional medium such as an aqueous saline medium, or in blood plasma medium, as a pharmaceutical composition for intravenous injection. Such medium may also contain conventional pharmaceutical materials such as, for example, pharmaceutically acceptable salts to adjust the osmotic pressure, buffers, preservatives and the like. Among the preferred media are normal saline solution and plasma.

Suitable pharmaceutical acceptable carriers are known to someone skilled in the art. In this regard reference can be made to e.g. Remington's Practice of Pharmacy, 13th ed. and in J. of. Pharmaceutical Science & Technology, Vol. 52, No. 5, September-October, p. 238-311, included herein by reference.

The concentration of the compounds of formula I, preferably of the ¹⁸F-labelled compound according to the present invention and the pharmaceutically acceptable carrier, for example, in an aqueous medium, varies with the particular field of use. A sufficient amount is present in the pharmaceutically acceptable carrier when satisfactory visualization of the imaging target (e.g. a tumor) is achievable.

The compounds according to the present invention, in particular the ¹⁸F-radioactively labelled compounds according to the present invention, i.e. the ¹⁸F-labelled compounds having formula I, provided by the invention may be administered intravenously in any pharmaceutically acceptable carrier, e.g., conventional medium such as an aqueous saline medium, or in blood plasma medium, as a pharmaceutical composition for intravenous injection. Such medium may also contain conventional pharmaceutical materials such as, for example, pharmaceutically acceptable salts to adjust the osmotic pressure, buffers, preservatives and the like. Among the preferred media are normal saline and plasma. Suitable pharmaceutical acceptable carriers are known to the person skilled in the art. In this regard reference can be made to e.g., Remington's Practice of Pharmacy, 11th ed. and in J. of. Pharmaceutical Science & Technology, Vol. 52, No. 5, September-October, p. 238-311.x

In accordance with the invention, the radiolabelled compounds having general chemical Formula I either as a neutral composition or as a salt with a pharmaceutically acceptable counter-ion are administered in a single unit injectable dose. Any of the common carriers known to those with skill in the art, such as sterile saline solution or plasma, can be utilized after radiolabelling for preparing the injectable solution to diagnostically image various organs, tumors and the like in accordance with the invention. Generally, the unit dose to be administered for a diagnostic agent has a radioactivity of about 0.1 mCi to about 100 mCi, preferably 1 mCi to 20 mCi. For a radiotherapeutic agent, the radioactivity of the therapeutic unit dose is about 10 mCi to 700 mCi, preferably 50 mCi to 400 mCi. The solution to be injected at unit dosage is from about 0.01 ml to about 30 ml. For diagnostic purposes after intravenous administration, imaging of the organ or disease in vivo can take place in a matter of a few minutes. However, imaging takes place, if desired, in hours or even longer, after injecting into patients. In most instances, a sufficient amount of the administered dose will accumulate in the area to be imaged within about 0.1 of an hour to permit the taking of scintigraphic images. Any conventional method of scintigraphic imaging for diagnostic purposes can be utilized in accordance with this invention.

As used hereinafter in the description of the invention and in the claims, the term “prodrug” means any covalently bonded compound, which releases the active parent pharmaceutical according to formula I, preferably the ¹⁸F labelled compound of formula I.

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmaco-logical Basis of Therapeutics, 8 ed, McGraw-HiM, Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxy group, such as the hydroxy group on the asymmetric carbon atom, or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference.

Prodrugs can be characterized by excellent aqueous solubility, increased bioavailability and are readily metabolized into the active inhibitors in vivo.

In a sixth aspect the present invention is directed to compounds of Formula wherein L is [¹⁹F]fluoro,

preferred compounds (“standard reference compounds”) of formula I, with L being [¹⁹F]fluoro are:

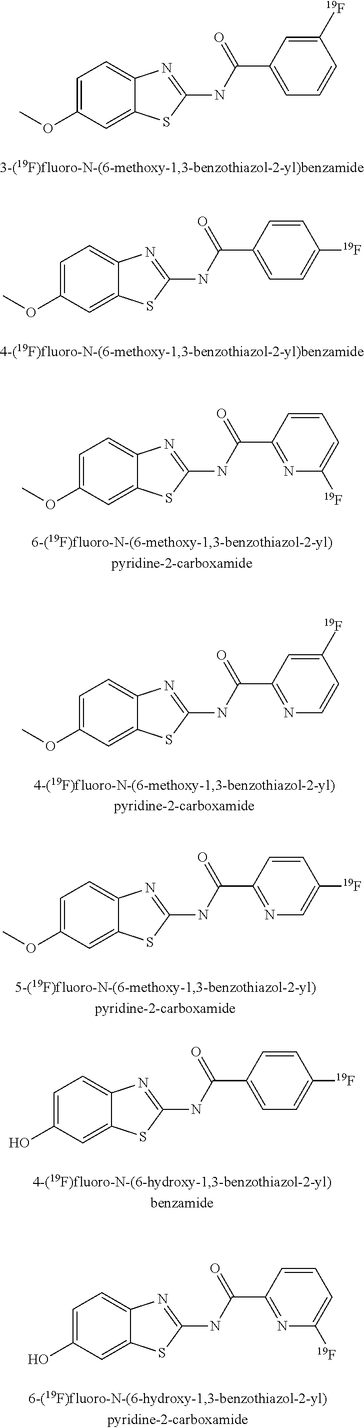

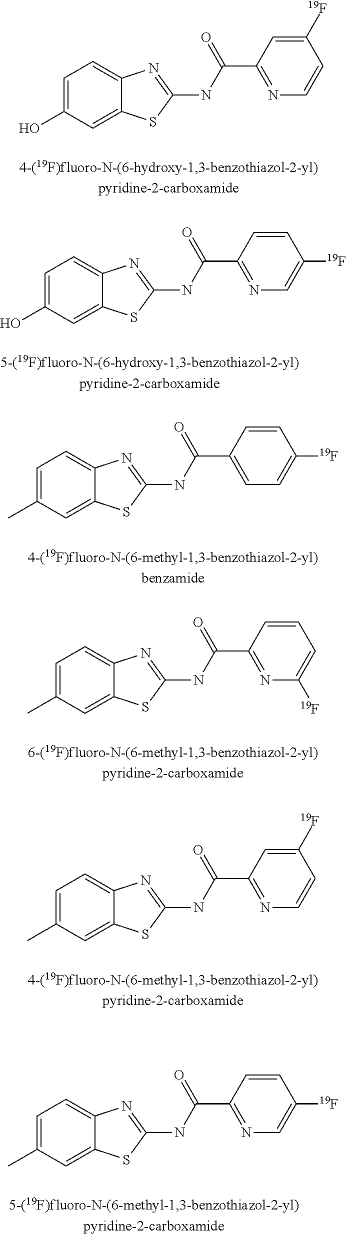

In a seventh aspect the present invention is directed to compounds of formula VI

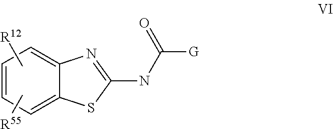

wherein
G is selected from the group comprising 1-(N—R¹¹)-2,3-dihydro-1H-indol-5-yl, 1-(N—R¹¹)-1H-indol-5-yl, phenyl and pyridyl, whereas G is substituted with R¹³ and R¹⁵.
R¹¹ is selected from the group comprising (C₁-C₄)alkyl, R¹⁸ and R¹⁴;
R¹² is selected from the group comprising hydrogen and R¹⁴—O—
R¹³ is selected from the group comprising hydrogen, (R¹⁴)O— and —N((C₁-C₄)alkyl)R¹⁴;
R¹⁴ is hydrogen:
R¹⁵ and R⁵⁵ are independently and individually selected from the group comprising hydrogen, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
R¹⁸ is a amine-protecting group;
including all isomeric forms of said compound, including but not limited to enantiomers and diastereoisomers as well as racemic mixtures,
and any pharmaceutically acceptable salt, ester, amide, complex or prodrug thereof;
with the proviso that compounds of formula IV contain exactly one R¹⁴.

In one embodiment G is selected from the group comprising 1-(N—R¹¹)-2,3-dihydro-1H-indol-5-yl and, 1-(N—R¹¹)-1H-indol-5-yl, whereas G is substituted with R¹⁵ and R¹²;

in one embodiment G is selected from the group comprising phenyl and pyridyl, whereas G is substituted with R¹⁵ and R¹³;
in a preferred embodiment G is selected from the group comprising phenyl and pyrid-2-yl, whereas G is substituted with R¹⁵ and R¹³;
in a preferred embodiment R¹¹ is selected from the group comprising methyl, R¹⁸ and R¹⁴;
in a preferred embodiment R¹³ is selected from the group comprising hydrogen, (R¹⁴)O— and —N(methyl)(R¹⁴);
in a preferred embodiment R¹⁵ and R⁵⁵ are independently and individually selected from the group comprising hydrogen, chloro, fluoro, methyl and methoxy;
in a preferred embodiment R¹⁸ is selected from the group comprising (tert-butoxy)-carbonyl, triphenylmethyl, ((para-methoxy)phenyl-diphenyl)methyl, (1-adamantyloxy)carbonyl, (diphenylmethoxy)carbonyl, (cinnamoyloxy)carbonyl, (cyclobutyloxy)carbonyl, ((1-methyl)cyclobutyloxy)carbonyl, ((1-methyl-1-phenyl)ethyloxy)carbonyl, ((1-methyl-1-(4-biphenylyl))ethyloxy)carbonyl, (vinyloxy)carbonyl, formyl, pivaloyloxymethyl and diphenylphosphinyl;
in a more preferred embodiment R¹⁸ is selected from the group comprising (tert-butoxy)-carbonyl, triphenylmethyl, (diphenylmethoxy)carbonyl, ((1-methyl-1-phenyl)ethoxy)carbonyl and formyl;
in an even more preferred embodiment R¹⁸ is selected from the group comprising (tert-butoxy)-carbonyl and formyl:
with the proviso that compounds of formula VI contain exactly one R¹⁴.

In an eighth aspect of the present invention is directed to a method for obtaining compounds of Formula I, wherein L is [¹⁸F]fluoro or [¹⁹F]fluoro.

Surprisingly three methods have been identified for obtaining such compounds.

In a first embodiment, a precursor compound according to formula I, wherein L is R³ as defined above, R⁷ is R¹⁷ as defined above and R⁸ is R¹⁸ as defined above is reacted with an F-fluorinating agent and optionally and subsequently the compound of formula I (e.g. wherein L=fluoro and wherein R⁷ or R⁸ is not hydrogen) is deprotected.

Preferably, said F-fluorinating agent is a compound comprising F-anions, preferably a compound selected from the group comprising 4,7,13,16,21,24-hexaoxa-1,10-diazabicyclo[8.8.8]-hexacosane KF, i.e. crownether salt Kryptofix KF, KF, HF, KHF₂, CsF, NaF and tetraalkylammonium salts of F, such as tetrabutylammonium fluoride, and wherein F=¹⁸F or ¹⁹F.

More specifically, with respect to ¹⁸F-labelled compounds having formula I, the first embodiment of a radiolabeling method for obtaining an ¹⁸F-labelled compound of formula I comprises the steps of
¹⁸F-Radiolabelling a compound of formula I having an appropriate leaving group with a fluorination agent for obtaining an ¹⁸F-labelled compound of formula I,
optionally deprotecting an amine- or phenol-protecting group within compounds of formula I wherein R⁷ or R⁸ are not hydrogen with a suited reagent for obtaining an ¹⁸F-labelled compound of formula I wherein R⁷ and R⁸ are hydrogen.

The term “suited reagent” as employed herein refers to reagents causing reaction conditions which are known or obvious to someone skilled in the art and which are chosen from but not limited to: acidic, basic, hydrogenolytical, oxidative, photolytical, preferably acidic cleavage conditions and which are chosen from but not limited to those described in Greene and Wuts, Protecting groups in Organic Synthesis, third edition, page 494-653 and 249-290, respectively.

The term “radiolabelling” a molecule, as used herein, usually refers to the introduction of an ¹⁸F-atom into the molecule.

The fluorination agent is defined as above, wherein F=¹⁸F.

In a preferred embodiment

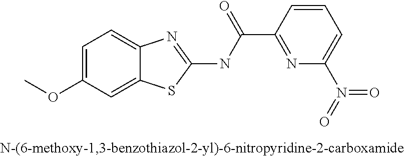

is reacted with a [¹⁸F]fluorination agent towards

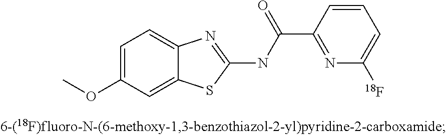

in another preferred embodiment

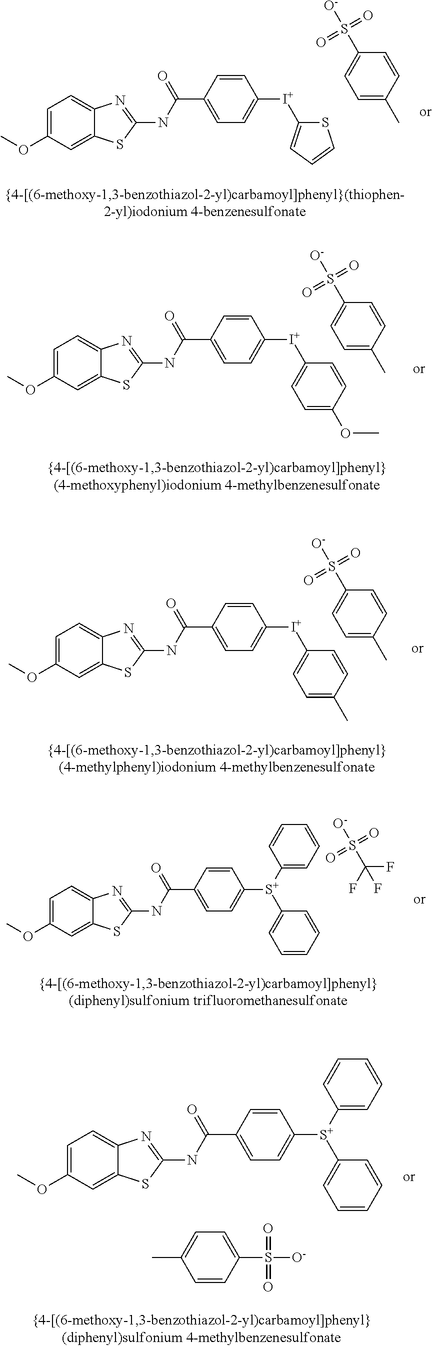

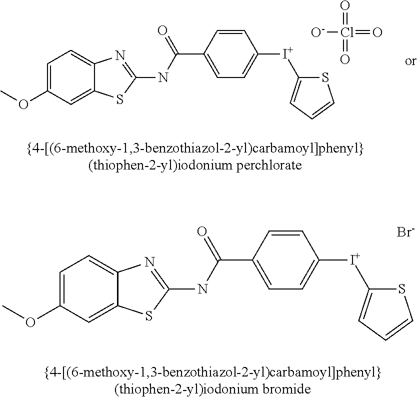

is reacted with a [¹⁸F]fluorination agent towards

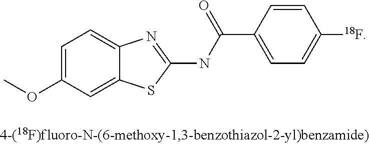

In a second embodiment, a method of synthesis of compounds of Formula Ib,

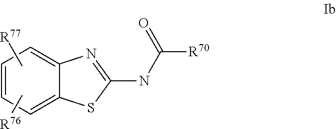

comprises the steps:
F-fluorinating a compound of formula V

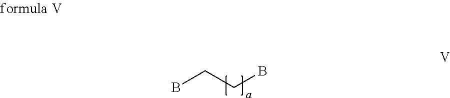

with an F-fluorinating agent to yield a compound of formula IV,

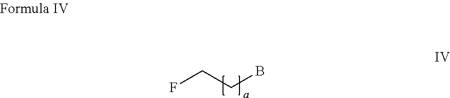

substituting said compound of formula IV with a compound of formula VI

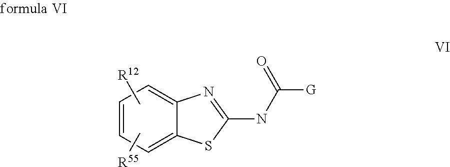

deprotection in those cases where compounds of formula VI comprise R¹⁸ or R¹⁷:
   wherein
   R⁷⁰ is selected from the group comprising 1-(N—R⁷¹)-2,3-dihydro-1H-indol-5-yl, 1-(N—R⁷¹)-1H-indol-5-yl, phenyl and pyridyl, whereas R⁷⁰ is substituted with R⁷³ and R⁷⁵;
   R⁷¹ is selected from the group comprising (C₁-C₄)alkyl, hydrogen, R¹⁸ and (L-CH₂—(CH₂)ₐ)—;
   R⁷³ is selected from the group comprising hydrogen, (L-CH₂—(CH₂)ₐ—)O—, —N(L-CH₂—(CH₂)ₐ—)(H) and —N((C₁-C₄)alkyl)(L-CH₂—(CH₂)ₐ—);
   R⁷⁵ and R⁷⁶ are independently and individually selected from the group comprising hydrogen, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
   R⁷⁷ is selected from the group comprising hydrogen and (L-CH₂—(CH₂)ₐ)—O; wherein L in Formula Ib is [¹⁸F]fluoro or [¹⁹F]fluoro, with the proviso that compounds of Formula Ib comprise exactly one L;
   F in Formula IV is [¹⁸F]fluoro or [¹⁹F]fluoro;
   a is an integer from 0 to 5, preferably from 0 to 2, more preferably from 0 to 1;
   B is a leaving group, preferably halo, in particular chloro, bromo, iodo, mesyloxy, tosyloxy, trifluormethylsulfonyloxy, nona-fluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutyl-phenyl)sulfonyloxy, and (4-methoxy-phenyl)sulfonyloxy;
   G is selected from the group comprising 1-(N—R¹¹)-2,3-dihydro-1H-indol-5-yl, 1-(N—R¹¹)-1H-indol-5-yl, phenyl and pyridyl, whereas G is substituted with R¹³ and R¹⁵.
   R¹¹ is selected from the group comprising (C₁-C₄)alkyl, R¹⁸ and R¹⁴;
   R¹² is selected from the group comprising hydrogen and (R¹⁴)O—;
   R¹³ is selected from the group comprising hydrogen, (R¹⁴)O—, —N(R¹⁴)(R¹⁸) and —N((C₁-C₄)alkyl)(R¹⁴);
   R¹⁴ is hydrogen:
   R¹⁵ and R⁵⁵ are independently and individually selected from the group comprising hydrogen, (R¹⁷)O—, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
   R¹⁷ is as defined above;
   R¹⁸ is as defined above;
   including all isomeric forms of said compound, including but not limited to enantiomers and diastereoisomers as well as racemic mixtures,
   and any pharmaceutically acceptable salt, ester, amide, complex or prodrug thereof;
   wherein said F-fluorinating agent is as defined above,
   and wherein F=¹⁸F or ¹⁹F,
   with the proviso that compounds of formula VI contain exactly one R¹⁴.

In a preferred embodiment B is selected from the group comprising iodo, bromo, chloro, mesyloxy, tosyloxy, trifluormethylsulfonyloxy, and nona-fluorobutylsulfonyloxy.

More specifically the second embodiment of a radiolabeling method for obtaining an ¹⁸F-labelled compound of formula I comprises the steps of
¹⁸F radiolabeling a compound of formula V with a [¹⁸F]fluorination agent to yield a compound of formula IV, and
substituting a compound of formula IV with a compound of Formula IV.

The ¹⁸F-labelled compound of Formula IV is

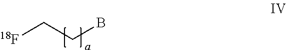

or pharmaceutically acceptable salts of an inorganic or organic acid thereof, hydrates, complexes, esters, amides, solvates or prodrugs thereof,
wherein
B is a leaving group;
the leaving group B is known or obvious to someone skilled in the art and which is taken from but not limited to those described or named in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O-nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15;
   in a more preferred embodiment B is selected from the group comprising:
a) ioda,
b) bromo,
c) chloro,
d) mesyloxy,
e) tosyloxy,
f) trifluormethylsulfonyloxy and
g) nonafluorobutylsulfonyloxy;
   a is an integer from 0 to 4, preferably a is an integer of from 0 to 2 and more preferably a is an integer of from 0 to 1;

The compound of Formula V is

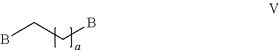

or pharmaceutically acceptable salts of an inorganic or organic acid thereof, hydrates, complexes, esters, amides, solvates or prodrugs thereof,
wherein
B is defined as above for compounds of Formula IV, and
   a is defined as above for compounds of Formula IV,
the fluorination agent is defined as above.

In a third embodiment, a method of synthesis of compounds of Formula Ic,

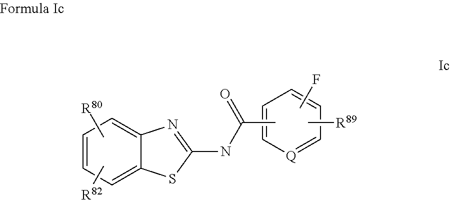

wherein F in Formula Ic is [¹⁸F]fluoro or [¹⁹F]fluoro, comprises the steps:
F-fluorinating a compound of formula XV

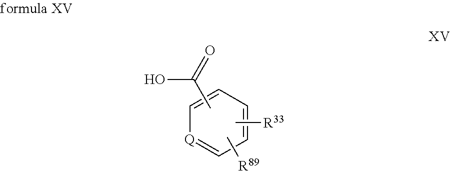

with an F-fluorinating agent to yield a compound of formula XIV,

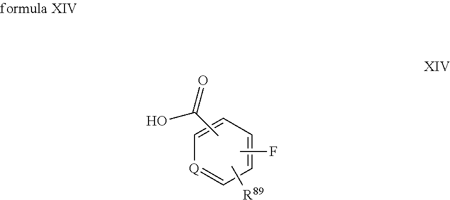

coupling said compound of formula XIV (or an activated derivative (e.g. active ester) of said compound of Formula XIV) with a compound of formula XVI

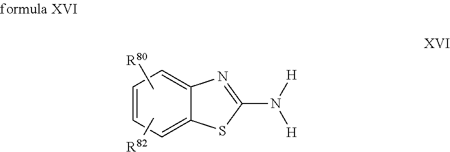

wherein F in Formula XIV and in Formula Ic is [¹⁸F]fluoro or [¹⁹F]fluoro;
in one embodiment F in Formula XIV and Ic is [¹⁸F]fluoro;
in one embodiment F in Formula XIV and Ic is [¹⁹F]fluoro;
Q is selected from the group comprising nitrogen and C(H);
to R³³ is as defined as above;
R⁸⁹ is selected from the group comprising hydrogen, (C₁-C₅)alkyl, (C₂-C₅)alkenyl, (C₁-C₅)alkoxy, halo, trifluoromethyl, cyano, —C(O)O—((C₁-C₅)alkyl), —N(R¹⁸)((C₁-C₄)alkyl) and —N((C₁-C₄)alkyl)₂;
R¹⁸ is as defined above;
R⁸⁰ and R⁸² are independently and individually, at each occurrence, selected from the group comprising hydrogen, halo, cyano, trifluoromethyl, (C₁-C₅)alkyl, (C₂-C₅)alkynyl, (C₂-C₅)alkenyl, (C₁-C₅)alkoxy and (R¹⁷)O—;
R¹⁷ is as defined above;
in one embodiment F is [¹⁸F]fluoro;
in another embodiment F is [¹⁹F]fluoro;
in a preferred embodiment Q is C(H);
R²⁵ is as defined above;
R²⁶ is as defined above;
in a preferred embodiment R⁸⁹ is selected from the group comprising hydrogen, (C₁-C₄)alkyl, halo, trifluoromethyl, cyano, —N(R¹⁸)((C₁-C₂)alkyl) and —N((C₁-C₂)alkyl)₂;
in a more preferred embodiment R⁸⁹ is selected from the group comprising hydrogen, methyl, bromo, fluoro, trifluoromethyl, cyano, —N(R¹⁸)(methyl) and —N(methyl)₂;
in an even more preferred embodiment R⁸⁹ is selected from the group comprising hydrogen, methyl, bromo, —N(R¹⁸)(methyl) and —N(methyl)₂;
in the most preferred embodiment R⁸⁹ is hydrogen;
in a preferred embodiment R⁸⁰ and R⁸² are independently and individually, at each occurrence, selected from the group comprising hydrogen, halo, (C₁-C₄)alkyl, (C₁-C₄)alkoxy, (R¹⁷)O—;
in a more preferred embodiment R⁸⁰ and R⁸² are independently and individually, at each occurrence, selected from the group comprising hydrogen, fluoro, iodo, (C₁-C₃)alkyl and (C₁-C₃)alkoxy;
in an even more preferred embodiment R⁸⁰ and R⁸² are independently and individually, at each occurrence, selected from the group comprising hydrogen, methyl, ethyl and methoxy;
in a preferred embodiment

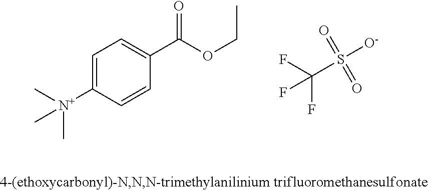

is reacted with a [¹⁸F]fluorination agent and converted subsequently to the active ester (compare Kabalka et al., Journal of Labeled Compounds and Radiopharmaceuticals, (2008), 51, 68-71) obtaining

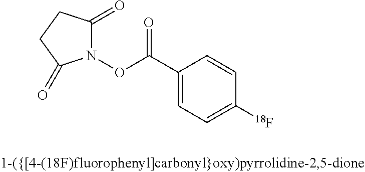

which is subsequently reacted with

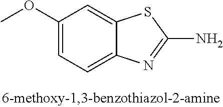

towards

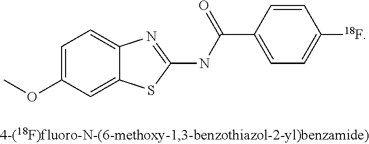

In a preferred embodiment, the fluorination agent is a fluorine radioactive isotope derivative.

More preferably the fluorine radioactive isotope derivative is a ¹⁸F derivative. More preferably, the ¹⁸F derivative is 4,7,18,16,21,24-Hexaoxa-1,10-diazabicyclo[8.8.8]-hexacosane K¹⁸F (crownether salt Kryptofix K¹⁸F), K¹⁸F, H¹⁸F, KH¹⁸F₂, Cs¹⁸F, Na¹⁸F or tetraalkylammonium salt of ¹⁸F (e.g. [F-18]tetrabutylammonium fluoride). More preferably, the fluorination agent is K¹⁸F, H¹⁸F, or KH¹⁸F₂, most preferably K¹⁸F (¹⁸F fluoride anion).

The radiofluorination reaction can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in dimethylformamide with potassium carbonate as base and “kryptofix” as crown-ether. But also other solvents can be used which are well known to experts. These possible conditions include, but are not limited to: dimethylsulfoxid and acetonitril as solvent and tetraalkyl ammonium and tertraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). The radiofluorination can be carried out in a “hot-cell” and/or by use of a module (eview: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated or semi-automated synthesis.

The term “coupling said compound of formula XIV with a compound of formula XVI” includes the case that a compound of formula XIV is activated in a way which is known to someone skilled in the art. Thus, the carboxylic acid being part of compound XIV and XV can be reacted with an activating reagent which is known to someone skilled in the art and which is chosen from but not limited to: N-succinimide, diisopropylcarbodiimide dicyclohexylcarbodiimide, HOBT, TFFH, PyBOP, HATU, PyAOP, (see e.g. Chan and White (“Fmoc Solid Phase Peptide Synthesis—A Practical Approach”, 2000, chapter 7,) but also other condensating agents (e.g. TBCR (J. Am. Chem. Soc. 2005, 127, 16912-16920)) resulting in an activated species (also called “active ester” by someone skilled in the art) which is either isolated and subsequently coupled or which is coupled in situ with compounds of formula XVI.

A ninth aspect of the present invention is directed to a composition comprising a compound according to the present invention and a pharmaceutically acceptable carrier or diluent.

In one embodiment said compound is an ¹⁸F-labelled compound.

In another embodiment said compound is a ¹⁹F-labelled compound.

In yet another embodiment said compound is a precursor compound.

The invention also provides for a compound according to the present invention, preferably an ¹⁸F- or ¹⁹F-labelled compound according the present invention, or a composition according to the present invention for use as a pharmaceutical or diagnostic agent or imaging agent.

The invention also provides for the use of a compound according to the present invention, preferably an ¹⁸F- or ¹⁹F-labelled compound according to the present invention, or a composition according to the present invention for the manufacture of a medicament for the treatment and/or diagnosis and/or imaging of diseases of the central nervous system (CNS).

The invention also provides for an ¹⁸F-labelled compound of formula I or a composition containing such compound for use as a diagnostic agent or imaging agent, in particular for diseases of the central nervous system.

A tenth aspect of the present invention is directed to a kit comprising a sealed vial containing a predetermined quantity of a compound

a) which is a precursor compound having formula
b) a compound of formula V and a compound of formula VI, as defined above or
c) a compound of formula XV and a compound of formula XVI as defined above.

The invention also provides for a method for detecting the presence of A-beta amyloid plaques in a patient's body, preferably for imaging a disease of the central nervous system in a patient, comprising:

introducing into a patient's body a detectable amount of an ¹⁸F-labelled compound according to the present invention or a composition comprising such compound,
and detecting said compound or said composition by positron emission tomography (PET).

The invention also provides for a method of treatment of a disease of the central nervous system comprising the step of introducing into a patient a suitable quantity of a compound according to the present invention, preferably of an ¹⁸F- or ¹⁹F-labelled compound according to the present invention.

An eleventh aspect of the present invention is directed to a method for obtaining precursor compounds having formula I wherein L is R³ as defined above, R⁷ is R¹⁷ as defined above and R⁸ is R¹⁸ as defined above.

Surprisingly two methods have been identified for obtaining such compounds.

In one embodiment the present invention comprises a method for obtaining precursor compounds having formula I wherein L is R³ as defined above, R³ is R³⁴ as defined above, R⁷ is R¹⁷ as defined above and R⁸ is R¹⁸ as defined above

comprises the step:
reacting a starting compound of Formula I
   wherein L is R¹¹) as defined above, R⁷ is R¹⁷ as defined above, R⁸ is R¹⁸ as defined above and R¹⁰ is R³⁰ as defined above with an “electrophilization reagent”.

In a preferred embodiment the present invention comprises a method for obtaining precursor compounds having formula I wherein L is R³ as defined above, R³ is R³⁴ as defined above, R⁷ is R¹⁷ as defined above and R⁸ is R¹⁸ as defined above; wherein L and R³ are attached to a sp³-hybridized carbon atom, comprises the step:
reacting a starting compound of Formula I
   wherein L is R¹⁰ as defined above, R⁷ is R¹⁷ as defined above, R⁸ is R¹⁸ as defined above and R¹⁰ is R³⁰ as defined above
   with an “electrophilization reagent”;
   with the proviso that wherein L, R¹⁰ and R³ are attached to a sp³-hybridized carbon atom,

In another embodiment the present invention comprises a method for obtaining precursor compounds having formula I wherein L is R³ as defined above, R³ is R³³ as defined above, R⁷ is R¹⁷ as defined above and R⁸ is R¹⁸ as defined above

comprises the step:
reacting a starting compound of Formula I
   wherein L is R¹⁰ as defined above, R⁷ is R¹⁷ as defined above, R⁸ is R¹⁸ as defined above and R¹⁰ is R²⁰ as defined above
   with an aromatic hypervalent iodo-compound or an oxidizing agent or methylation reagent;

In a preferred embodiment the present invention comprises a method for obtaining precursor compounds having formula I wherein L is R³ as defined above, R³ is R³³ as defined above, R⁷ is R¹⁷ as defined above and R⁸ is R¹⁸ as defined above

comprises the step:
reacting a starting compound of Formula I
   wherein L is R¹⁰ as defined above, R⁷ is R¹⁷ as defined above, R⁸ is R¹⁸ as defined above and R¹⁰ is R²⁰ as defined above
   with an aromatic hypervalent iodo compound or an oxidizing agent or methylation reagent; with the proviso that L, R²⁰ and R³ which are included in compounds having formula I are attached to a sp² hybridized carbon atom.

In yet another embodiment present invention comprises a method for obtaining precursor compounds having formula I wherein L is R¹⁰ as defined above, R¹⁰ is R²⁰ as defined above, R⁷ is R¹⁷ as defined above and R⁸ is R¹⁸ as defined above

comprises the step:
reacting a compound of Formula XII

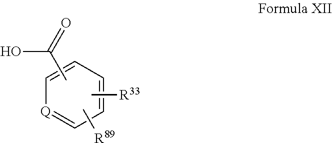

with a compound of Formula XVI

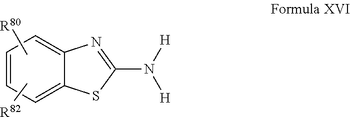

wherein
R³³, R⁸⁹, R⁸⁰ and R⁸² are as defined above.

The term “electrophilisation reagent” as employed herein by itself or as part of another group is known or obvious to someone skilled in the art, and is suited to convert a hydroxy group being attached to a sp³ hybridized carbon atom to a leaving group and which is chosen from but not limited to thionyl chloride (e.g. Organic and Biomolecular Chemistry; 4; 22; (2006); 4101-4112), phosphorus pentachloride (e.g. Bioorganic and Medicinal Chemistry; 16; 6; (2008); 3309-3320), methanesulfonyl chloride (e.g. Organic and Biomolecular Chemistry; English; 4; 24; (2006); 4514-4525), carbon tetrachloride/triphenylphosphine (Tetrahedron: Asymmetry; English; 19; 5; 2008; 577-583), hydrogen chloride (e.g. Russian Chemical Bulletin; English; 56; 6; 2007; 1119-1124), N-chloro-succinimide/dimethylsulfide (e.g. Bioscience, Biotechnology, and Biochemistry 72; 3; (2008); 851-855), hydrogen bromide (e.g. Journal of Labelled Compounds and Radiopharmaceuticals; 51; 1; (2008); 12-18), phosphorus tribromide (Journal of the American Chemical Society; 130; 12; (2008); 3726-3727), carbon tetrabromide/triphenylphosphine (e.g. Journal of the American Chemical Society; 130; 12; (2008); 4153-4157), N-bromo-succimide/SMe₂ (e.g. Chemical Communications (Cambridge, United Kingdom); 1; (2008); 120-122), bromine/triphenylphosphine (e.g. Journal of the American Chemical Society; 130; 12; (2008); 4153-4157), N-bromo-succimide/SMe₂ (e.g. Chemical Communications (Cambridge, United Kingdom); 1; (2008); 120-122), Br₂/PPh₃ (e.g. European Journal of Organic Chemistry; 9; (2007); 1510-1516), mesylchloride, tosylchloride, trifluormethylsulfonylchloride, nona-fluorobutylsulfonylchloride, (4-bromo-phenyl)sulfonylchloride, (4-nitro-phenyl)sulfonylchloride, (2-nitro-phenyl)sulfonylchloride, (4-isopropyl-phenyl)sulfonylchloride, (2,4,6-tri-isopropyl-phenyl)sulfonylchloride, (2,4,6-trimethyl-phenyl)sulfonylchloride, (4-tertbutyl-phenyl)sulfonylchloride, (4-methoxy-phenyl)sulfonylchloride, mesylanhydride, tosylanhydride, trifluormethylsulfonylanhydride, nona-fluorobutylsulfonylanhydride, (4-bromo-phenyl)sulfonylanhydride, (4-nitro-phenyl)sulfonylanhydride, (2-nitro-phenyl)sulfonylanhydride, (4-isopropyl-phenyl)sulfonylanhydride, (2,4,6-tri-isopropyl-phenyl)sulfonylanhydride, (2,4,6-trimethyl-phenyl)sulfonylanhydride, (4-tertbutyl-phenyl)sulfonylanhydride, (4-methoxy-phenyl)sulfonylanhydride, ect.

The term “hypervalent iodo-compound or an oxidizing agent” as employed herein by itself or as part of another group is known or obvious to someone skilled in the art, and is suited to convert a stannyl-, iodo or borane-group being attached to a sp² hybridized carbon atom, to a leaving group being part of precursor compounds having formula I wherein R³³ is —I⁺(R²⁶)(X⁻) or —I⁺(R²⁵)(X⁻) and which is chosen from but not

limited to iodosobenzene diacetate, Koser's reagent (J. Org. Chem. 1977, 42, 1476) ect. (compare e.g. Tetrahedron Letters 48 (2007) 8632-8635, J. Labelled Compd. Radiopharm. (2004), 47, 429; Synthesis, (1994), 147; e.g. J. Chem. Soc., Chem. Commun. (1995), 21, 2215, J. Labelled Compd. Radiopharm. (1997), 40, 50; J. Chem. Soc. Perkin Trans. 1 (1998), 2043; Chem. Commun., (2000), 649); boronic-group: e.g. Tetrahedron; 63; 46; (2007); 11349-11354)

The term “methylating agent” as employed herein by itself or as part of another group is known or obvious to someone skilled in the art, and is suited to convert a dimethyl amino group being attached to a sp² hybridized carbon atom of a starting compound having formula I whereas R²⁰ is NMe₂, to a leaving group being part of precursor compounds having formula I wherein R³³ is —N⁺Me₃(X⁻) and which is chosen from but not limited to methyl iodide (Journal of Organic Chemistry; 72; 14; (2007); 5046-5055) and methyl triflate (e.g. Journal of Medicinal Chemistry; 50; 23; (2007); 5752-5764)

It is obvious that to someone skilled in the art that precursor compounds having formula I can be possibly converted into each other; e.g. a compound wherein precursor compound having formula I comprises a sulfonate ester, e.g. a mesyloxy or tosyloxy group, can be converted e.g. to a corresponding chloride (e.g. New Journal of Chemistry; 32; 3; (2008); 547-553) or bromide (e.g. Journal of the American Chemical Society; 130; 9; (2008); 2722-2723),

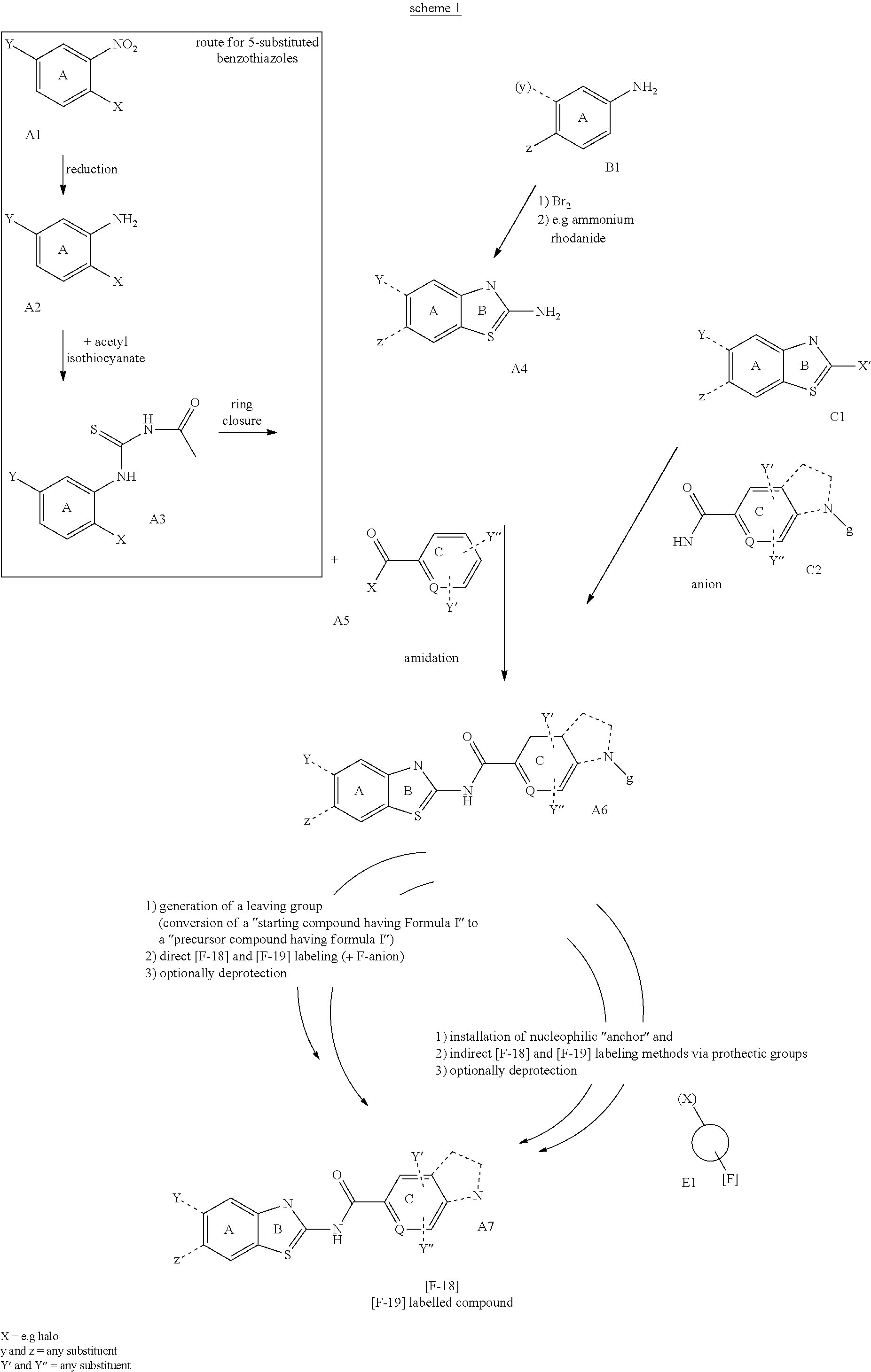

The general strategy for the synthesis of compounds of formula I comprising ring systems A, B and C is shown in scheme 1: Thus, compounds of type A4 are converted with carboxylic acid derivatives (A5) in an amidation reaction towards compounds of Formula I (e.g. A6). Those reactions are known to persons skilled in the art. A typical reaction is wherein A5 is a carboxylic acid chloride which is converted with a compound of type A4 to obtain a compound of type A6 (compare Heterocycles; 68; 11; (2006); 2285-2299). Compounds of type A4 can either be prepared via the route A1→A2→A3→A4 wherein nitro compounds of type A1 are reduced to aniline derivatives A2 which are converted with acetyl isothiocyanate towards compounds of type A3. These derivatives can undergo a ring closure reaction using base towards compounds of type A4 (e.g. Bioorganic and Medicinal Chemistry Letters; 15; 14; 2005; 3328-3332). Another approach to obtain compounds of type A4 is the halogenation (e.g. bromination) of pare substituted aniline derivatives which undergo subsequently ring closure reactions by use of rhodanide salts (e.g. ammonium rhodanide). Another approach could be the conversion of compounds of type A7 wherein X′ is a halo, preferably bromo or chloro, with the anion of an amide (compound of type A8). Those type of reactions are known in literature European Journal of Medicinal Chemistry, 13, (1978), 171-175.

Some particular examples are shown in scheme 2: e.g. compound 7 can be converted to compound 9 generating an amide bond using carboxylic acid 8 and condensating agent TBCR (J. Am. Chem. Soc. 2005, 127, 16912-16920) or carboxylic acid chloride 10. The corresponding precursor molecule 13 can be synthesized from carboxylic acid 11, which is converted to the intermediate sulfonium derivative 12 using diisopropyl magnesium bromide-THF solution, sodium hydride, 1,1′-dibenzene sulfinyl and trimethylsilyl trifluoromethanesulfonate (compare Synthesis (2002), 565-596 and Synthesis (2004), 1648-1654), and subsequent condensation with TBCR (J. Am. Chem. Soc., 2005, 127, 16912-16920). Also other amidation conditions are possible: which are chosen from but not limited to: succinimide, diisopropylcarbodiimide dicyclohexylcarbodiimide, HOBT, TFFH, PyBOP, HATU, PyAOP, (see e.g. Chan and White (“Fmoc Solid Phase Peptide Synthesis—A Practical Approach”, 2000, chapter 7.). Compound 13 can be converted into the F-18 labelled derivative 14 using a fluorination agent, e.g. [F-18]potassium fluoride and kryptofix in DMF. Another example for obtaining compounds of formula I is realized by the reaction of amine 7 with carboxylic acid 17 (ABCR) using TBCR as condensating agent. The aromatic nitro derivative 18 is fluorinated with [F-18]potassium fluoride and kryptofix towards [F-18] labelled compound 19. The corresponding F-19 derivative 16 is synthesized from amine 7 and carboxylic acid 15 by a amide-bond-formation reaction which are known to persons skilled in the art.

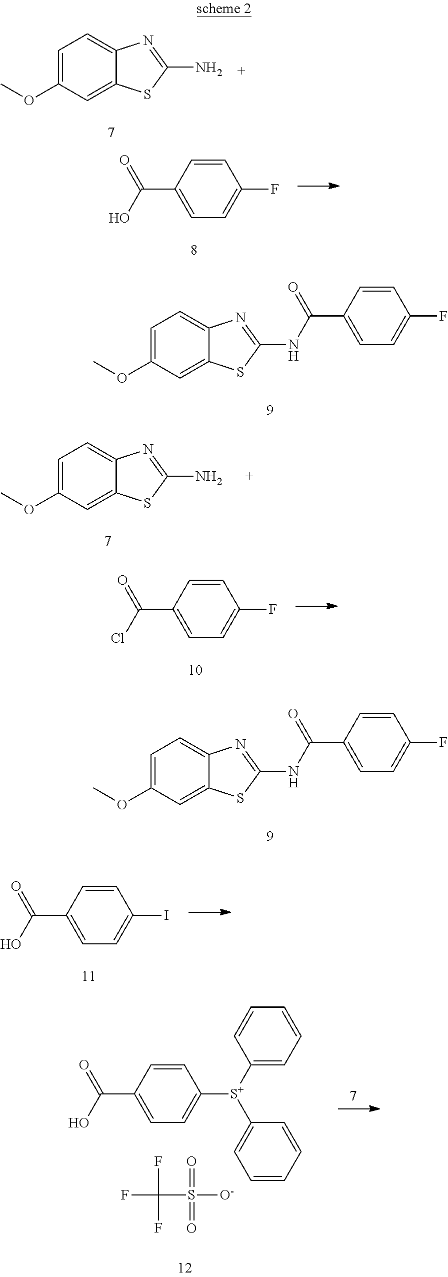

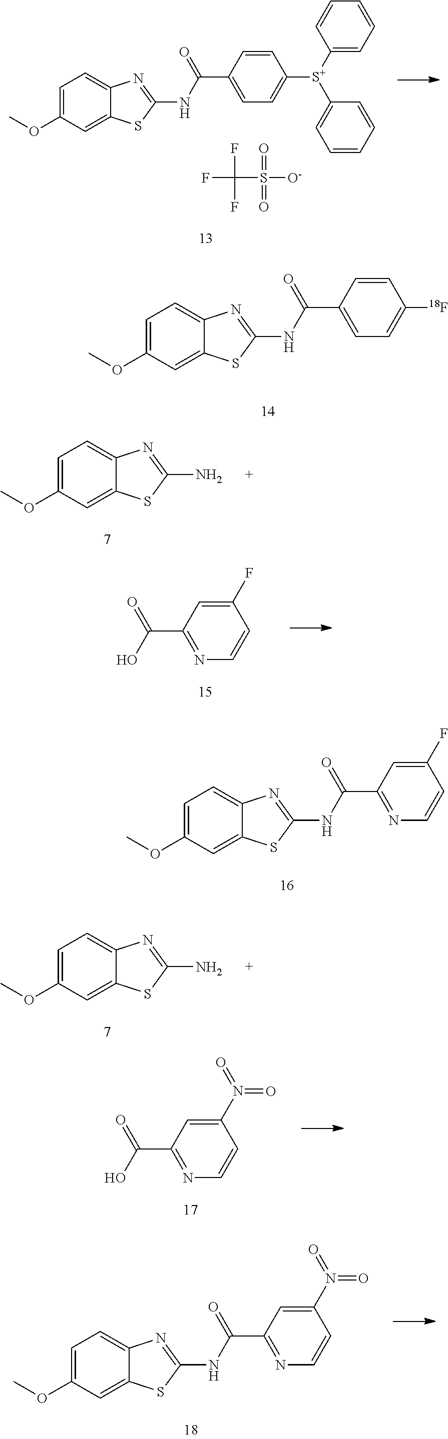

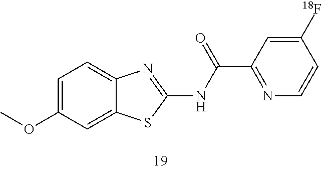

Compounds of formula I presented by compounds 22, 23, 25 and 26 can be prepared by corresponding procedures (scheme 3). Thus, amine 20 (Aldrich) is condensed with carboxylic acid 21 (Butt-Park) towards amide 22. The Boc protecting group is cleaved with a mixture of dichloromethane and trifluoro acetic acid to obtain standard reference compound 23. The ode derivative 25 is synthesized from amine 24 (Spectra) and carboxylic acid 21, whereas the precursor 26 is synthesized from 25 using thiophene and m-CPBA (Synlett (2008), No. 4, 592-596). The methods for radiofluorination towards F-18 labelled derivative 27 from iodonium derivative 26 including acidic deprotection of the Boc-protecting group are known to experts in the field.

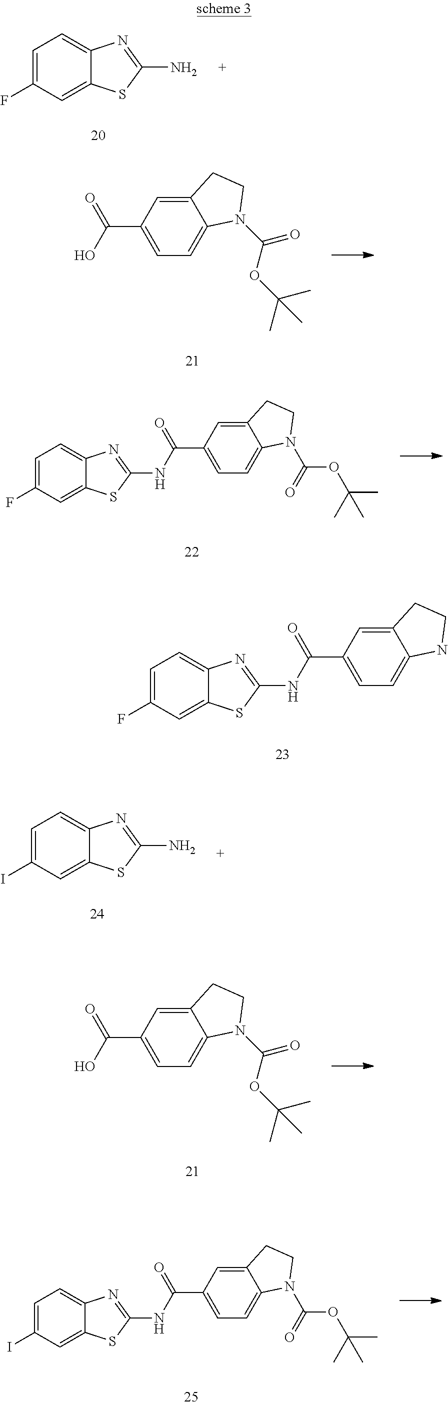

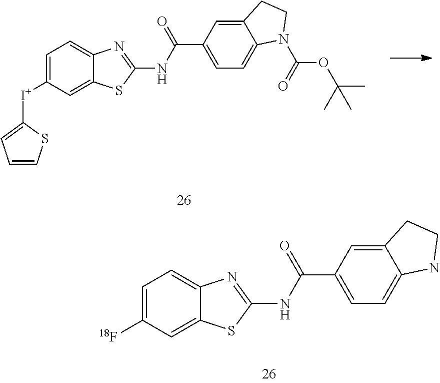

Similar precursor compounds having formula I which can be generated by described methods are:

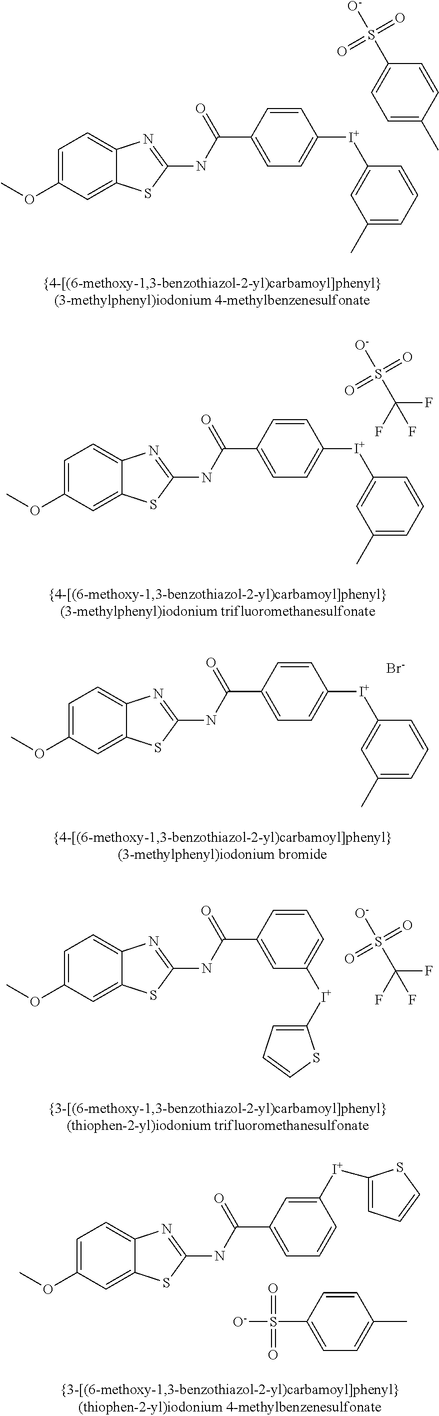

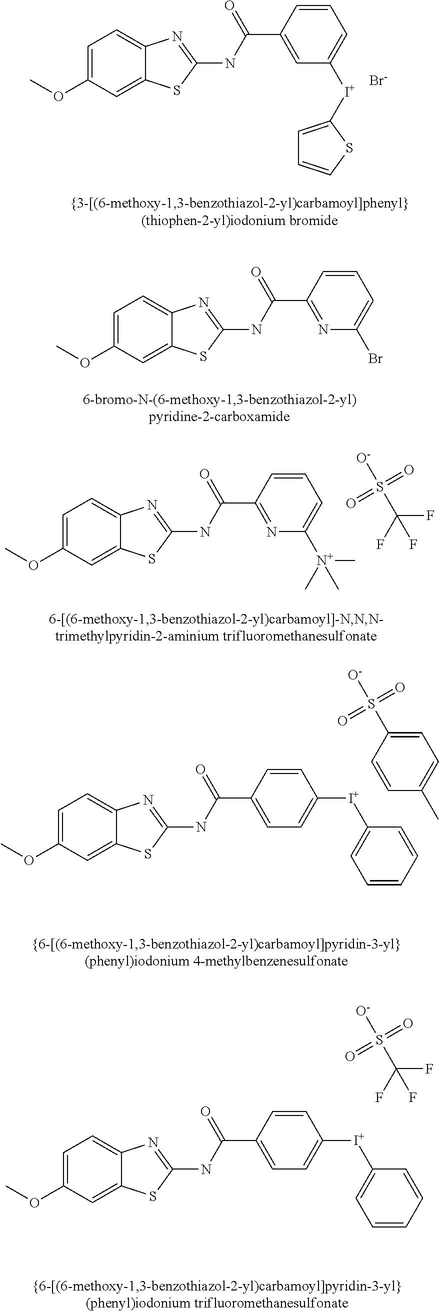

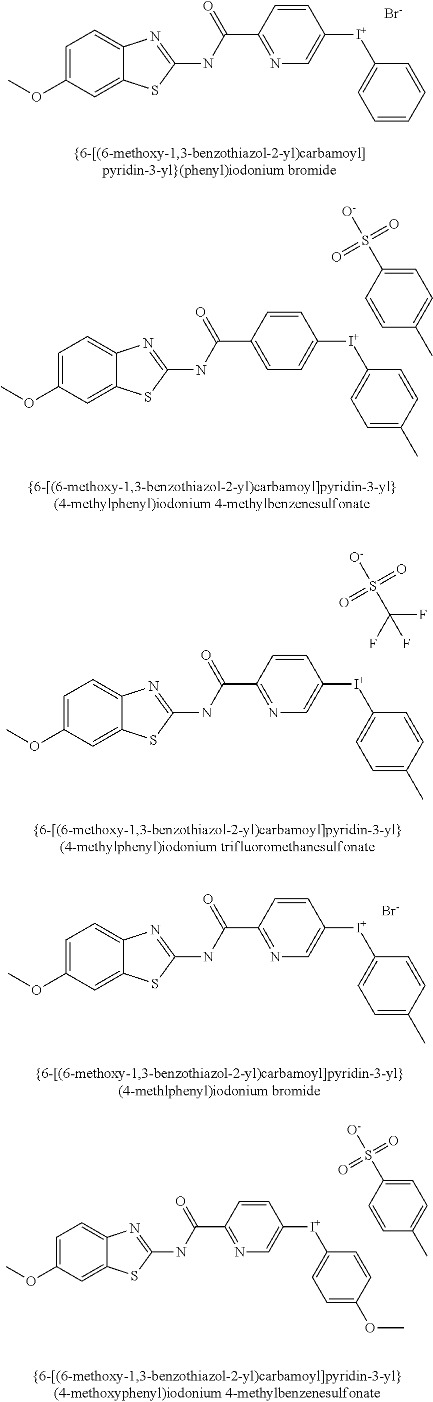

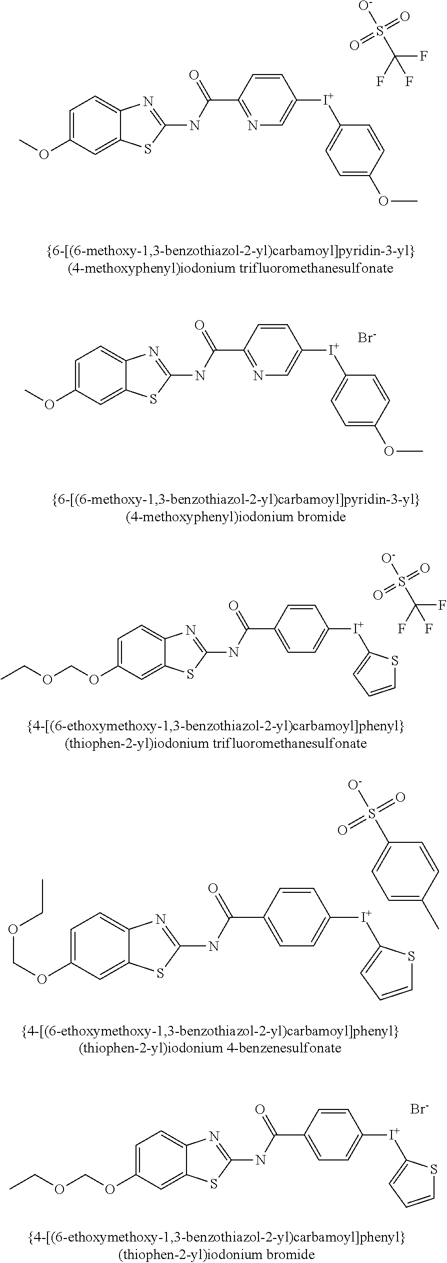

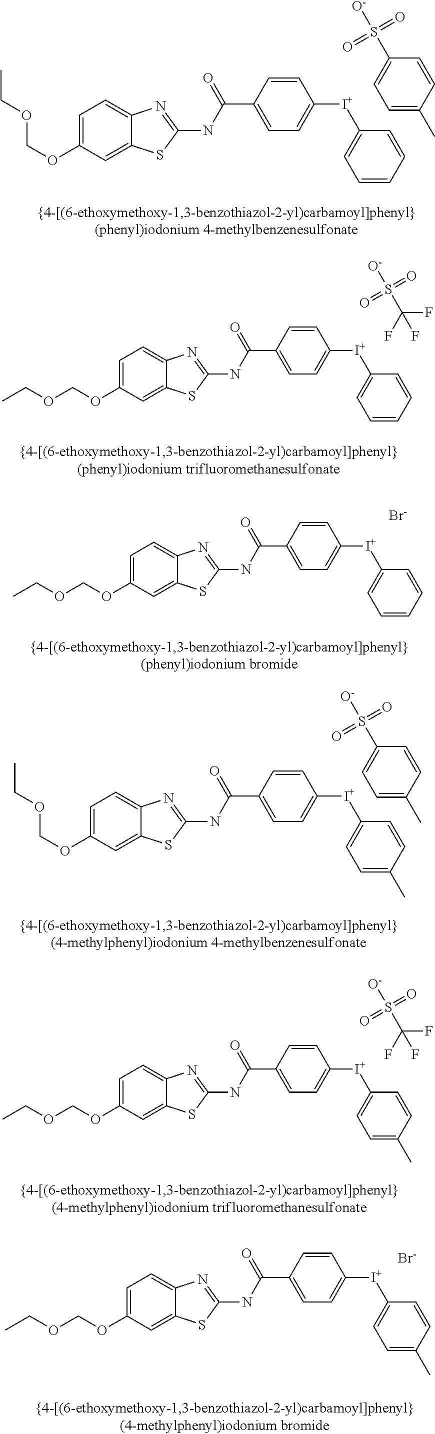

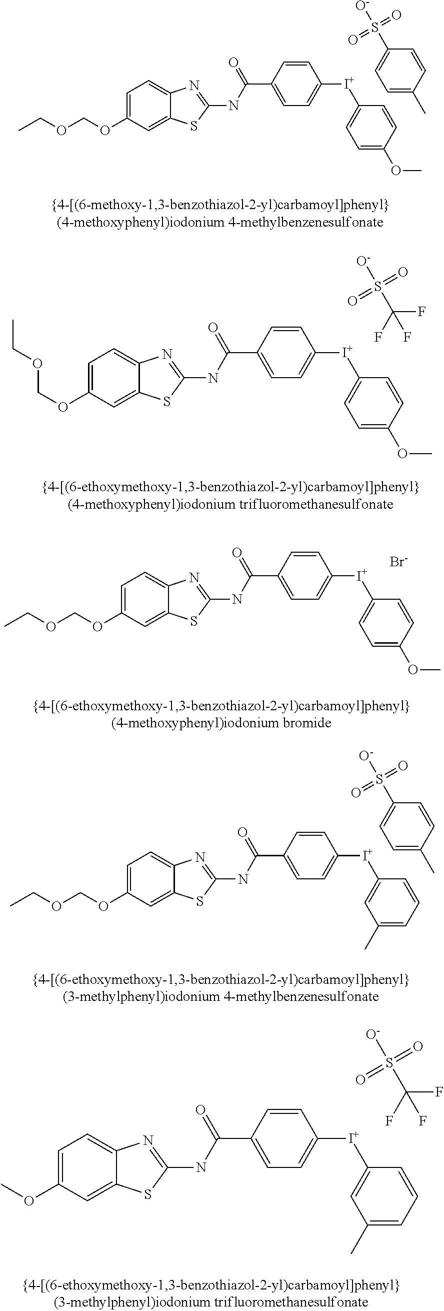

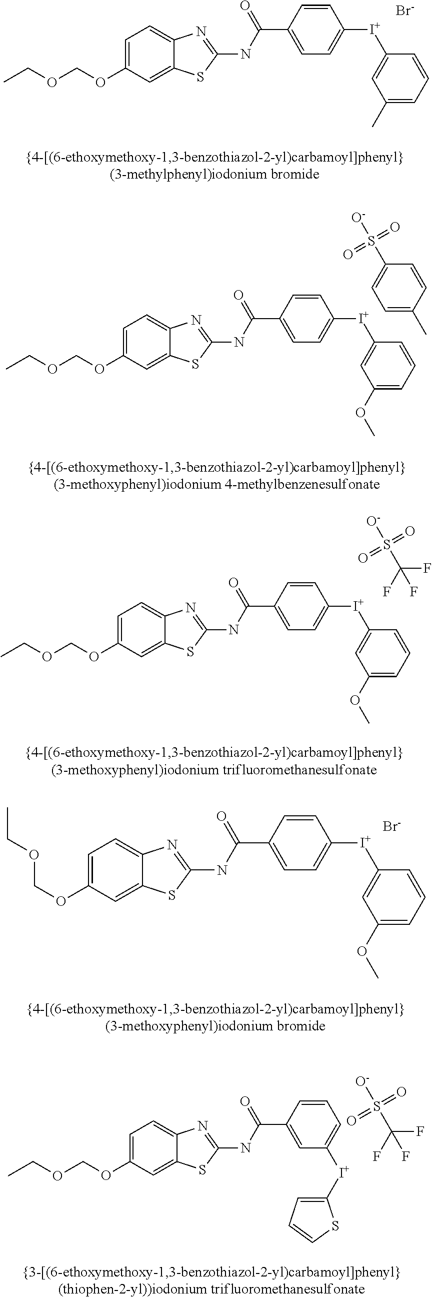

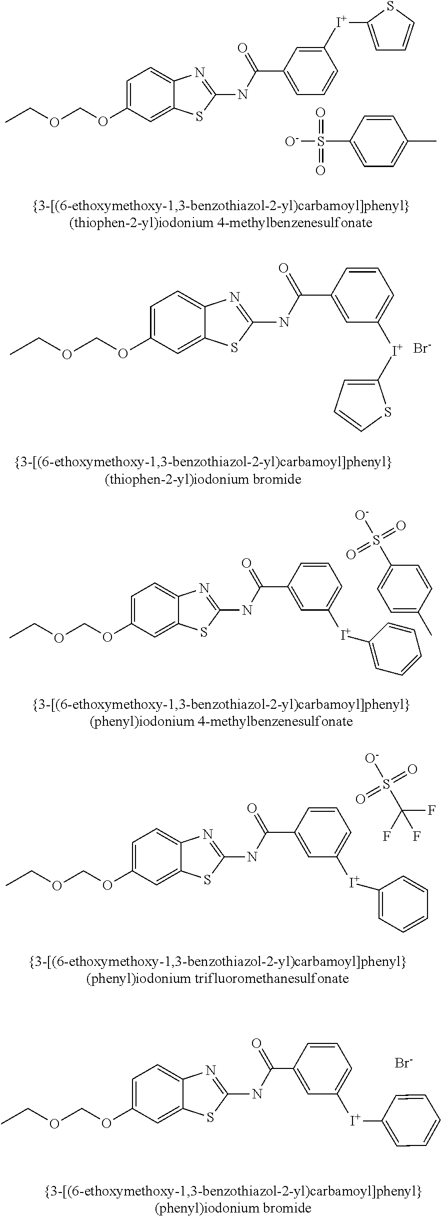

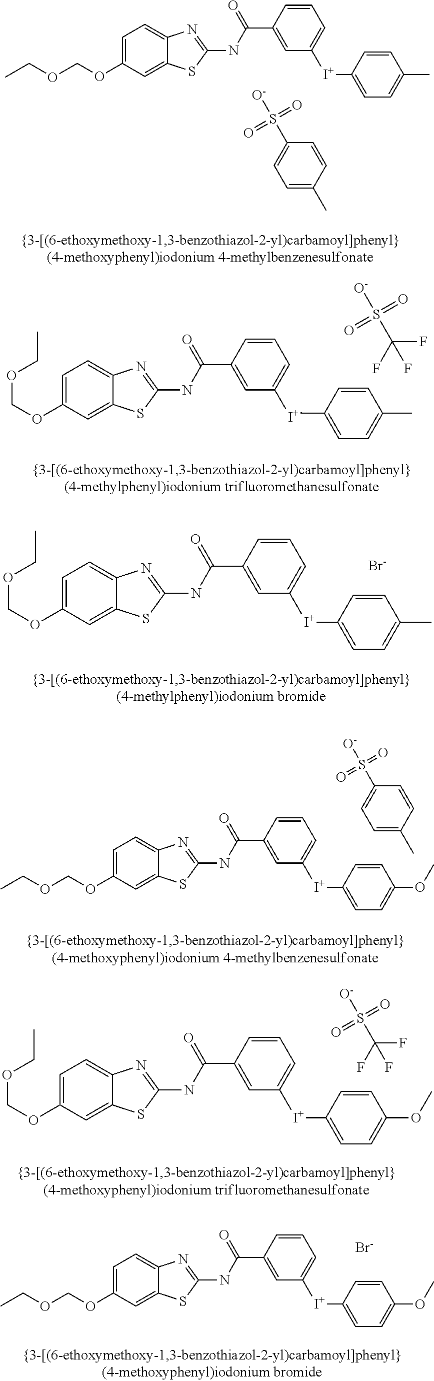

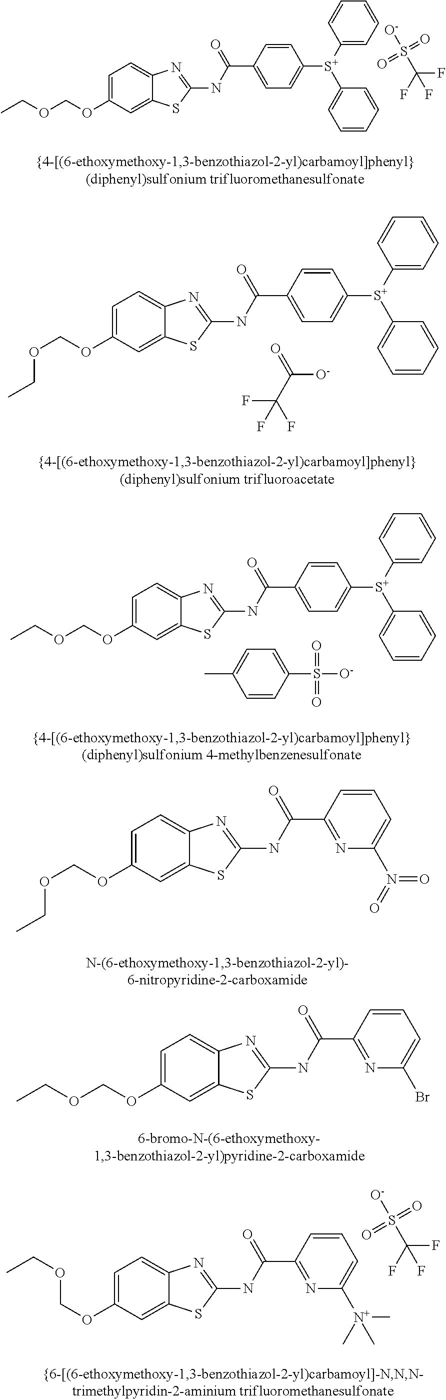

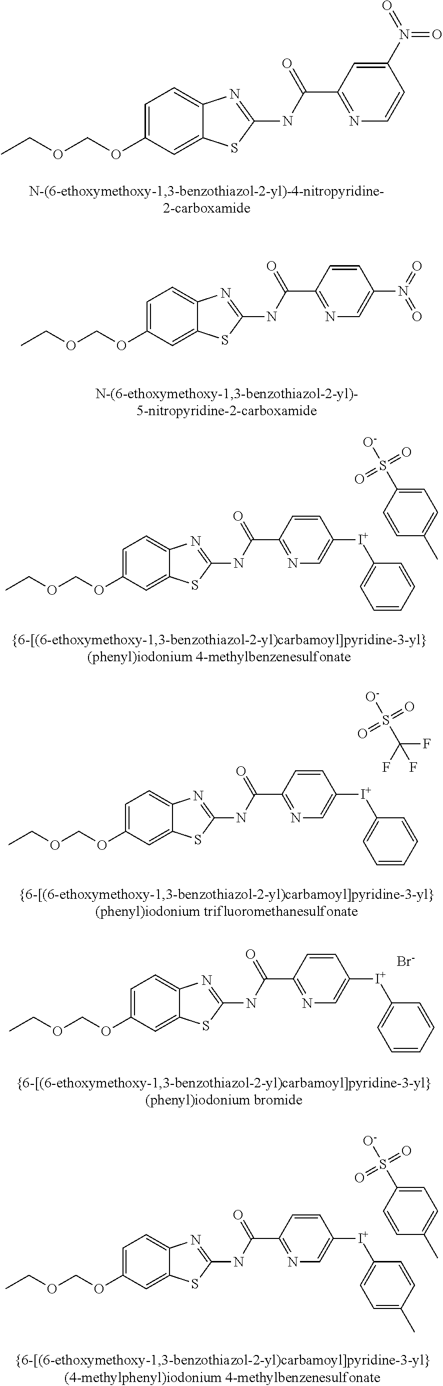

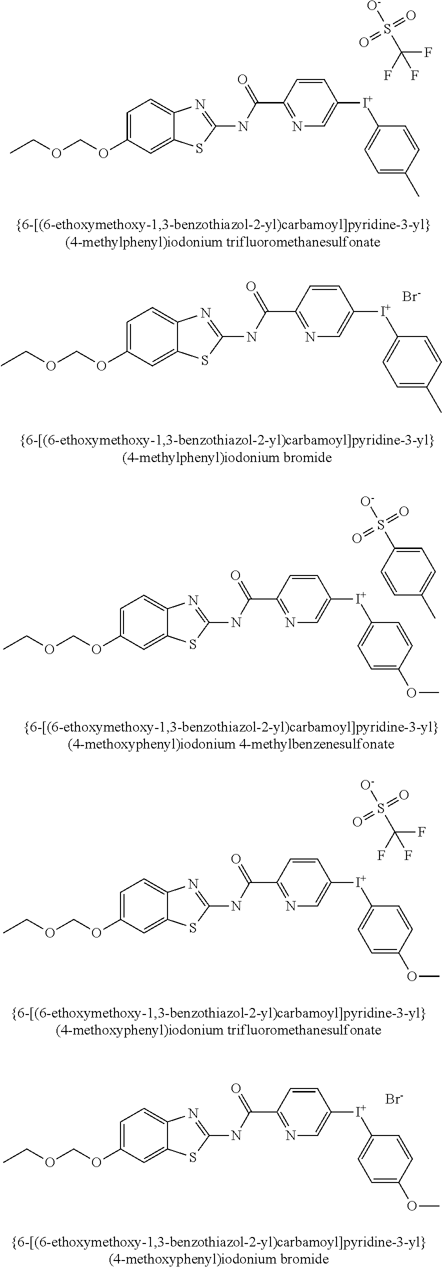

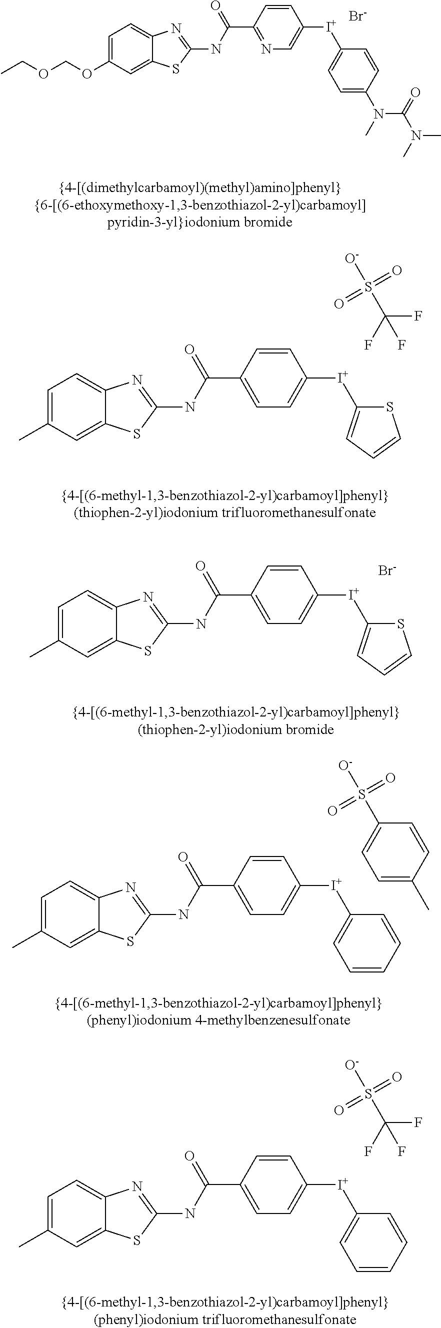

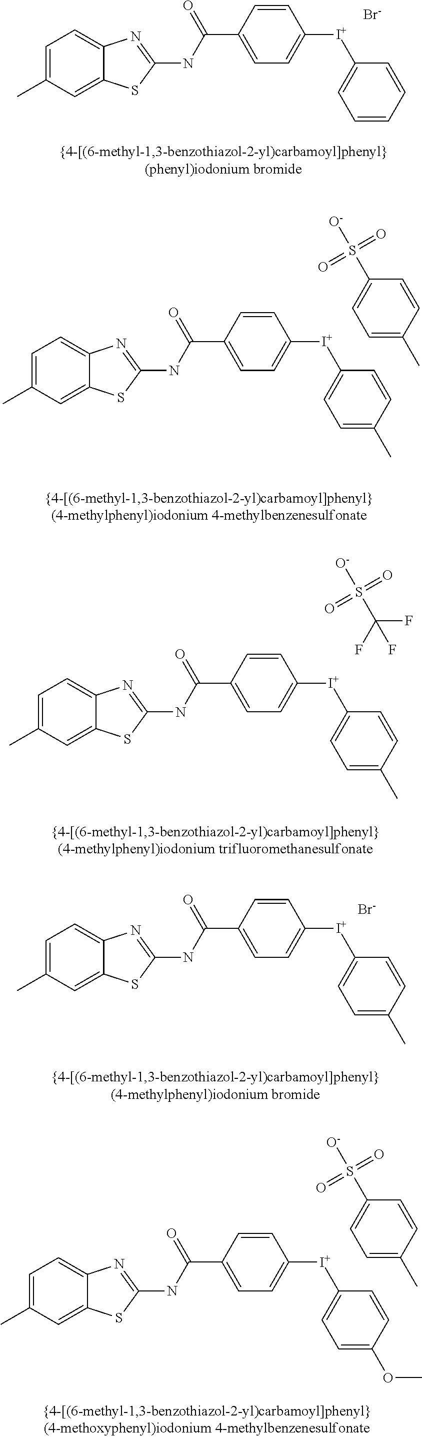

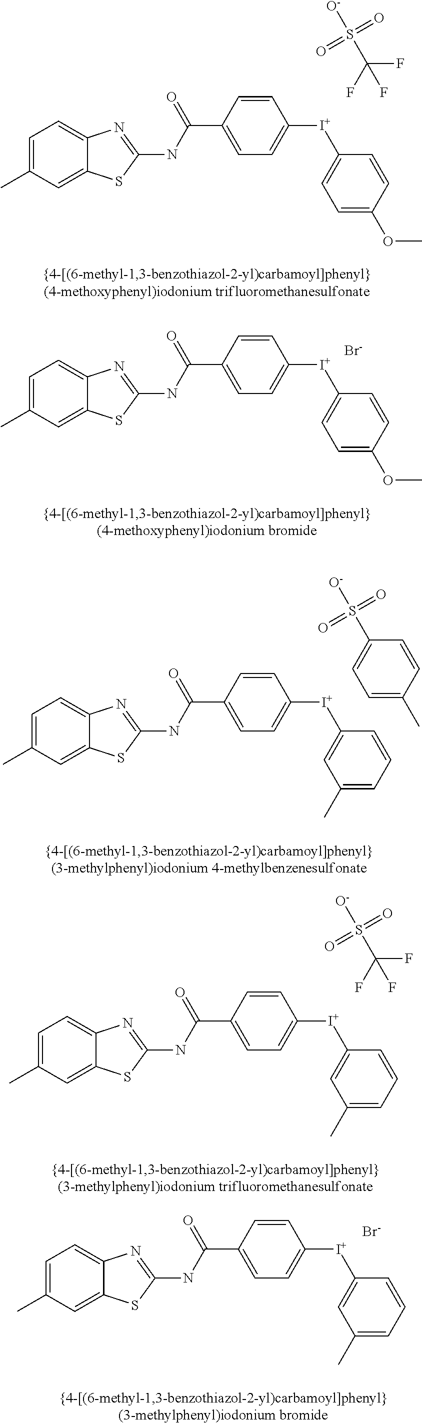

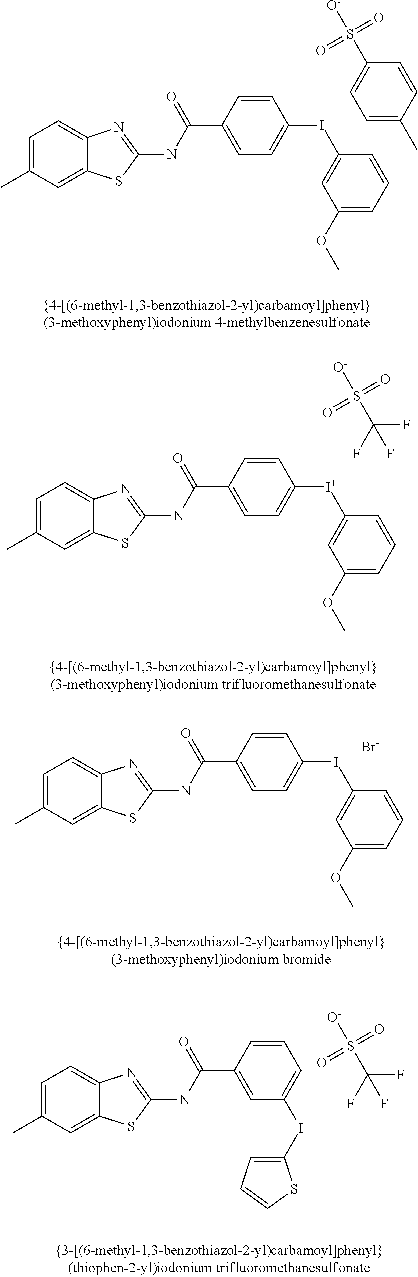

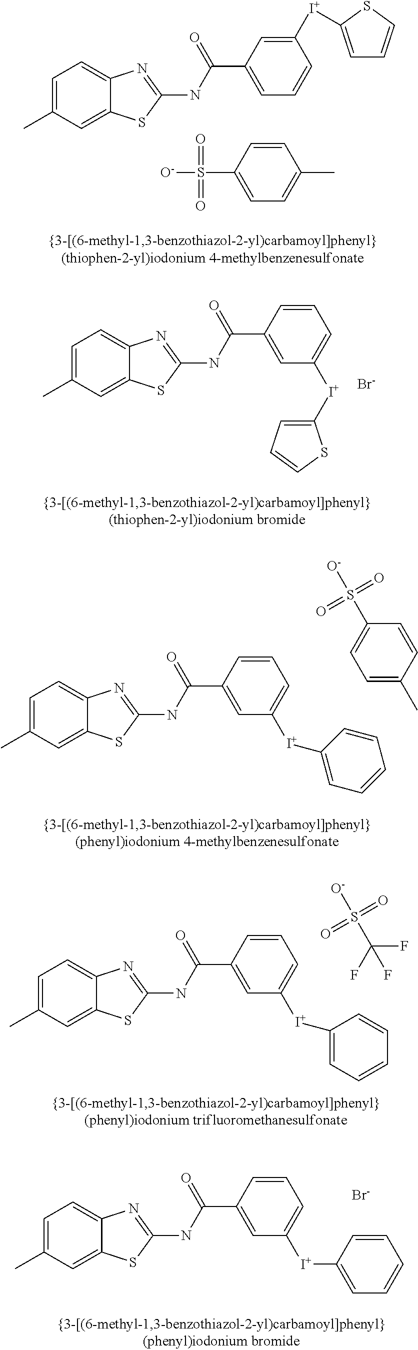

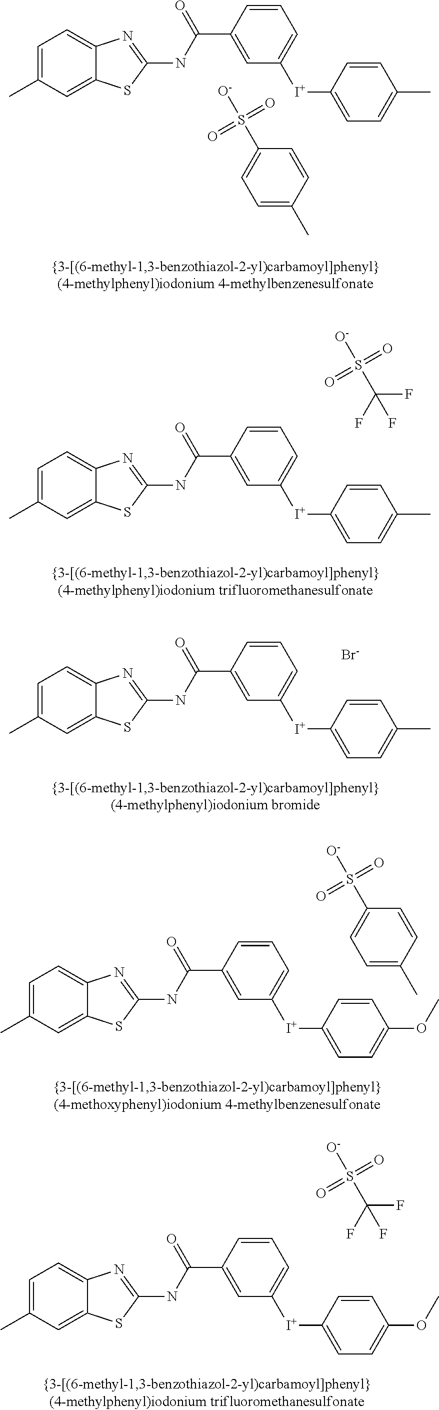

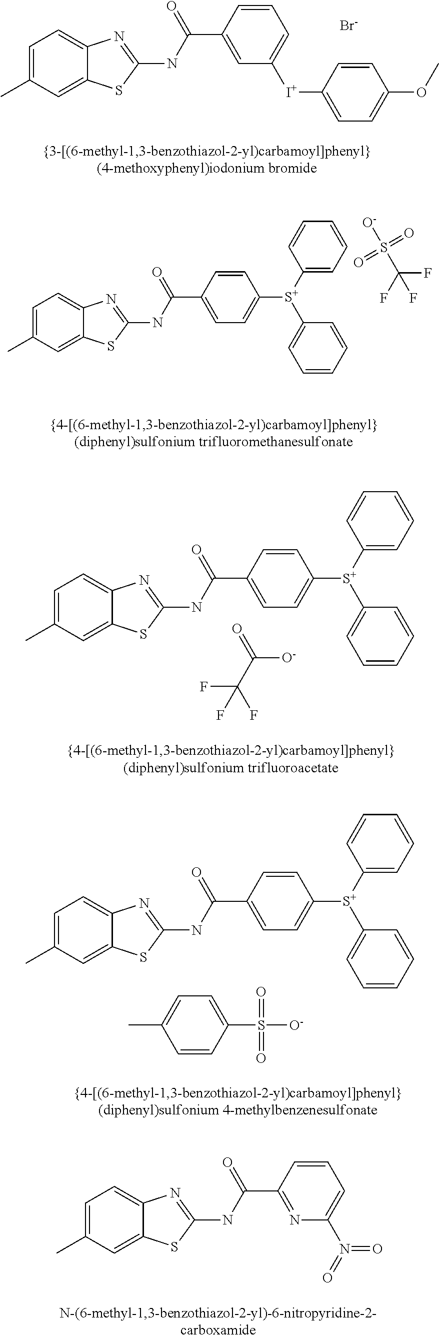

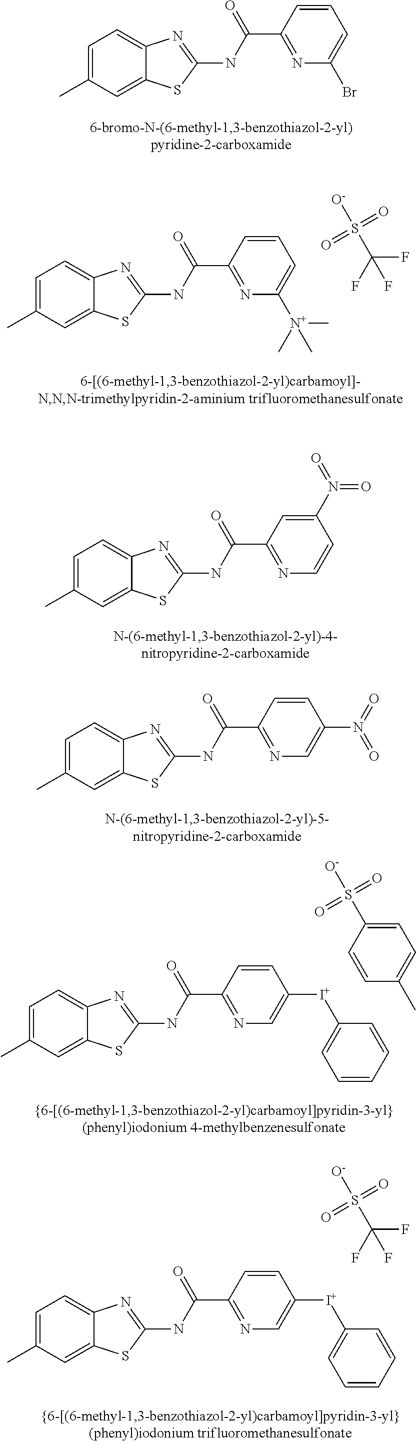

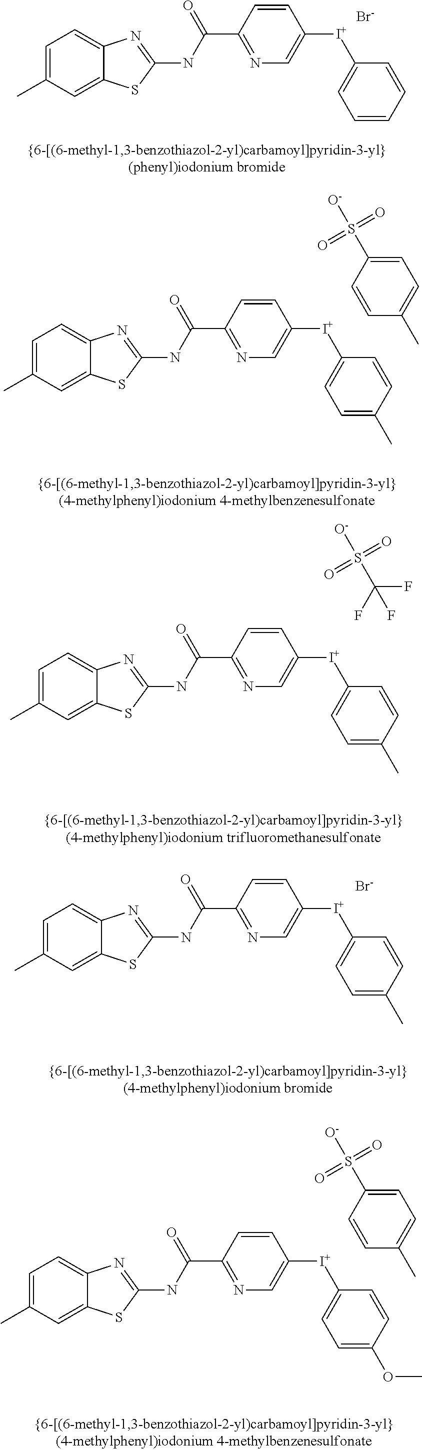

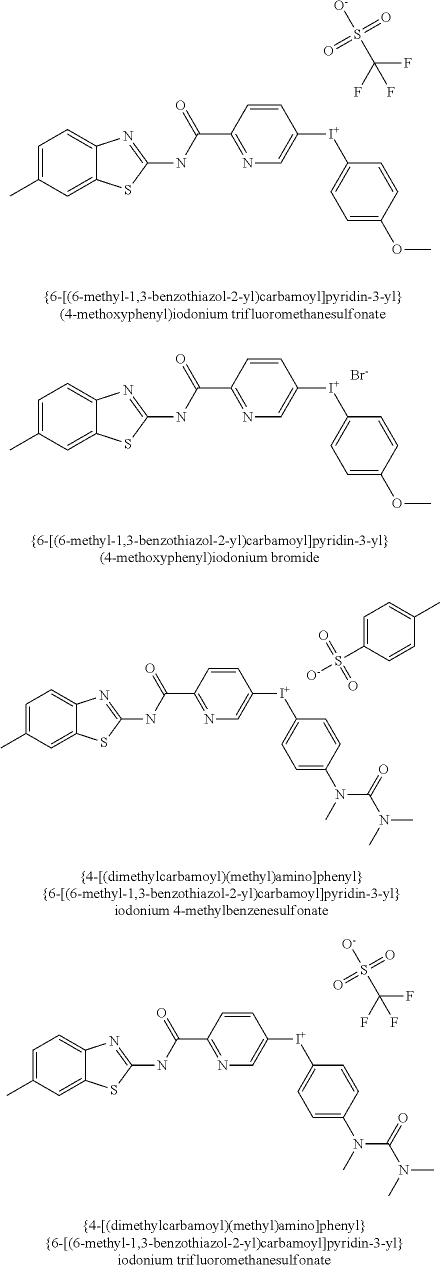

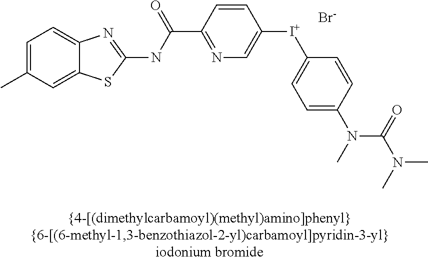

In a tenth aspect the present invention is directed to the preparation of ionic “precursor compounds having formula I” to which is added the corresponding acid HX of the corresponding counter ion X⁻_with a 0.01 to 50 weight percentage in their preparation;

in one embodiment the preparation of ionic “precursor compounds having formula I” comprises the acid HX of the corresponding counterion X⁻ with a content of 1 to 40 weight percentage;
in another embodiment the preparation of ionic “precursor compounds having formula I” comprises the acid HX of the corresponding counterion X⁻ with a content of 5 to 35 weight percentage;
in another embodiment the preparation of ionic “precursor compounds having formula I” comprises the acid HX of the corresponding counterion X⁻ with a content of 5 to 20 weight percentage;
in yet another embodiment the preparation of ionic “precursor compounds having formula I” comprises the acid HX of the corresponding counterion X⁻ with a content of 10 to 35 weight percentage;
in another embodiment the preparation of ionic “precursor compounds having formula I” comprises the acid HX of the corresponding counterion X⁻ with a content of 10-15 weight percentage;
in another embodiment the preparation of ionic “precursor compounds having formula I” comprises the acid HX of the corresponding counterion X⁻ with a content of 15 to 20 weight percentage;
in another embodiment the preparation of ionic “precursor compounds having formula I” comprises the acid HX of the corresponding counterion X⁻ with a content of 20 to 25 weight percentage;
in another embodiment the preparation of ionic “precursor compounds having formula I” comprises the acid HX of the corresponding counterion X⁻ with a content of 25 to 30 weight percentage;
in another embodiment the preparation of ionic “precursor compounds having formula I” comprises the acid HX of the corresponding counterion X⁻ with a content of 30 to 35 weight percentage;
in another embodiment the preparation of ionic “precursor compounds having formula I” comprises the acid HX of the corresponding counterion X⁻ with a content of 35 to 50 weight percentage;
in a preferred embodiment the preparations of “precursor compounds having formula I” of that type are

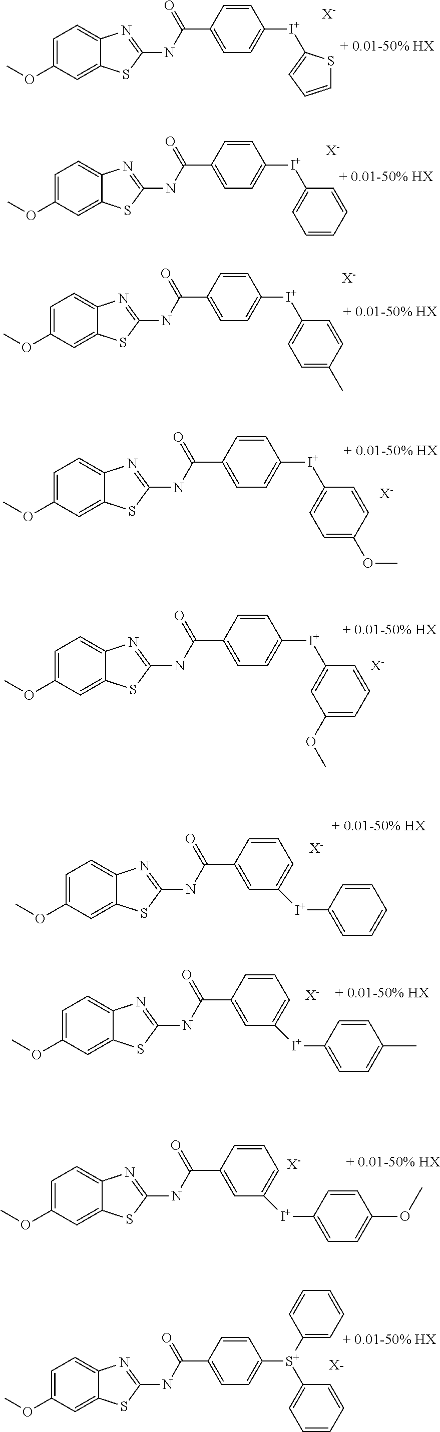

wherein HX is the corresponding acid of X- and X⁻ is defined as above;
which refers to the surprisingly made finding that synthesis yields of [¹⁸F]-compounds starting from ionic precursor compounds with a 0.01-50% counter ion acid present in their preparation can be higher than in the absence of the acid HX in the precursor;
in one embodiment the preparation of ionic “precursor compounds having formula I” comprises the counterion acid HX of corresponding counterion X⁻ whereas HX is HO—S(O)₂—C₆H₄—Me;
more preferred preparations of “precursor compound having formula I” are

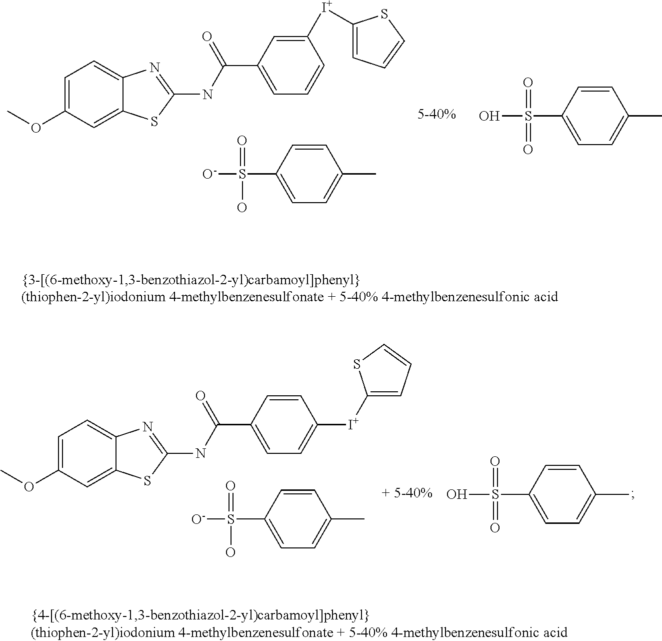

an even more preferred preparation of “precursor compound having formula I” is

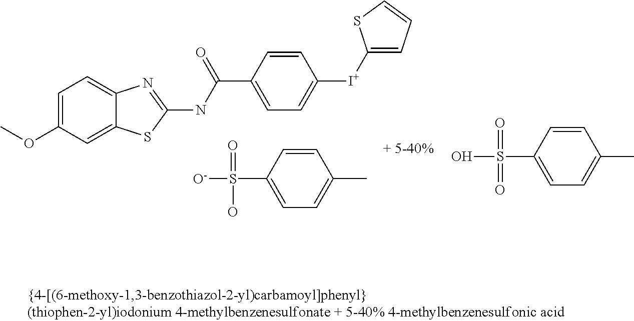

Furthermore, the invention relates to

1. A compound of formula I

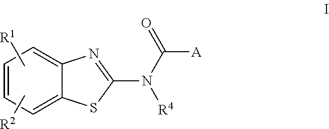

wherein
A is selected from the group comprising 1-(N—R⁹)-2,3-dihydro-1H-indol-5-yl, 1-(N—R⁹)-1H-indol-5-yl, phenyl and pyridyl, whereas A is substituted with R⁵ and R⁶.
R¹ and R² are independently and individually, at each occurrence, selected from the group comprising hydrogen, halo, cyano, trifluoromethyl, (C₁-C₅)alkyl, (C₂-C₅)alkynyl, (C₂-C₅)alkenyl, (C₁-C₅)alkoxy, (R⁷)O—, L-(CH₂—CH₂—O)ₙ—, L, L-(C₁-C₆)alkoxy, (C₁-C₅)sulfanyl and L-(C₁-C₅)sulfanyl;
R⁴ is selected from the group comprising hydrogen and (C₁-C₄)alkyl;
R⁵ and R⁶ are independently and individually, at each occurrence, selected from the group comprising hydrogen, L, L-(C₁-C₅)alkyl, L-(C₂-C₅)alkenyl, L-(C₁-C₅)alkoxy, L-(C₂-C₅)alkynyl, (C₁-C₅)sulfanyl, L-(C₁-C₅)sulfanyl, (C₁-C₅)alkyl, (C₂-C₅)alkenyl, (C₁-C₅)alkoxy, (R⁷)O—, halo, trifluoromethyl, cyano, —C(O)O—((C₁-C₅)alkyl), —N(R⁸)(L-(C₁-C₅)alkyl), —N(L-(C₁-C₄)alkyl)((C₁-C₄)alkyl), —N(R⁸)((C₁-C₄)alkyl) and —N((C₁-C₄)alkyl)₂;
L is selected from the group comprising R¹⁰, R³, F, [¹⁹F]fluoro and [¹⁸F]fluoro;
R³ is a leaving group;
R¹⁰ is selected from the group comprising R²⁰ and R³⁰;
R²⁰ is selected from the group comprising iodo, —Sn((C₁-C₆)alkyl)₃, —B(OR⁵)(OR⁶¹) and —NMe₂;
R³⁰ is hydroxy;
R⁷ is selected from the group comprising hydrogen and R¹⁷;
R⁸ is selected from the group comprising hydrogen and R¹⁸;
wherein n is an integer from 2 to 6;
including all isomeric forms of said compound, including but not limited to enantiomers and diastereoisomers as well as racemic mixtures,
and any pharmaceutically acceptable salt, ester, amide, complex or prodrug thereof;
with the proviso that compounds of Formula I contain exactly one L.
2. A compound of count 1, wherein
A is selected from the group comprising phenyl and pyrid-2-yl, whereas A is substituted with R⁵ and R⁶;
R¹ and R² are independently and individually, at each occurrence, selected from the group comprising hydrogen, fluoro, iodo, L, (C₁-C₃)alkyl and (C₁-C₃)alkoxy;
R⁴ is selected from the group comprising hydrogen and methyl;
R⁵ and R⁶ are independently and individually, at each occurrence, selected from the group comprising hydrogen, L, L-(C₁-C₃)alkoxy, methyl, bromo, fluoro, trifluoromethyl, cyano, —N(R⁸)(methyl) and —N(methyl)₂;
L is selected from the group consisting of [¹⁸F]fluoro, [¹⁹F]fluoro, or a leaving group.
3. A compound according to count 1 selected from the group consisting of compounds having the formula

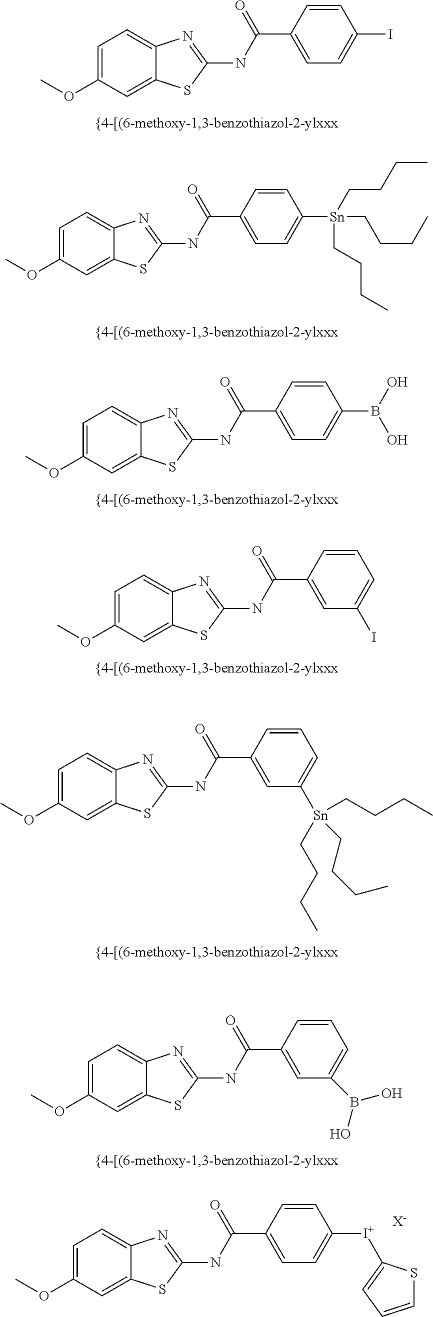

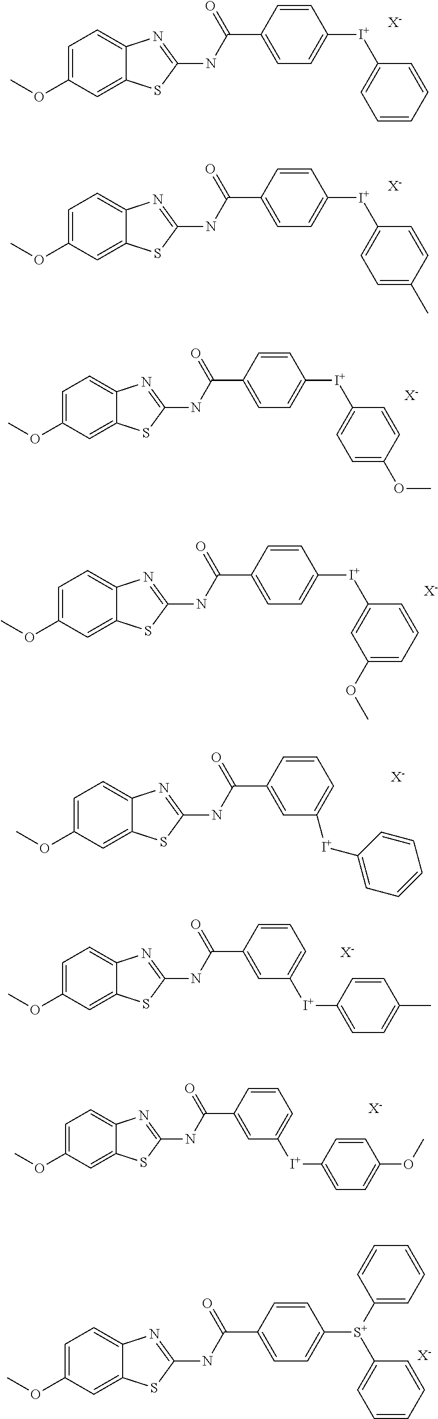

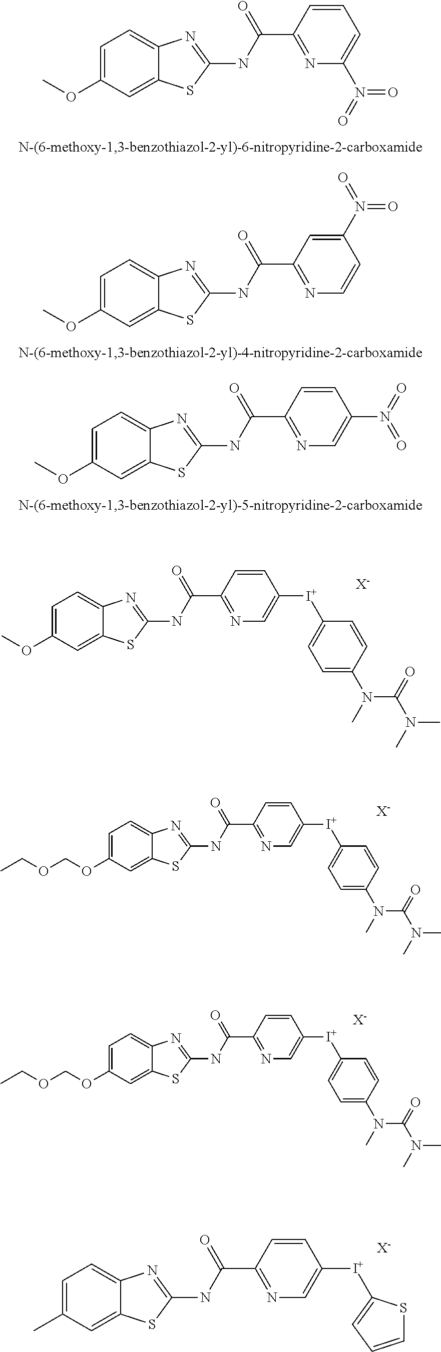

wherein X⁻ is selected from the group comprising anion of an inorganic acid and anion of an organic acid.
4. A compound according to count 1 selected from the group consisting of compounds having the formula

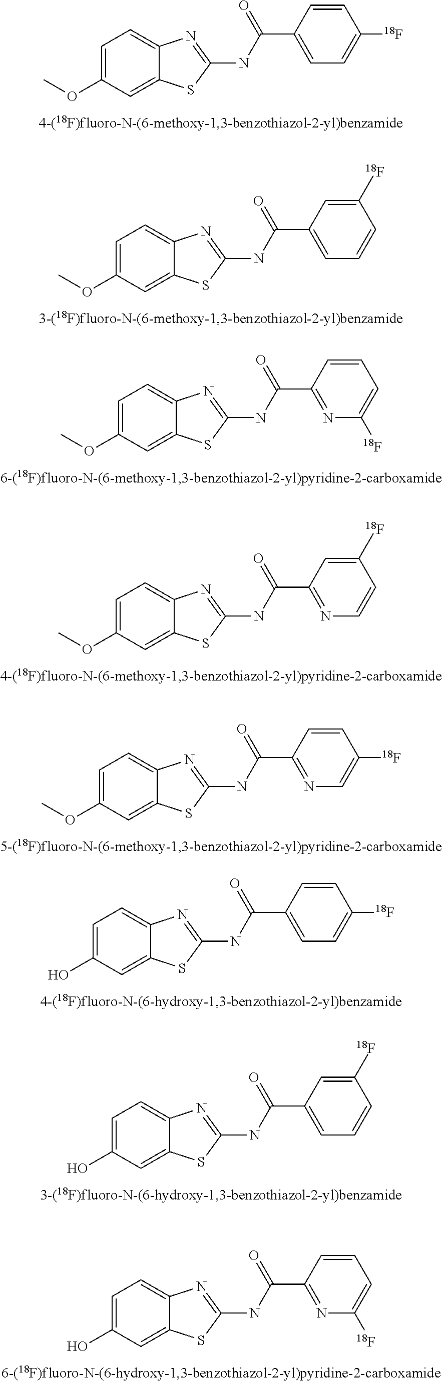

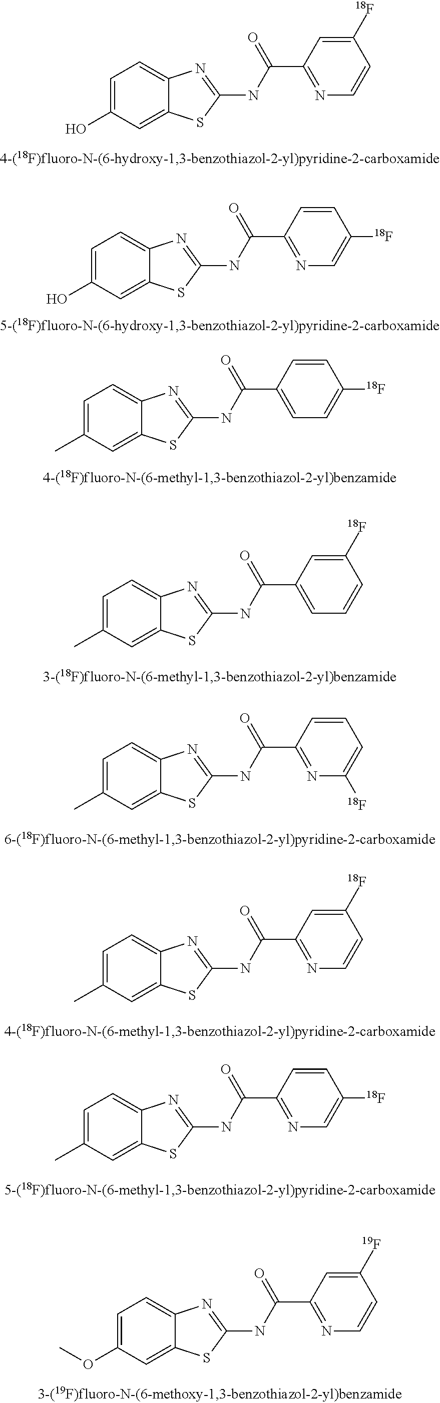

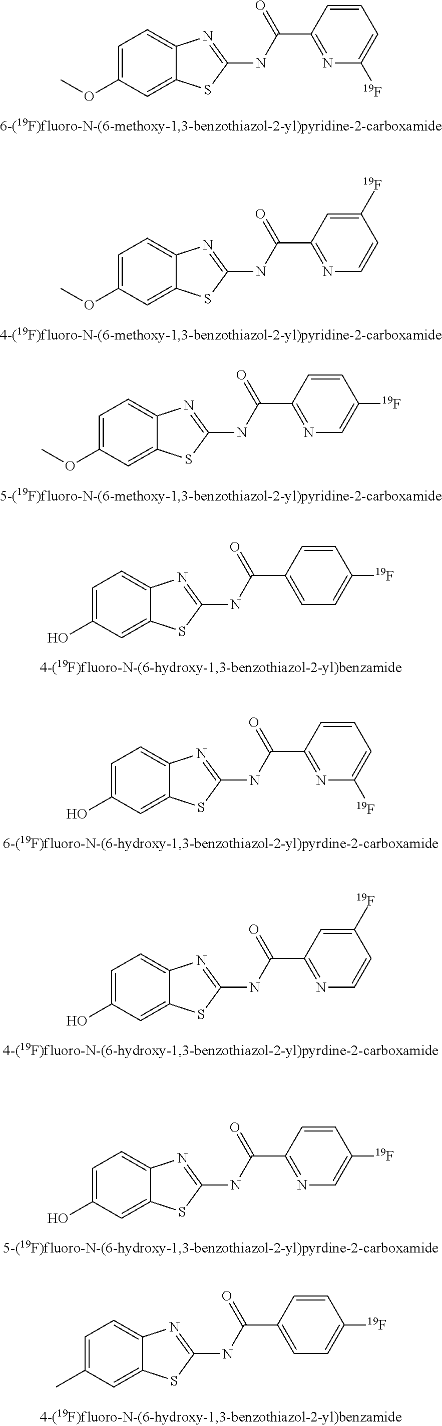

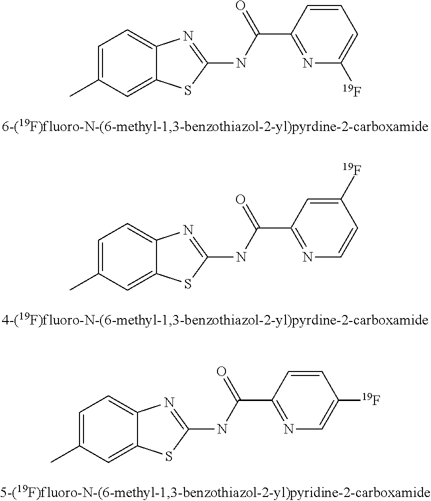

5. A compound according to count 4 selected from the group consisting of

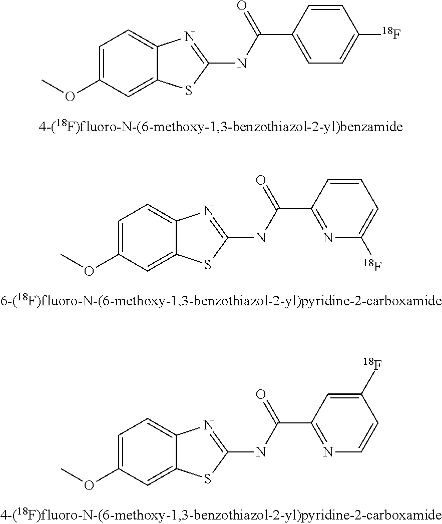

6. A radioactively labelled halogenated compound according to counts 1-2, 4 or 5 as a compound for diagnostic imaging.
7. A compound according to count 6, wherein the radioactive label is [F-18].
8. A compound according to count 6 or 7 as a compound for diagnostic imaging of a disease selected from the group consisting of Alzheimer's disease, a neurodegenerative disorder, or an amyloidosis.
9. A method for the preparation of a fluorinated compound according to counts 1, 2, 4, or 5 the method comprising reacting a suitable precursor molecule with a fluorinating agent.
10. A method for the preparation of a fluorinated compound according to count 4 or 5, the method comprising reacting a respective precursor molecule of count 3 with a fluorinating agent.
11. A method for diagnosing a disease in a mammal selected form the group consisting of Alzheimer's disease, a neurodegenerative disorder, or an amyloidosis, the method comprising administering a radioactively labelled compound of counts 1, 2, 4, or 5 to said mammal, imaging said mammal and detecting the signal.
12. The method according to count 11, wherein the compound is a [¹⁸F] labelled compound of count 4 or a compound of count 5.
13. The method of count 12, wherein said imaging is performed using a method selected from the group consisting of PET, SPECT, MR-spectroscopy, and MR-tomography.
14. A method according to counts 11-13, wherein the effect of a therapy is monitored.
15. A method of imaging amyloid plaques in a mammal, said method comprising administering a radioactively labelled compound of counts 1, 2, 4, or 5 to said mammal, imaging said mammal and detecting the signal.
16. A compound of formula VI

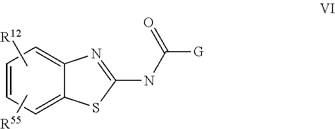

wherein
G is selected from the group comprising 1-(N—R¹¹)-2,3-dihydro-1H-indol-5-yl, 1-(N—R¹¹)-1H-indol-5-yl, phenyl and pyridyl, whereas G is substituted with R¹³ and R¹⁵.
R¹¹ is selected from the group comprising (C₁-C₄)alkyl, R¹⁸ and R¹⁴;
R¹² is selected from the group comprising hydrogen and R¹⁴—O—
R¹³ is selected from the group comprising hydrogen, (R¹⁴)O— and —N((C₁-C₄)alkyl)R¹⁴;
R¹⁴ is hydrogen:
R¹⁵ and R⁵⁵ are independently and individually selected from the group comprising hydrogen, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
R¹⁸ is a amine-protecting group;
including all isomeric forms of said compound, including but not limited to enantiomers and diastereoisomers as well as racemic mixtures,
and any pharmaceutically acceptable salt, ester, amide, complex or prodrug thereof;
with the proviso that compounds of formula IV contain exactly one R¹⁴.
17. A method of preparation of compounds of Formula Ib,

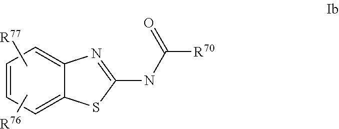

said method comprising the steps:
F-fluorinating a compound of formula V

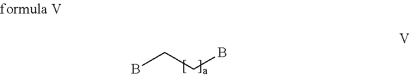

with an F-fluorinating agent to yield a compound of formula IV,

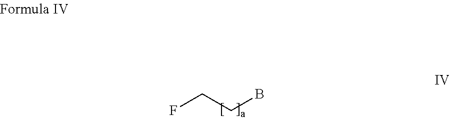

substituting said compound of formula IV with a compound of formula VI

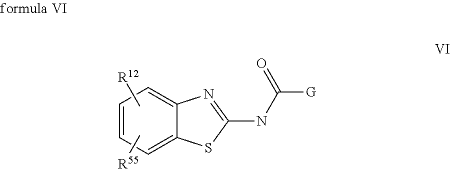

deprotection in those cases where compounds of formula VI comprise R¹⁸ or R¹⁷;
   wherein
   R⁷⁰ is selected from the group comprising 1-(N—R⁷¹)-2,3-dihydro-1H-indol-5-yl, 1-(N—R⁷¹)-1H-indol-5-yl, phenyl and pyridyl, whereas R⁷⁰ is substituted with R⁷³ and R⁷⁵;
   R⁷¹ is selected from the group comprising (C₁-C₄)alkyl, hydrogen, R¹⁸ and (L-CH₂—(CH₂)ₐ)—;
   R⁷³ is selected from the group comprising hydrogen, (L-CH₂—(CH₂)ₐ—)O—, —N(L-CH₂—(CH₂)ₐ—)(H) and —N((C₁-C₄)alkyl)(L-CH₂—(CH₂)₂—);
   R⁷⁵ and R⁷⁶ are independently and individually selected from the group comprising hydrogen, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
   R⁷⁷ is selected from the group comprising hydrogen and (L-CH₂—(CH₂)ₐ)—O;
   wherein L in Formula Ib is [¹⁸F]fluoro or [¹⁹F]fluoro, with the proviso that compounds of Formula Ib comprise exactly one L;
   F in Formula IV is [¹⁸F]fluoro or [¹⁹F]fluoro;
   a is an integer from 0 to 5;
   B is a leaving group;
   G is selected from the group comprising 1-(N—R¹¹)-2,3-dihydro-1H-indol-5-yl, 1-(N—R¹¹)-1H—I;dol-5-yl, phenyl and pyridyl, whereas G is substituted with R¹³ and R¹⁵;
   R¹¹ is selected from the group comprising (C₁-C₄)alkyl, R¹⁸ and R¹⁴;
   R¹² is selected from the group comprising hydrogen and (R¹⁴)O—;
   R¹³ is selected from the group comprising hydrogen, (R¹⁴)O—, —N(R¹⁴)(R¹⁸) and N((C₁-C₄)alkyl)(R¹⁴);
   is R¹⁴ is hydrogen;
   R¹⁵ and R⁵⁵ are independently and individually selected from the group comprising hydrogen, (R¹⁷)O—, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
   R¹⁷ is a phenol protecting group;
   R¹⁸ is a amine-protecting group;
   including all isomeric forms of said compound, including but not limited to enantiomers and diastereoisomers as well as racemic mixtures,
   and any pharmaceutically acceptable salt, ester, amide, complex or prodrug thereof;
   wherein said F-fluorinating agent is as defined above,
   and wherein F=¹⁸F or ¹⁹F,
   with the proviso that compounds of formula VI contain exactly one R¹⁴.
   18. A method of preparation of compounds of Formula Ic,

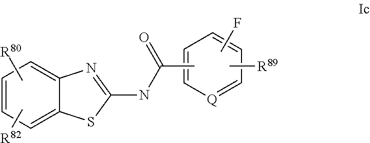

comprises the step:
F-fluorinating a compound of formula XV

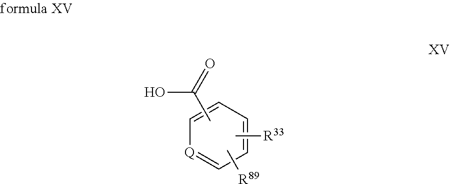

with an F-fluorinating agent to yield a compound of formula XIV,

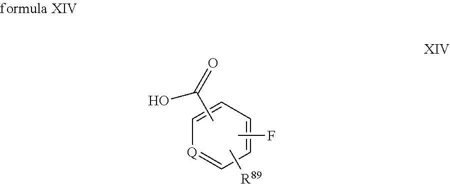

coupling said compound of formula XIV (or an activated derivative (e.g. active ester) of said compound of Formula XIV) with a compound of formula XVI

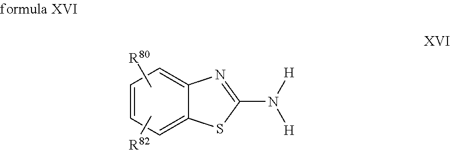

wherein F in Formula XIV and in Formula Ic is selected from the group comprising [¹⁸F]fluoro and [¹⁹F]fluoro;
Q is selected from the group comprising nitrogen and C(H);
R³³ is selected from the group comprising —I⁺(R²⁵)(X⁻), —I⁺(R²⁶)(X⁻), nitro, —N⁺(Me)₃(X⁻), —S⁺(R²⁵)(R²⁵)(X⁻), —S⁺(R²⁵)(R²⁶)(X⁻), —S⁺(R²⁶)(R²⁶)(X⁻), chloro and bromo;
R⁸⁹ is selected from the group comprising hydrogen, (C₁-C₅)alkyl, (C₂-C₅)alkenyl, (C₁-C₅)alkoxy, halo, trifluoromethyl, cyano, —C(O)O—((C₁-C₅)alkyl), —N(R¹⁸)((C₁-C₄)alkyl) and —N((C₁-C₄)alkyl)₂;
R¹⁸ is a amine-protecting group;
R⁸⁰ and R⁸² are independently and individually, at each occurrence, selected from the group comprising hydrogen, halo, cyano, trifluoromethyl, (C₁-C₅)alkyl, (C₂-C₅)alkynyl, (C₂-C₅)alkenyl, (C₁-C₅)alkoxy and (R¹⁷)O—;
R¹⁷ is a phenol protecting group;
X⁻ is selected from the group comprising anion of an inorganic acid and anion of an organic acid;
R²⁵ is aryl and
R²⁶ is heteroaryl.
19. A kit, comprising a compound according to counts 1-5, or 16.

### BRIEF DESCRIPTION OF THE FIGURES

FIG. 1: Brain uptake of compound 1f in % of injected dose per gram tissue [% ID/g]. Distribution of F-18 signal after administration of compound 1f in mice at 2 min and 30 min.

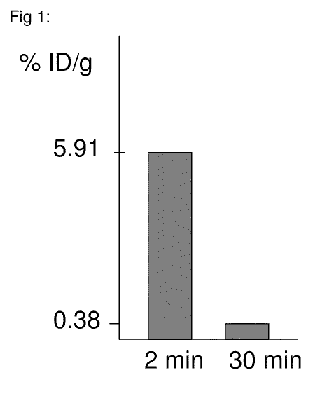

FIG. 2: Autoradiographical analysis of binding of compound 1f to cryosections from cortex of Alzheimer's disease patients (AD) and controls without Aβ plaques (HC/FTD) (healthy control/frontotemporal dementia). Specific binding in plaque-rich regions of AD samples is indicated by arrows.

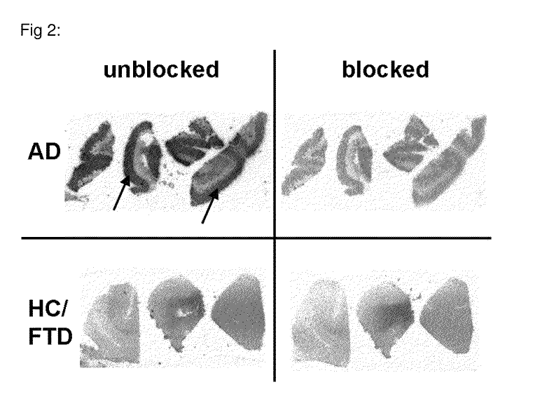

FIG. 3: Brain uptake of F-18 signal after administration of compound 3c in mice in % of injected dose per gram tissue [% ID/g] at 2 min and 30 min.

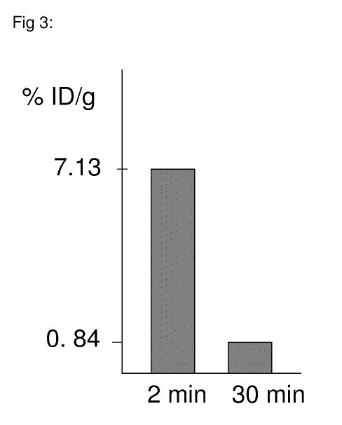

FIG. 4: Autoradiographical analysis of binding of compound 3c to paraffine-sections from cortex of Alzheimer's disease patients (AD) and controls without Aβ plaques (HC/FTD) (healthy control/frontotemporal dementia). Specific binding in plaque-rich regions of AD samples is indicated by arrows.

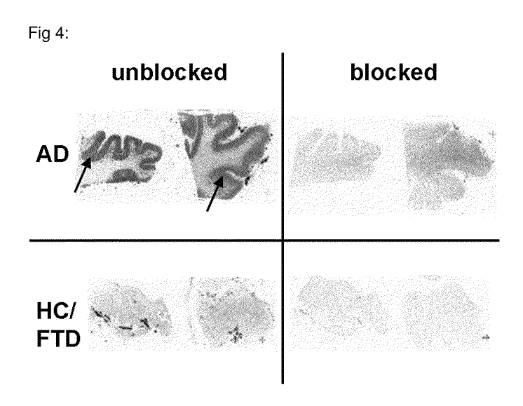

FIG. 5: Brain uptake of F-18 signal after administration of compound 2c in mice in % of injected dose per gram tissue [% ID/g] at 2 min and 30 min.

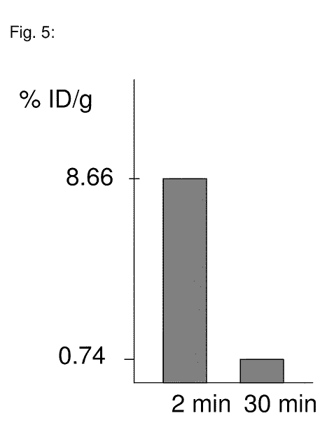

FIG. 6: Autoradiographical analysis of binding of compound 2c to cryosections from cortex of Alzheimer's disease patients (AD) and controls (HC/FTD) without Aβ plaques (healthy control/frontotemporal dementia). Specific binding in plaque-rich regions of AD samples is indicated by arrows.

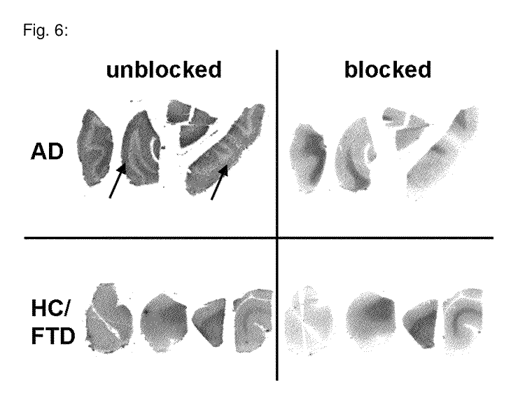

FIG. 7: 1050 values in [nM] of selected compounds measured in a competition assay using brain homogenate from AD patients.

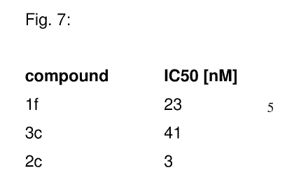

FIG. 8: ratio of 2 min to 30 min uptake value [% ID/g] in mice brain for compounds 1f, 2c and 3c.

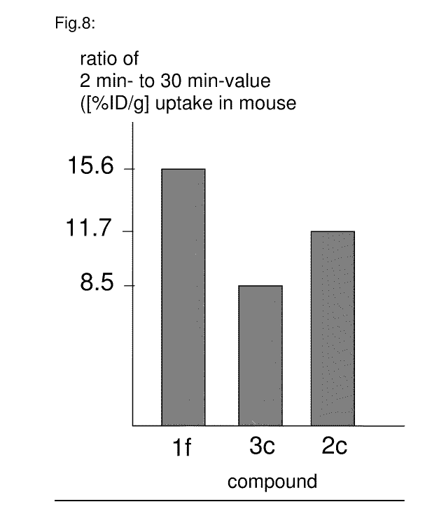

FIG. 9: Preparative HPLC chromatogram [¹⁸F]SFB (compare example 1f (method 2)) (gamma-detection).

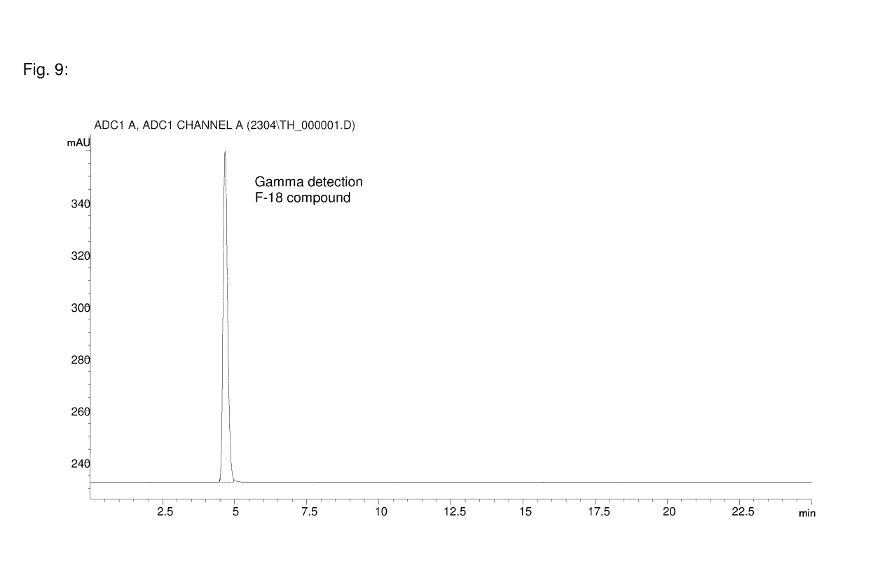

FIG. 10: Preparative HPLC chromatogram of example if (method 2) (gamma-detection).

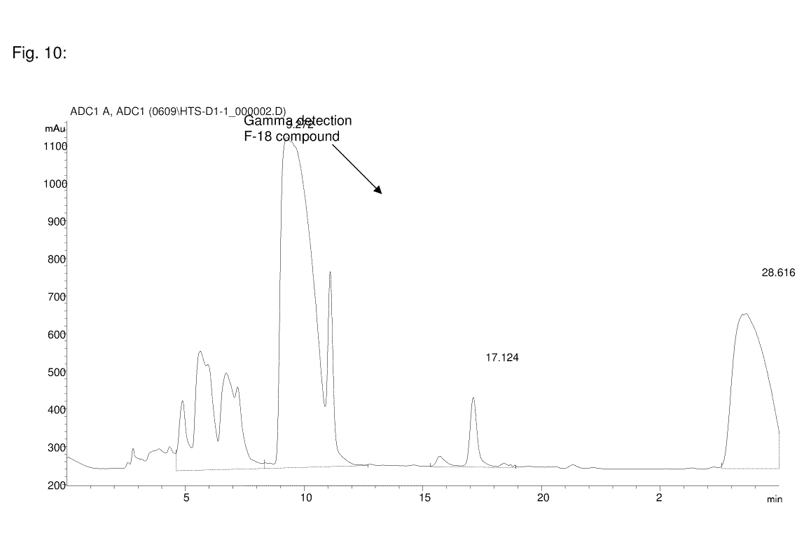

FIG. 11: Analytical HPLC chromatogram of example 1f (method 2) (gamma-detection).

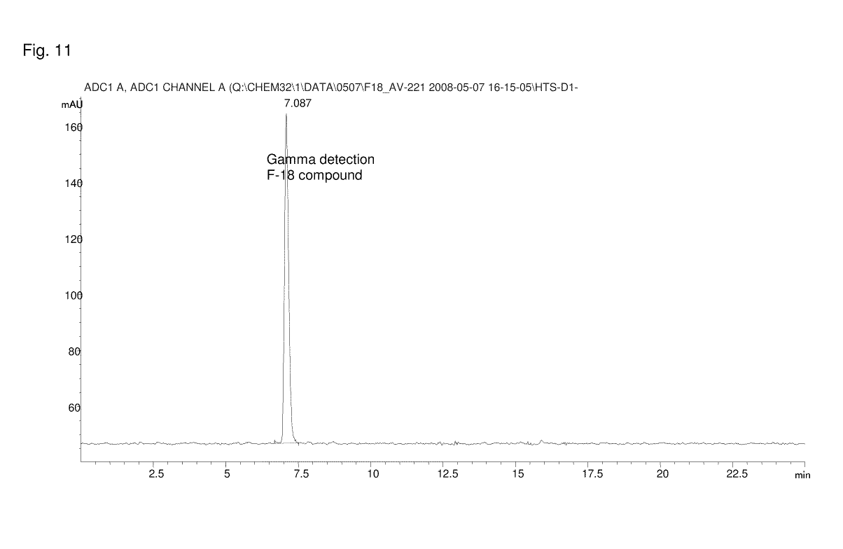

FIG. 12: Analytical HPLC chromatogram of example 1a UV detection (method 2).

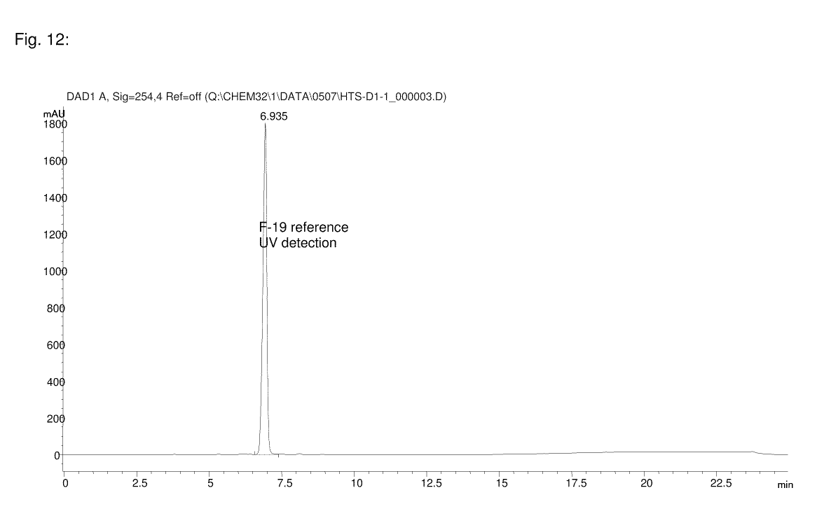

FIG. 13.: Analytical HPLC chromatogram of example 2c (gamma detection).

FIG. 14: Analytical HPLC chromatogram of example 2b (UV Detection).

FIG. 15: Analytical HPLC chromatogram of example 3c (Gamma Detection).

FIG. 16: Analytical HPLC chromatogram of example 3b (UV Detection).

FIG. 17: Analytical HPLC chromatogram of example 1f (method 1, Gamma Detection).

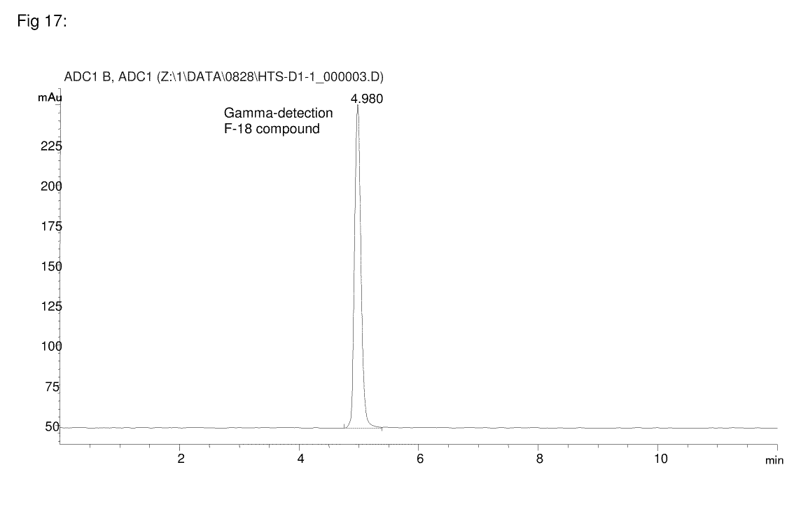

FIG. 18: Analytical HPLC chromatogram of example 1a (method 1, UV Detection).

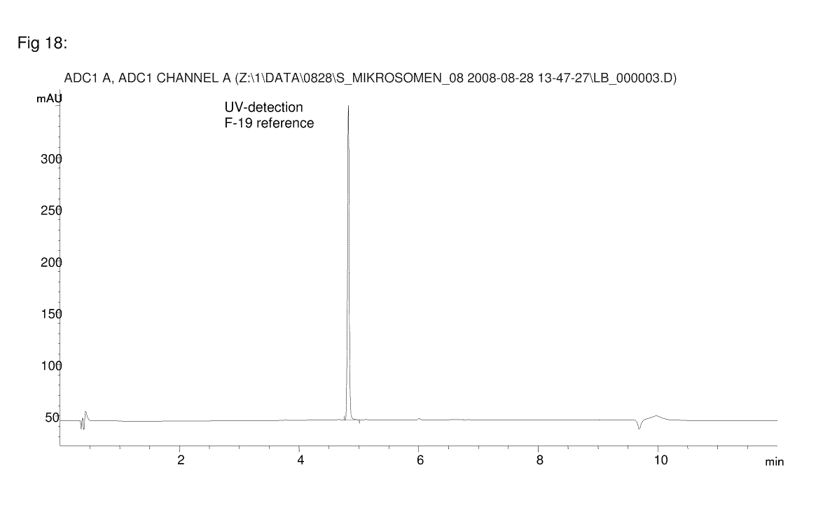

### EXPERIMENTAL SECTION

### Biological Data

### Methods

### Binding Studies Using Human Brain Homogenate

A competition assay with a tritiated amyloid ligand was performed in 96-well plates (Greiner bio-one; Cat. 651201; Lot. 06260130) using brain homogenate from AD patients.

Homogenates were prepared by homogenizing (Ultra-Turrax, setting 2, 30 s, 24000 rpm) dissected frontal cortex containing grey matter and white matter from AD patients in phosphate buffered saline (PBS, pH 7.4). The homogenate with a concentration of 100 mg wet tissue/ml was divided into aliquots of 300 μl and stored at −80° C.

Varying concentrations of the unlabeled test substances were incubated with 100 μg/ml homogenate and 10 nM of the tritiated ligand in PBS, 0.1% BSA (final volume 200 μl) for 3 h at room temperature. Subsequently the binding mixture was filtered through Whatman CF/B filters (wetted with PBS, 0.1% BSA) using a Filtermate 196 harvester (Packard). Filters were then washed twice with PBS, 0.1% BSA and 40 μl scintillator was added to each well before the bound radioactivity was measured in a TopCount devise (Perkin Elmer). Non-specific binding was assessed by adding an access of 1000× of the tritiated ligand to the reaction mixture. Finally IC50 values were calculated with the help of appropriate analysis software:

### Autoradiographical Analysis

Fresh frozen as well as paraffin embedded sections of the frontal lobe from Alzheimer's dementia patients, frontotemporal dementia patients and age matched controls were used for the study.

Frozen sections, sliced at 18 μm thickness on a cryostate (Leica, Germany) and paraffin sections, sliced on a sliding microtom (Leica) at a thickness of 6 μm, were mounted onto glass slides (Superfrost Plus, Fa.Menzel, Braunschweig Germany). Frozen sections were allowed to adhere to the slides for several nights at −20° C. The paraffin sections were deparaffinized using routine histological methods. For binding studies sections were incubated with the F-18 labeled test compound at 10 Bq/μl diluted in 25 mM Hepes buffer, pH 7.4, 0.1% (BSA) (200-300 μl/slide) for 1.5 hour at room temperature in a humidified chamber. For blocking experiments a 1000-fold access of the unlabeled test substance was added to the incubation mixture. After hybridization, sections were washed four times with Hepes buffer, 0.1% BSA (or alternatively two times with 40% ethanol) and finally dipped two times into dest. water for 10 sec. The air-dried sections were exposed to imaging plates and signals were detected by a phosphoimager device (Fuji BAS5000).

### Biodistribution

Biodistribution and excretion studies were performed in male NMRI mice (body weight app. 30 g; 3 animals per time point). The animals were kept under normal laboratory conditions at a temperature of 22±2° C. and a dark/light rhythm of 12 hours. Food and water were provided ad libitium. During an acclimation period of at least 3 days before the beginning of the study animals were clinically examined to ascertain the absence of abnormal clinical signs. At 2, 5, 30, 60, 240 min post intravenous injection via the tail vein of ca. 150 kBq in 100 μl of the test compound, urine and feces were quantitatively collected. At the same time points, animals were sacrificed by decapitation and under isoflurane anaesthesia and the following organs and tissues were removed for the determination of radioactivity using a gamma-counter: spleen, liver, kidney, lung, femur, heart, brain, fat, thyroid, muscle, skin, blood, tail, stomach (without content), testicle, intestine (with content), pancreas, adrenals, and the remaining body. For analysis the decay corrected percentage of the injected dose per tissue weight (% ID/g±standard deviation) was calculated.

### General Chemical Procedures

### 1: Amide formation for carboxylic acid derivatives and derivatives of 1,3-benzothiazol-2-amine using TBCR as condensating agent

To a solution of 1.3 eq. carboxylic acid in DMF (4.3 ml/mmol carboxylic acid) is added 1.3 eq. 4-(4,6-dimethoxy-1,3,5-triazin-2-yl)-4-methylmorpholin-4-ium tetrafluoroborate (TBCR (J. Am. Chem. Soc. 2005, 127, 16912-16920)) and 1.95 eq. N-methyl morpholine. The reaction mixture is stirred for 40 min. 1 eq. amine in DMF (1.5 ml/mmol) is added drop by drop. The reaction mixture is stirred between 4 hours to 20 hours. The reaction mixture is reduced by evaporation. A portion of the crude product is dissolved in DMSO and the desired product is purified by preparative HPLC and subsequent lyophilisation of the corresponding HPLC fraction.

### 2: Fluorination with Non-Radioactive [F-19]Fluoride

To a solution of 1 eq. starting material in acetonitrile (2 ml/eq.) 1.1 eq. potassium fluoride and kryptofix (1.1 eq.) are added. The reaction mixture is heated by microwave (130° C., 15 min) and cooled to room temperature again. The reaction mixture is diluted with 10 ml diethyl ether and 10 ml water. The organic phase is separated. The aqueous phase is extracted three times with 10 ml diethyl ether. The combined organic phases are washed with brine and dried with magnesium sulfate. The solvent is evaporated and the residue is purified by column chromatography with ethyl acetate-hexane gradient.

### 3: Fluorination with Radioactive [F-18]Fluoride

Aqueous [¹⁸F]Fluoride (0.1-5 GBq) is trapped on a QMA cartridge and eluted with 5 mg K2.2.2 in 0.95 ml acetonitrile+1 mg potassium carbonate in 50 μl water into a Wheaton vial (5 ml). The solvent is removed by heating at 120° C. for 10 mins under a stream of nitrogen. Anhydrous acetonitrile (1 ml) is added and evaporated as before. This step is repeated three times. A solution of starting material (1 mg) in 300 μl anhydrous DMF is added. After heating at 120° C. for 10 min the crude reaction mixture is analyzed using analytical HPLC: ACE3-C18 50 mm×4.6 mm; solvent gradient: start 5% acetonitril-95% acetonitril in water in 7 min., flow: 2 ml/min. The desired F-18 labeled product is confirmed by co-injection with the corresponding non-radioactive F-19 fluoro-standard on the analytical HPLC. The crude product is pre-purified via a C18 SPE cartridge and (50-2500 MBq) of that pre-purified product are purified by preparative HPLC: ACE 5-C18-HL 250 mm×10 mm; 62% isocratic acetonitrile in water 25 min., flow: 3 ml/min The desired product is obtained (30-2000 MBq) as reconfirmed by co-injection with the non-radioactive F-19 fluoro standard on the analytical HPLC. The sample is diluted with 60 ml water and immobilized on a Chromafix C18 (S) cartridge, which is washed with 5 ml water and eluted with 1 ml ethanol to deliver 20-1800 MBq product in 1000 μl ethanol.

### 4: Alkylation of Phenols

To a stirred solution of 1 eq. starting material (phenol derivative) and 1.5 eq. potassium carbonate in dimethyl formamide 3 ml/1 eq. is added 2.5 mmol alkylating agent. The reaction mixture is heated at 70° C. for 6 hours or by microwave to 110° C. for 15 min. The solvent of the reaction mixture is evaporated. Water and methyl tert-butyl ether are added. The organic phase is separated. The aqueous phase is extracted three times with methyl tert-butyl ether diethyl ether. The combined organic phases are washed with water, brine and dried with magnesium sulfate. The solvent is evaporated and the residue is purified by column chromatography with ethyl acetate-hexane gradient.

### 5: Conversion of Alcohol to Corresponding O-Sulfonate

To a solution of 1 eq. starting material and 1.5 eq. diisopropyl ethyl amine in 3 ml/mmol dichloromethane is added 1.3 eq. mesyl chloride in some dichloromethane drop wisely at −10° C. The stirred reaction mixture is warmed over a period of 4.5 h to room temperature and diluted with dichloromethane.

The organic phase is washed with saturated sodium hydrogen carbonate solution, water and brine. The organic phase is dried with magnesium sulfate. The crude product is purified by silica column chromatography (ethyl acetate-hexane gradient).

### 6: Conversion of Alcohol to Corresponding O-Sulfonate (Version 2)

To a solution of 1 eq. starting material in dichloromethane (1.4 ml/eq.) and pyridine (1.4 ml/eq.) pyridine is added (1.1 eq.) aryl sulfonyl chloride in dichloromethane (1 ml/eq.) drop wisely at −10° C. The stirred reaction mixture is warmed over a period of 4.5 h to room temperature and diluted with dichloromethane. The organic phase is washed with 0.25 N sulfuric acid (three times), saturated sodium hydrogen carbonate solution, water and brine. The organic phase is dried with magnesium sulfate. The crude product is purified by silica column chromatography (ethyl acetate-hexane gradient).

### 7: Heterogeneous Hydrogenation

To a stirred solution of ca. 20-50 mg palladium on Goal (10%)) isopropanol (8 ml per 1 mmol starting material) benzyl ether (educt) were added in some iso-propanol. The reaction mixture is stirred at hydrogen atmosphere for 16-20 hours. The reaction mixture is filtered; and the solvent is evaporated. The residue is purified by column chromatography with ethyl acetate-hexane gradient.

### 8: Hydrogenation with Iron

To a stirred solution of 1 eq. starting material (nitro derivative) and 5 eq. iron powder in ethanol (˜86 eq) 1 ml/eq. hydrochloric acid (37% aqueous solution) is added. The solution is refluxed for 1 hour. The solution is cooled to 0° C. 1N NaOH (40 ml/mmol starting material) is added drop wisely. Dichloromethane and brine are added. The organic phase is separated. The aqueous solution is extracted trice with dichloromethane. The combined organic phases are washed with brine and dried with magnesium sulfate. The solvent is evaporated. The residue is purified by column chromatography with ethyl acetate-hexane gradient.

### 9: Reductive Amination and Subsequent Acetylation

A stirred solution of aldehyde (1 eq.) and amine (1 eq.) in 60 ml dichloroethane (pH=5) is adjusted with glacial acetic acid to pH=5. To this solution is added 70 mmol sodium tris-acetoxy hydro borane. The reaction mixture is stirred over night and diluted with 5 ml water. The pH value is adjusted with aqueous sodium hydroxide solution to pH=8-9. The mixture is extracted three times with dichloromethane. The combined organic phases were washed with water and brine and were dried with magnesium sulfate. The desired crude product is obtained after evaporation. The crude product is diluted in dry pyridine (1.3 ml/mmol starting material) and is cooled to 0° C. To this stirred solution is added 1.25 eq. acetic acid anhydride drop by drop. The reaction mixture is stirred over night and reduced to a third of its volume and diluted with dichloromethane (2 ml/mmol) and water (2 ml/mmol). The aqueous phase is extracted three times with dichloromethane. The combined organic phases are washed with brine and dried with magnesium sulfate. The solvent is evaporated and the residue is purified by column chromatography with ethyl acetate-hexane gradient.

### 10: Deprotection of THP Ether

0.15 eq. PPTS is added to a solution of 1 eq. tetrahydropyranyl ether in 7 ml/mmol methanol. The reaction mixture is stirred over night and poured onto a stirred solution of ice-water and tert-butyl methyl ether. The organic phase is separated. The aqueous phase is extracted three times with tert-butyl methyl ether. The combined organic phases are washed with diluted sodium hydrogen carbonate, brine and dried with magnesium sulfate. The solvent is evaporated and the residue is purified by column chromatography with ethyl acetate-hexane gradient.

### 11: Amide formation using carboxylic acid chloride derivatives and derivatives of 1,3-benzothiazol-2-amine

To a solution of 1 eq. 1,3-benzothiazol-2-amine derivative in toluene (2.5 ml/1 mmol amine) is added 1.5 eq. carboxylic acid chloride derivative. The reaction mixture is refluxed for 4 h, cooled to room temperature and diluted with ethanol. A solid is obtained by filtration. The solid is washed with ethanol.

Purification method 1: A portion of the crude product is dissolved in DMSO and the desired product is purified by preparative HPLC and subsequent lyophilisation of the corresponding HPLC fraction.
Purification method 2: The solid is suspended in 0.5N NaOH solution. The solid is filtrated and treated three more times with 0.5N NaOH solution. The solid is washed with DMF and methanol (twice). The solid is dried in oil pump vacuum.

### 12: Formation of (thiophen-2-yl)iodonium 4-methylbenzenesulfonate derivatives from corresponding iodides

To a solution of 0.44 eq. iodo derivative (starting material) in dichloromethane (9 ml/1 mmol iodo derivative) and 1,1,1-trifluoro-ethanol (9 ml/1 mmol iodo derivative) is added 1 eq. meta chloro perbenzoic acid, 1 eq. thiophene and 1. eq. toluene sulphonic acid mono hydrate. The reaction mixture is stirred for 20 h. The reaction mixture is evaporated to dryness. The crude product is purified by preparative HPLC and subsequent lyophilisation of the corresponding HPLC fraction.

13: Conversion of Alcohols Towards Fluorides with DAST

To a stirred solution of 1 eq. alcohol in 60 eq. dichloromethane is added 1.5 eq. DAST drop by drop at 0° C. The reaction mixture is stirred at room temperature for 2 hours. Saturated sodium hydrogen carbonate solution is added. the mixture is stirred vigorously for 20 min. Water and dichlormethane are added. The organic phase is separated. The aqueous phase is extracted with dichloromethane. The combined organic phases are washed with brine, dried with magnesium sulphate and reduced in vacuum. The crude product is purified by chromatography.

### 14: Deprotection of Boc-Protecting Group

A solution of 1 eq. starting material in wet trifluoro acetic acid-dichloromethane mixture (1:1; ca. 10 ml/g starting material) is stirred for 4-7 hours. The reaction mixture is evaporated. The residue is solved in dichloromethane and the solution is evaporated again. The last step is repeated three times. The residue is purified by column chromatography (dichloromethane-pentane gradient, amino phase).

### Example 1

### a) Synthesis of 4-fluoro-N-(6-methoxy-1,3-benzothiazol-2-yl)benzamide (1a)

The desired product 1a (587 mg) was obtained from 1.8 g of 6-methoxy-1,3-benzothiazol-2-amine and 4-fluorobenzoyl chloride according to general procedure 11 and purification method 1.

UPLC-MS (ESI): 303 (M⁺+1, 100).

### b) Synthesis of (4-carboxyphenyl)(diphenyl)sulfonium trifluoromethanesulfonate (1b)

To a solution of 10 g (40.3 mmol) 4-iodo benzoic acid in 150 ml THF was added 1.77 g (44.35 mmol) sodium hydride in one portion. The solution was stirred for 10 min and cooled to −40° C. To this solution was added 59 ml (0.52 mM in THF) (30.83 mmol) diisopropyl magnesium bromide. The temperature was raised to −10° C. within one hour and stirred for another 2.5 h (flask A).

In another flask (flask B) 16.64 g (80.64 mmol) 1,1′-sulfinyldibenzene and 50 ml THF were stirred at −40° C. under inert and dry atmosphere. 14.6 g (80.6 mmol) trimethylsilyl trifluoromethanesulfonate were added drop by drop. The solution in flask B was stirred at −40° C. for 10 min and was added at once to the solution in flask A at −20° C. The mixture was warmed within one hour to −10° C. The reaction mixture was cooled to −70° C. and 100 ml 0.5 M hydrobromic acid solution was added to the reaction mixture. The mixture was warmed to room temperature and diluted with diethyl ether (300 ml) and 0.5M hydrobromic acid-solution (200 ml). The organic phase was separated. The aqueous phase was extracted with diethyl ether (1×200 ml) and with dichloromethane (3×200 ml). The combined organic phases were dried and evaporated. The crude product was purified by column chromatography (dichloromethane/methanol 5:1--->2:1).

UPLC-MS (ESI): 307 (M⁺, 100).

### c) {4-[(6-methoxy-1,3-benzothiazol-2-yl)carbamoyl]phenyl}(diphenyl)sulfonium trifluoromethanesulfonate (1c)

The desired product 1c (25.9 mg) was obtained from 87 mg of 6-methoxy-1,3-benzothiazol-2-amine and 1b according to general procedure 1.

UPLC-MS (ESI): 469 (M⁺, 100).

### d) 4-iodo-N-(6-methoxy-1,3-benzothiazol-2-yl)benzamide (1d)

To a stirred solution of 6-methoxy-2-benzothiazolamine (42.3 g, 235 mmol) in toluene (255 mL) was added 4-iodobenzoyi chloride (75.1 g, 282 mmol) portionwise (20-45° C. internal temperature) in an ice/water bath. After complete addition, the mixture was heated to 75° C. for 5 h. The oil bath was removed and the mixture was stirred at room temperature over night. The reaction was poured into 2.4 L of an ice/water mixture and the resulting precipitate was collected by suction filtration giving 208 g of a yellow solid.

The crude product was suspended in 1.4 L 10% aqueous sodium carbonate (2×). The suspension was filtered, washed with water (2 L) and dried (98 g).

The yellow solid was purified by repetitive dissolution in DMF (4 mL/g) at 55-75° C. and precipitation by the addition of 10% aqueous sodium carbonate (1 mL/g) followed by filtration and drying in vacuo at 45° C. to give the desired product 1d (79.0 g, 192 mmol, 81%) as a beige solid.

¹H NMR (300 MHz, DMSO-d₆) δ ppm 3.82 (s, 3H) 7.06 (dd, J=8.76, 2.54 Hz, 1H) 7.60 (d, J=2.60 Hz, 0H) 7.67 (d, J=8.85 Hz, 1H) 7.88 (dt, J=8.70, 1.90 Hz, 2H) 7.95 (dt, J=8.70, 1.90 Hz, 2H) 12.84 (br. s, 1H).

UPLC-MS (ESI): 411 (M⁺+1, 100).

### e) Synthesis of {4-[(6-methoxy-1,3-benzothiazol-2-yl)carbamoyl]phenyl}(thiophen-2-yl)iodonium 4-methylbenzenesulfonate (1e)

To a stirred suspension of 1d (33.0 g, 88.4 mmol) in dichloromethane (595 mL) and 2,2,2-trifluoroethanol (650 mL) was added 77% m-chloroperbenzoic acid (39.6 g, 177 mmol) at room temperature. After 15 min, p-toluenesulfonic acid monohydrate (34.6 g, 182 mmol) and thiophene (14.2 mL, 177 mmol) were added. After 10 h, the crude product was precipitated from the dark solution by slow addition of t-butylmethyl ether (3 L). The suspension was stirred over night and the solid (66 g) was isolated by suction filtration.

The crude product was purified by successive stirring with acetonitrile (960 mL), three times chloroform/water (640/16 mL) and chloroform (640 mL) followed by filtration to give 1e (25.3 g, 34.7 mmol, 47%, containing 7% w/w p-toluenesulfonic acid) upon drying in vacuo as a beige solid.

¹H NMR (300 MHz, DMSO-d₆) δ ppm 2.28 (s, 3H) 3.82 (s, 3H) 4.50 (br. s, 1H) 7.07 (dd, J=8.95, 2.54 Hz, 1H) 7.11 (d, J=7.91 Hz, 2H) 7.20 (dd, J=5.37, 3.86 Hz, 1H) 7.48 (d, J=8.10 Hz, 2H) 7.62 (d, J=2.64 Hz, 1H) 7.68 (d, J=8.85 Hz, 1H) 8.00 (dd, J=5.27, 1.32 Hz, 1H) 8.13 (dd, J=3.77, 1.32 Hz, 1H) 8.15 (d, J=8.67 Hz, 2H) 8.42 (d, J=8.67 Hz, 2H),

UPLC-MS (ESI): 493 (M⁺, 100).

### f) 4-(¹⁸F)fluoro-N-(6-methoxy-1,3-benzothiazol-2-yl)benzamide (1f)

### Method 1:

Aqueous [¹⁸F]Fluoride (4.2 GBq) was trapped on a QMA cartridge (Waters) and eluted with 5 mg K_{2.2.2} in 0.95 ml acetonitrile+1 mg potassium carbonate in 50 μl water into a Wheaton vial (5 ml). The solvent was removed by heating at 120° C. for 10 min under a stream of nitrogen. Anhydrous acetonitrile (1 ml) was added and evaporated as before. A solution of Thienyl-iodonium-precursor 1e (5 mg) in 500 μl anhydrous DMF was added. After heating at 130° C. for 20 min the crude reaction mixture was analyzed using analytical HPLC: ACE3-C18 50 mm×4.6 mm; solvent gradient: start 5% acetonitril-95% acetonitril in 0.1% trifluoroacetic acid in 7 min., flow: 2 ml/min. The desired F-18 labeled product was confirmed by co-injection with the corresponding non-radioactive F-19 fluoro-standard 1a on the analytical HPLC (t_{R}=4.9 min). The crude product was diluted with water and purified by preparative HPLC: ACE 5-C18-HL 250 mm×10 mm; isocratic, 45% acetonitrile in 0.1% trifluoroacetic acid, flow: 4 ml/min; t_{R}˜27 min. The desired product was obtained as reconfirmed by co-injection with the non-radioactive F-19 fluoro standard on the analytical HPLC. The collected HPLC fraction was diluted with 40 ml water and immobilized on a Sep-Pak light C18 cartridge (Waters), which was washed with 5 ml water and eluted with 1 ml ethanol to deliver 230 MBq product (10%, corrected for decay; radiochemical purity >97% (TLC)) in 1000 μl ethanol in a overall synthesis time of 90 min.

### Method 2:

[¹⁸F]SFB was synthesized according to literature [Kabalka et al., Journal of Labeled Compounds and Radiopharmaceuticals, 2008, 51, 68-71.] in a one-pot synthesis on a GE tracerlab and purified by isocratic semi preparative HPLC (t_{R}=16.5 min; 65/35 water/MeCN+0.1% TFA; ACE 5 C18-HL 250*10 mm; 5 μm; Advanced Chromatography Technologies; Cat. No.: ACE 321-2510). In a typical experiment [¹⁸F]SFB was isolated in amounts between 400 to 800 MBq after 65 min in 30-35% radiochemical yield corrected for decay. The purity was determined by HPLC to be greater than 99% (t_{R}=4.7 min; Zorbax 300SB-C18, 250*4.6 mm; 5 μm; isocratic, 50% acetonitrile in 0.1% TEA, flow: 1 ml/min). After semiprep. HPLC the volume of [¹⁸F]SFB was reduced by C-18 SPE and [¹⁸F]SFB was formulated in 2 mL acetonitrile and dried in a stream of nitrogen at 55° C. until dryness. [¹⁸F]SFB was re-dissolved in acetonitrile (200 μL) and 6-Methoxy-benzothiazol-2-ylamine (10 mg in 300 μL MeCN) was added. Incubation for 30 min at 125° C. The conjugation rate was verified by analytical HPLC (t_{R}=7.0 min; Zorbax 300SB-C18, 250*4.6 mm; 5 μm; isocratic, 50% acetonitrile in 0.1% TFA, flow: 1 ml/min). The crude product was diluted with water and purified by preparative HPLC: ACE 5-C18-HL 250 mm×10 mm; isocratic, 45% acetonitrile in 0.1% trifluoroacetic acid, flow: 4 ml/min; t_{R}=28.6 min. The desired product was obtained as reconfirmed by co-injection with the non-radioactive F-19 fluoro standard 1a on the analytical HPLC. The collected HPLC fraction was diluted with 40 ml water and immobilized on a Sep-Pak light C18 cartridge (Waters), which was washed with 5 ml water and eluted with 1 ml ethanol to deliver F-18 compound 1f in a overall radiochemical yield of 8-12%, corrected for decay; radiochemical purity >99%) in 10041 ethanol in a total synthesis time of 150 min.

### Example 2

### a) Synthesis of N-(6-methoxy-1,3-benzothiazol-2-yl)-4-nitropyridine-2-carboxamide (2a)

The desired product (2a; 40 mg) was obtained from 4-nitropyridine-2-carboxylic acid (ABCR) and 162 mg of 6-methoxy-1,3-benzothiazol-2-amine according to the general procedure 1.

¹H NMR (400 MHz, DMSO-d₆) δ ppm 3.74 (s, 3H) 6.82 (dd, 1H) 7.29 (d, 1H) 7.39 (d, 1H) 8.10 (dd, 1H) 8.84 (d, 1H) 8.92 (d, 1H)

UPLC-MS (ESI): 331 (M⁺+1, 100).

### b) Synthesis of 4-fluoro-N-(6-methoxy-1,3-benzothiazol-2-yl)pyridine-2-carboxamide (2b)

The desired product (2b; 33 mg) was obtained from 4-fluoro-pyridine-2-carboxylic acid (European Journal of Organic Chemistry; 10; (2005); 2116-2123) and 125 mg of 6-methoxy-1,3-benzothiazol-2-amine according to the general procedure 1.

¹H NMR (300 MHz, CHLOROFORM-d) δ ppm 3.89 (s, 3H) 7.06 (dd, 1H) 7.19-7.31 (m, 1H) 7.33 (d, 1H) 7.73 (d, 1H) 8.02 (dd, 1H) 8.62 (dd, 1H) 11.14 (hr. s., 1H)

UPLC-MS (ESI): 304 (M⁺+1, 100).

### c) Synthesis of 4-(¹⁸F)fluoro-N-(6-methoxy-1,3-benzothiazol-2-yl)pyridine-2-carboxamide (2c)

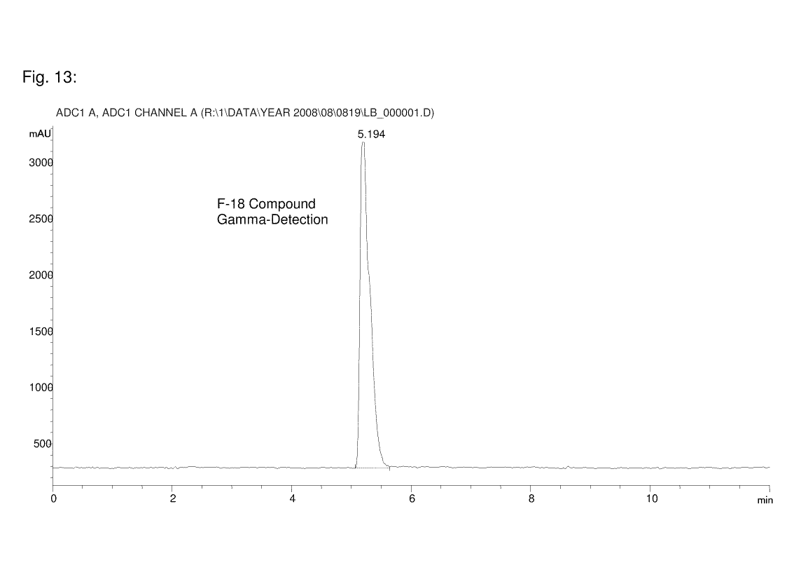

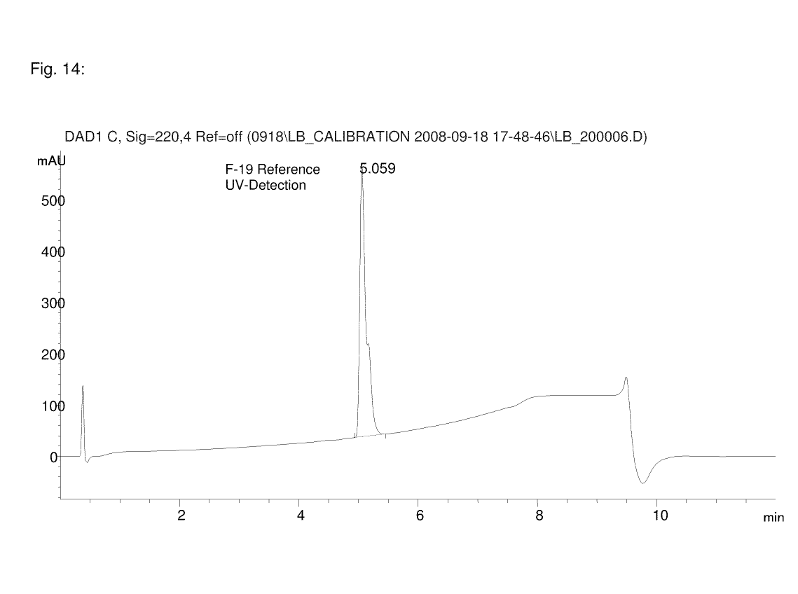

Aqueous [¹⁸F]Fluoride (6.3 GBq) was trapped on a QMA cartridge (Waters) and eluted with 5 mg K_{2.2.2} in 0.95 ml acetonitrile+1 mg potassium carbonate in 50 μl water into a Wheaton vial (5 ml). The solvent was removed by heating at 120° C. for 10 min under a stream of nitrogen. Anhydrous acetonitrile (1 ml) was added and evaporated as before. A solution of the NO₂-precursor 2a (5 mg) in 500 μl anhydrous DMSO was added. After heating at 180° C. for 20 min the crude reaction mixture was analyzed using analytical HPLC: ACE3-C18 50 mm×4.6 mm; solvent gradient: start 5% acetonitril-95% acetonitril in 0.1% trifluoroacetic acid in 7 min., flow: 2 ml/min. The desired F-18 labeled product was confirmed by co-injection with the corresponding non-radioactive F-19 fluoro-standard 2b on the analytical HPLC (t_{R}=5.1 min). The crude product was diluted with water and purified by preparative HPLC: ACE 5-C18-HL 250 mm×10 mm; isocratic, 48% acetonitrile in 0.1% trifluoroacetic acid, flow: 4 ml/min; t_{R}=29 min. The desired product was obtained as reconfirmed by co-injection with the non-radioactive F-19 fluoro standard on the analytical HPLC. The collected HPLC fraction was diluted with 40 ml water and immobilized on a Sep-Pak light 018 cartridge (Waters), which was washed with 5 ml water and eluted with 1 ml ethanol to deliver 309 MBq product (10%, corrected for decay; radiochemical purity >99%) in 1000 μl ethanol in a overall synthesis time of 90 min (compare FIGS. 13 and 14).

### Example 3

### a) Synthesis of N-(6-methoxy-1,3-benzothiazol-2-yl)-6-nitropyridine-2-carboxamide (3a)

The desired product (3a; 60 mg) was obtained from 6-nitropyridine-2-carboxylic acid (ABCR) and 207 mg of 6-methoxy-1,3-benzothiazol-2-amine according to the general procedure 1.

¹H NMR (300 MHz, CHLOROFORM-d) δ ppm 3.89 (s, 3H) 7.09 (dd, 1H) 7.34 (d, 1H) 7.77 (d, 1H) 8.36 (d, 1H) 8.43-8.58 (m, 1H) 8.61-8.77 (m, 1H)

UPLC-MS (ESI): 331 (M⁺+1, 100).

### b) Synthesis of 6-fluoro-N-(6-methoxy-1,3-benzothiazol-2-yl)pyridine-2-carboxamide (3b)

The desired product (3b; 21 mg) was obtained from 6-fluoro-pyridine-2-carboxylic acid (Aldrich) and 100 mg of 6-methoxy-1,3-benzothiazol-2-amine according to the general procedure 1.

¹H NMR (300 MHz, CHLOROFORM-d) δ ppm 3.89 (s, 3H) 7.07 (dd, 1H) 7.18-7.24 (m, 1H) 7.33 (d, 1H) 7.73 (d, 1H) 8.03-8.09 (m, 1H) 8.18-8.24 (m, 1H) 10.82 (br. s., 1H)

UPLC-MS (ESI): 304 (M⁴+1, 100).

### c) Synthesis of Synthesis of 6-[¹⁸F]-fluoro-N-(6-methoxy-1,3-benzothiazol-2-yl)pyridine-2-carboxamide (3c)

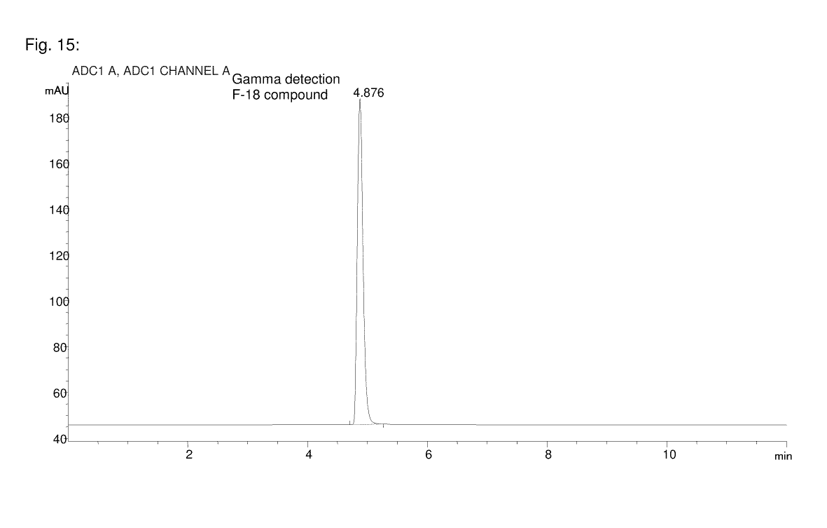

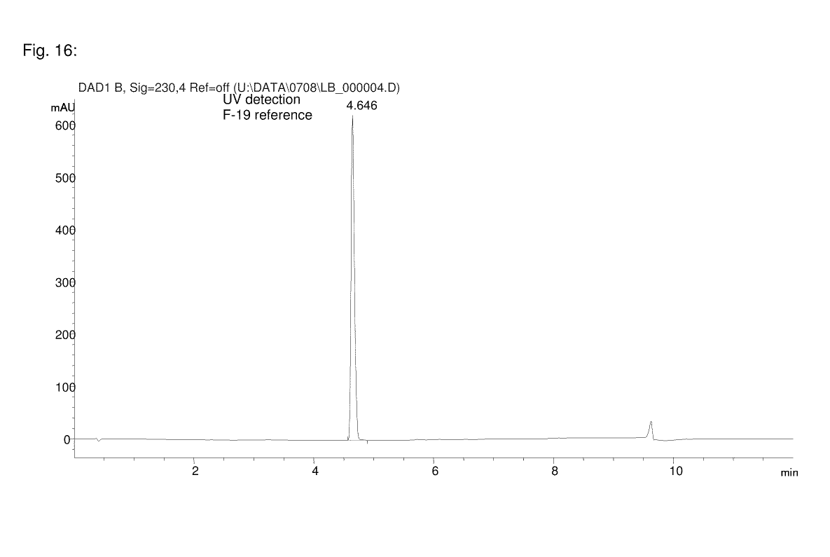

Aqueous [¹³F]Fluoride (4.8 GBq) was trapped on a QMA cartridge (Waters) and eluted with 5 mg K_{2.2.2} in 0.95 ml acetonitrile+1 mg potassium carbonate in 50 μl water into a Wheaton vial (5 ml). The solvent was removed by heating at 120° C. for 10 min under a stream of nitrogen. Anhydrous acetonitrile (1 ml) was added and evaporated as before. A solution of the NO₂-precursor 3b (5 mg) in 500 μl anhydrous DMSO was added. After heating at 180° C. for 30 min the crude reaction mixture was analyzed using analytical HPLC: ACE3-C18 50 mm×4.6 mm; solvent gradient: start 5% acetonitril-95% acetonitril in 0.1% trifluoroacetic acid in 7 min., flow: 2 ml/min. The desired F-18 labeled product was confirmed by co-injection with the corresponding non-radioactive F-19 fluoro-standard 3b on the analytical HPLC (t_{R}=4.8 min). The crude product was diluted with water and purified by preparative HPLC: ACE 5-C18-HL 250 mm×10 mm; isocratic, 45% acetonitrile in 0.1% trifluoroacetic acid, flow: 4 ml/min; t_{R}=20.5 min. The desired product was obtained (1100 MBq) as reconfirmed by co-injection with the non-radioactive F-19 fluoro standard on the analytical HPLC. The collected HPLC fraction was diluted with 40 ml water and immobilized on a Sep-Pak light C18 cartridge (Waters), which was washed with 5 ml water and eluted with 1 ml ethanol to deliver 1014 MBq product (36%, corrected for decay; radiochemical purity >99%) in 1000 μl ethanol in a overall synthesis time of 83 min (compare FIGS. 15 and 16).

### Example 4

### a) 3-bromo-4-fluoro-N-(6-methoxy-1,3-benzothiazol-2-yl)benzamide (4a)

The desired product (4; 640 mg) was obtained from 6-nitropyridine-2-carboxylic acid (ABCR) and 207 mg of 6-methoxy-1,3-benzothiazol-2-amine according to the general procedure 11.

¹H NMR (400 MHz, <DMSO>) δ ppm 3.78 (s, 3H) 7.02 (dd, 1H) 7.53 (t, 1H) 7.56 (d, 1H) 7.63 (d, 1H) 8.10-8.16 (m, 1H) 8.45 (dd, 1H) 12.85 (br. s., 1H)

UPLC-MS (ESI): 381 (M⁺+1, 100).

### Example 5

### a) Synthesis of 6-fluoro-N-(6-methoxy-1,3-benzothiazol-2-yl)pyridine-3-carboxamide (5a)

17.1 mg of the desired product 5a were obtained according to the general procedure 1 from 102 mg (0.72 mmol) 6-fluoropyridine-3-carboxylic acid (Aldrich) and 6-methoxy-1,3-benzothiazol-2-amine.

¹H NMR (400 MHz, DMSO-d₆) δ ppm 3.78 (s, 3H) 7.03 (dd, 1H) 7.35 (dd, 1H) 7.57 (d, 1H) 7.64 (d, 1H) 8.59 (td, 1H) 8.92 (d, 1H) 12.95 (hr. s., 1H)

UPLC-MS (ESI): 304 (M⁺+1, 100).

### Example 6

### a) Synthesis of 2-fluoro-N-(6-methoxy-1,3-benzothiazol-2-yl)pyridine-3-carboxamide (6a)

21 mg of the desired product 6a were obtained according to the general procedure 1 from 102 mg (0.72 mmol) 6-fluoropyridine-3-carboxylic acid (Aldrich) and 6-methoxy-1,3-benzothiazol-2-amine.

¹H NMR (300 MHz, CHLOROFORM-4) δ ppm 3.88 (s, 3H) 7.06 (dd, 1H) 7.32 (d, 1H) 7.47 (m, 1H) 7.69 (d, 1H) 8.45 (m, 1 μl) 8.70 (m, 1H) 10.05 (br. s., 1H)

UPLC-MS (ESI): 304 (M⁺+1, 100).

### Example 7

### a) Synthesis of 5-fluoro-N-(6-methoxy-1,3-benzothiazol-2-yl)pyridine-2-carboxamide (7a)

3.8 mg of the desired product 7a were obtained according to the general procedure 1 from 199 mg (1.4 mmol) 5-fluoropyridine-2-carboxylic acid (Aldrich) and 6-methoxy-1,3-benzothiazol-2-amine.

¹H NMR (300 MHz, CHLOROFORM-d) δ ppm 3.89 (s, 3H) 7.07 (dd, 1H) 7.34 (d, 1 H) 7.61-7.67 (m, 1H) 7.73 (d, 1H) 8.36 (dd, 1H) 8.51 (d, 1H) 11.01 (br. s., 1H)

UPLC-MS (ESI): 304 (M⁺+1, 100).

### Example 8

### a) Synthesis of 2-fluoro-N-(6-methoxy-1,3-benzothiazol-2-yl)pyridine-4-carboxamide (8a)

20 mg of the desired product 7a were obtained according to the general procedure 1 from 102 mg (0.72 mmol) 2-fluoropyridine-4-carboxylic acid (Chempur) and 6-methoxy-1,3-benzothiazol-2-amine.

¹H NMR (300 MHz, CHLOROFORM-d) δ ppm 3.89 (s, 3H) 7.04 (dd, 1H) 7.32 (d, 1H) 7.49 (d, 1H) 7.59 (s, 1H) 7.80 (d, 1H) 8.27 (s, 1H) 8.42 (d, 1H)

UPLC-MS (ESI): 304 (M⁺+1, 100).

### Example 9

### a) Synthesis of 2-[(phenylcarbonyl)amino]-1,3-benzothiazol-6-yl benzoate (9a)

The desired product 9a was obtained in 770 mg yield from 500 mg (3 mmol) 2-amino-1,3-benzothiazol-6-ol (ABCR) and according to the general procedure 11 but with 3 eq. instead of 1.5 eq. benzyl chloride.

¹H NMR (400 MHz, <CDCl13>) δ ppm 7.22-7.26 (m, 1H) 7.48-7.58 (m, 5H) 7.59-7.70 (m, 3H) 7.75 (d, 1H) 7.99-8.05 (m, 2H) 8.22-8.26 (m, 2H) 10.55 (hr. s., 1H)

UPLC-MS (ESI): 375 (M⁺+1, 100).

### b) Synthesis of N-(6-hydroxy-1,3-benzothiazol-2-yl)benzamide (9b)

To a stirred solution of 720 mg (1.92 mmol) 9a in 70 ml methanol was added 670 mg powdered sodium hydroxide. The reaction mixture was stirred for 2.5 h. The volume of the reaction mixture was reduced and water and diluted hydrochloric acid (aq.) were added so that the pH value was adjusted to 3. The aqueous phase was extracted three times with dichloromethane:iso-propanol mixture. The combined organic phases were washed with brine, dried with magnesium sulphate and reduced in vacuum. The crude product was purified by RP-HPLC. The desired product 9b was obtained as oil with 340 mg.

UPLC-MS (ESI): 271 (M⁺+1, 100).

¹H NMR (400 MHz, <DMSO>) δ ppm 6.88 (dd, 1H) 7.28 (d, 1H) 7.48-7.58 (m, 3H) 7.58-7.65 (m, 1H) 8.04-8.10 (m, 2H)

### c) Synthesis of N-[6-(3-fluoropropoxy)-1,3-benzothiazol-2-yl]benzamide (9c)

The desired product 9c was obtained from 163 mg (0.6 mmol) 9b and fluoro-propylbromide according general procedure 4 in 44% yield (88 mg).

¹H NMR (400 MHz, <CDCl3>) δ ppm 2.32 (m, 1H) 2.36-2.42 (m, 1H) 4.51 (t, 1H) 4.59-4.68 (m, 3H) 7.04 (dd, 1H) 7.31 (d, 1H) 7.37 (m, 1H) 7.43-7.56 (m, 3H) 8.32-8.38 (m, 2H)

UPLC-MS (ESI): 331 (M⁺+1, 100).

### Example 10

### a) Synthesis of 4-methoxy-N-(6-methyl-1,3-benzothiazol-2-yl)benzamide (10a)

The desired product 10a (120 mg) was obtained from 0.72 mmol 6-methyl-1,3-benzothiazol-2-amine (Aldrich) and p-methoxy benzylchloride (Aldrich) according to general procedure 11

¹H NMR (300 MHz, <DMSO>) δ ppm 2.39 (s, 3H) 3.82 (s, 3H) 7.05 (m, 2H) 7.23 (dd, 1H) 7.61 (d, 1H) 7.75 (s, 1H) 8.10 (m, 2H) 12.60 (br. s., 1H)

UPLC-MS (ESI): 299 (M⁺+1, 100).

### b) Synthesis of 4-hydroxy-N-(6-methyl-1,3-benzothiazol-2-yl)benzamide (10b)

To a stirred solution of 73 mg (0.24 mmol) 10a in 7 ml glacial acid 7 ml aqueous hydrobromic acid-solution (48%) was added. The mixture was stirred for 30 min at reflux. The solution was reduced in vacuum and water was added. The aqueous phase was extracted with dichloromethane/iso-propanol (10:1). The combined organic phases were washed with brine, dried with magnesium sulphate and reduced in vacuum. The crude product was purified by RP-HPLC. The desired product 10b was obtained as solid (21 mg).

UPLC-MS (ESI): 285 (M⁺+1, 100).

### c) Synthesis of 4-(2-fluoroethoxy)-N-(6-methyl-1,3-benzothiazol-2-yl)benzamide (10c)

The desired product 10c (5 mg) was obtained from 10b (20 mg) and fluoro ethyl bromide according to the general procedure 4.

¹H NMR (300 MHz, DMSO-d₆) δ ppm 2.36 (s, 3H) 4.20-4.38 (m, 1H) 4.64-4.98 (m, 3H) 6.82 (d, 1H) 7.04 (d, 1H) 7.26-7.33 (m, 1H) 7.57 (t, 1H) 7.66 (d, 1H) 8.07 (d, 1H) 8.17 (d, 1H)

UPLC-MS (ESI): 331 (M⁺+1, 100).

### Example 11

### a) Synthesis of N-(6-{2-[2-(2-fluoroethoxy)ethoxy]ethoxy}-1,3-benzothiazol-2-yl)benzamide (11a)

The desired product 11a (18 mg) were obtained from 55 mg (0.18 mg) 2-[2-(2-fluoroethoxy)ethoxy]ethyl 4-methylbenzenesulfonate (which can be prepared from 2-[2-(2-hydroxyethoxy)ethoxy]ethyl 4-methylbenzenesulfonate (Journal of Medicinal Chemistry; English; 50; 9; 2007; 2157-2165) according to the general procedure 13 (purification by silica column chromatography) and 9b (49 mg) according to the general procedure 4.

UPLC-MS (ESI): 405 (M⁺+1, 100).

### Example 12

### a) Synthesis of tert-butyl[6-(2-fluoroethoxy)-1,3-benzothiazol-2-yl]carbamate (12a)

The desired product (14.9 mg) was obtained from 37 mg tert-butyl (6-hydroxy-1,3-benzothiazol-2-yl)carbamate (US2007193488) and commercially available 2-fluoro-ethyl iodide according to the general procedure 4.

¹H NMR (300 MHz, <CDCl3>) δ ppm 1.60 (s, 9H) 4.44-4.52 (m, 1H) 4.56 (t, 1H) 4.65 (t, 1H) 4.80 (t, 1H) 6.90 (dd, 1H) 7.20 (d, 1H) 7.60 (d, 1H)

UPLC-MS (ESI): 313 (M⁺+1, 100).

### b) Synthesis of 6-(2-fluoroethoxy)-1,3-benzothiazol-2-amine (12b)

The desired product 12b (˜40 mg) was obtained from 12a (60 mg) according to the general procedure 14. The desired crude product was not purified for the next reaction.

¹H NMR (300 MHz, <MeOD>) δ ppm 3.76-3.83 (m, 1H) 3.89 (t, 1H) 4.57-4.67 (m, 1H) 4.75-4.84 (m, 1H) 6.95 (dd, 1H) 7.19 (d, 1H) 7.36 (d, 1H)

UPLC-MS (ESI): 313 (M⁺+1, 100).

### c) Synthesis of 4-fluoro-N-[6-(2-fluoroethoxy)-1,3-benzothiazol-2-yl]benzamide (12c)

The desired product (12c) (21 mg) was obtained from 12b (73 mg, 0.34 mmol) and 4-Fluoro-benzoic acid according to the general procedure 1.

¹H NMR (400 MHz, <DMSO>) δ ppm 3.63 (q, 1H) 3.70 (q, 1H) 4.53 (t, 1H) 4.64 (t, 1H) 7.09 (dd, 1H) 7.35-7.47 (m, 2H) 7.62 (d, 1H) 8.16 (dd, 2H) 8.28-8.34 (m, 1H)

UPLC-MS (ESI): 335 (M⁺+1, 100).

### Example 13

### a) 3-cyano-4-fluoro-N-(6-methoxy-1,3-benzothiazol-2-yl)benzamide (13a)

The desired product (13a; 25 mg) was obtained from 4-fluoro-3-(trifluoromethyl)benzoic acid (Apollo) and 200 mg of 6-methoxy-1,3-benzothiazol-2-amine according to the general procedure 1.

UPLC-MS (ESI): 328 (M⁺+1, 100).

### Example 14

### a) Synthesis of 4-fluoro-N-(6-methoxy-1,3-benzothiazol-2-yl)-3-(trifluoromethyl)benzamide (14a)

The desired product (14a; 22 mg) was obtained from 4-fluoro-3-(trifluoromethyl)benzoic acid (Aldrich) and 200 mg of 6-methoxy-1,3-benzothiazol-2-amine according to the general procedure 1.

UPLC-MS (ESI): 371 (M⁺+1, 100).

### ABBREVIATIONS AND ACRONYMS

A comprehensive list of the abbreviations used by organic chemists of ordinary skill in the art appears in The ACS Style Guide (third edition) or the Guidelines for Authors for the Journal of Organic Chemistry. The abbreviations contained in said lists, and all abbreviations utilized by organic chemists of ordinary skill in the art are hereby incorporated by reference. For purposes of this invention, the chemical elements are identified in accordance with the Periodic Table of the Elements, CAS version, Handbook of Chemistry and Physics, 67th Ed., 1986-87.

More specifically, when the following abbreviations are used throughout this disclosure, they have the following meanings:

Boc tert-butoxycarbonyl
DAST diethylaminosulfur trifluoride
ESI electro spray ionisation
eq.; equiv. equivalent
h hour, hours
m-CPBA meta-chloroperbenzoic acid
TFA trifluoroacetic acid
UPLC-MS Ultra Performance Liquid Chromatography-mass spectrometry

1. A compound of formula I 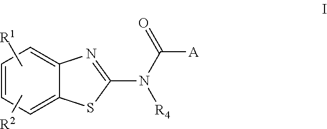
wherein
A is selected from the group comprising 1-(N—R⁹)-2,3-dihydro-1H-indol-5-yl, 1-(N—R⁹)-1H-indol-5-yl, phenyl and pyridyl, whereas A is substituted with R⁵ and R⁶.
R¹ and R² are independently and individually, at each occurrence, selected from the group comprising hydrogen, halo, cyano, trifluoromethyl, (C₁-C₅)alkyl, (C₂-C₅)alkynyl, (C₂-C₅)alkenyl, (C₁-C₅)alkoxy, (R⁷)O—, L-(CH₂—CH₂—O)ₙ—, L, L-(C₁-C₆)alkoxy, (C₁-C₅)sulfanyl and L-(C₁-C₅)sulfanyl;
R⁴ is selected from the group comprising hydrogen and (C₁-C₄)alkyl;
R⁵ and R⁶ are independently and individually, at each occurrence, selected from the group comprising hydrogen, L, L-(C₁-C₅)alkyl, L-(C₂-C₅)alkenyl, L-(C₁-C₅)alkoxy, L-(C₂-C₅)alkynyl, (C₁-C₅)sulfanyl, L-(C₁-C₅)sulfanyl, (C₁-C₅)alkyl, (C₂-C₅)alkenyl, (C₁-C₅)alkoxy, (R⁷)O—, halo, trifluoromethyl, cyano, —C(O)O—((C₁-C₅)alkyl), —N(R⁸)(L-(C₁-C₅)alkyl), —N(L-(C₁-C₄)alkyl)((C₁-C₄)alkyl), —N(R⁵)((C₁-C₄)alkyl) and —N((C₁-C₄)alkyl)₂;
L is selected from the group comprising R¹⁰, R³, F, [¹⁹F]fluoro and [¹⁸F]fluoro;
R³ is a leaving group;
R¹⁰ is selected from the group comprising R²⁰ and R³⁰;
R²⁰ is selected from the group comprising iodo, —Sn((C₁-C₆)alkyl)₃, —B(OR⁶⁰)(OR⁶¹) and NMe₂;
R³⁰ is hydroxy;
R⁷ is selected from the group comprising hydrogen and R¹⁷;
R⁸ is selected from the group comprising hydrogen and R¹⁸;
wherein n is an integer from 2 to 6;
including all isomeric forms of said compound, including but not limited to enantiomers and diastereoisomers as well as racemic mixtures,
and any pharmaceutically acceptable salt, ester, amide, complex or prodrug thereof;
with the proviso that compounds of Formula I contain exactly one L.

2. A compound of claim 1, wherein
A is selected from the group comprising phenyl and pyrid-2-yl, whereas A is substituted with R⁵ and R⁶;
R¹ and R² are independently and individually, at each occurrence, selected from the group comprising hydrogen, fluoro, lode, L, (C₁-C₃)alkyl and (C₁-C₃)alkoxy;
R⁴ is selected from the group comprising hydrogen and methyl;
R⁵ and R⁶ are independently and individually, at each occurrence, selected from the group comprising hydrogen, L, L-(C₁-C₃)alkoxy, methyl, bromo, fluoro, trifluoromethyl, cyano, —N(R⁸)(methyl) and —N(methyl)₂;
L is selected from the group consisting of [¹⁸F]fluoro, [¹⁹F]fluoro, or a leaving group.

3. A compound according to claim 1 selected from the group consisting of compounds having the formula 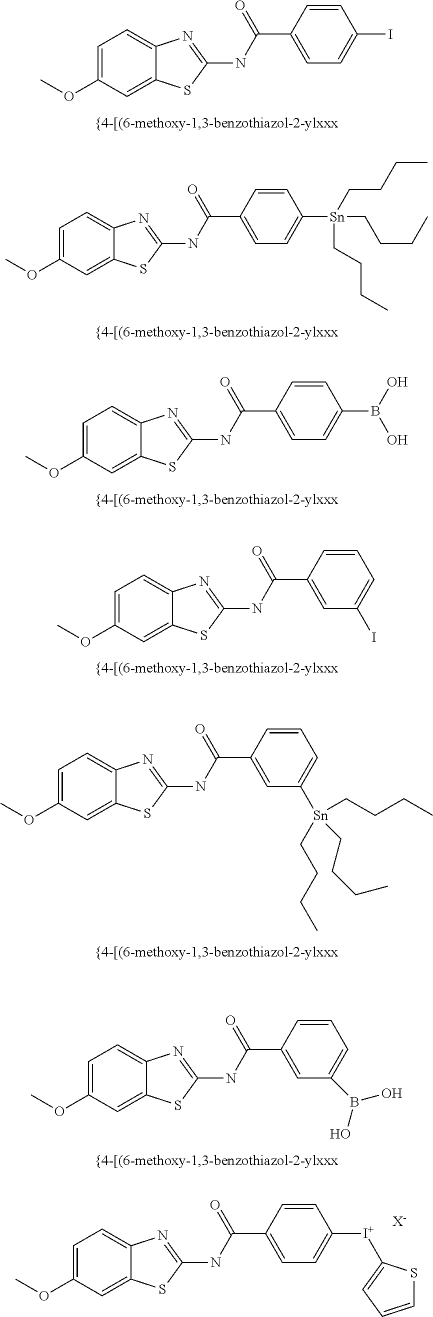 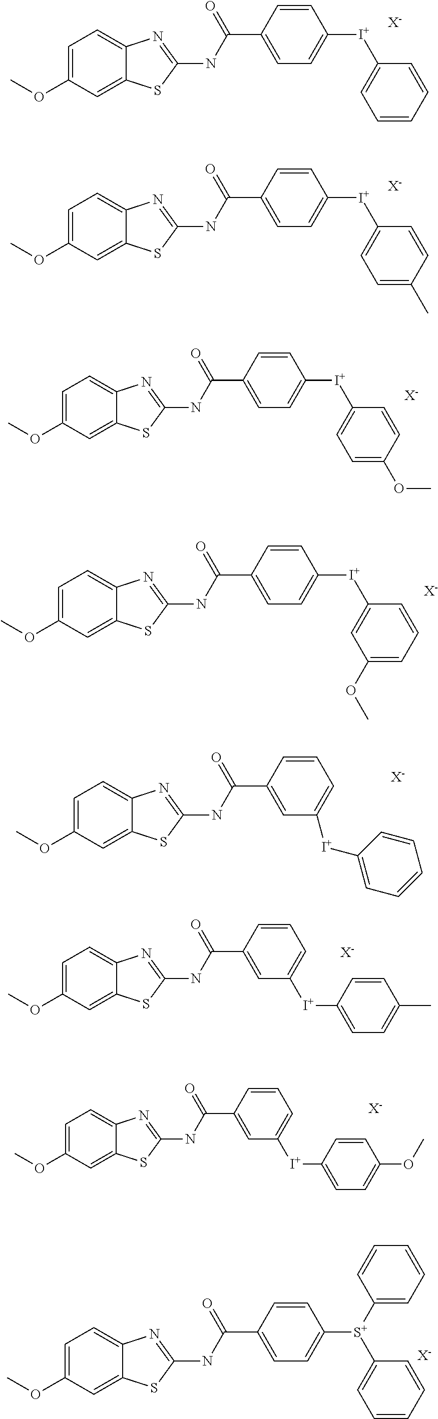 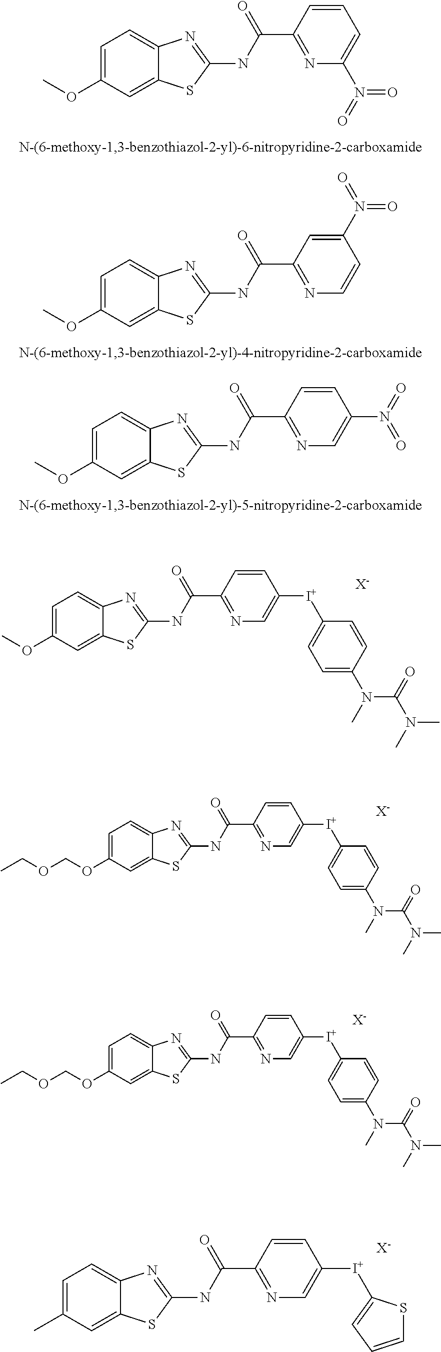 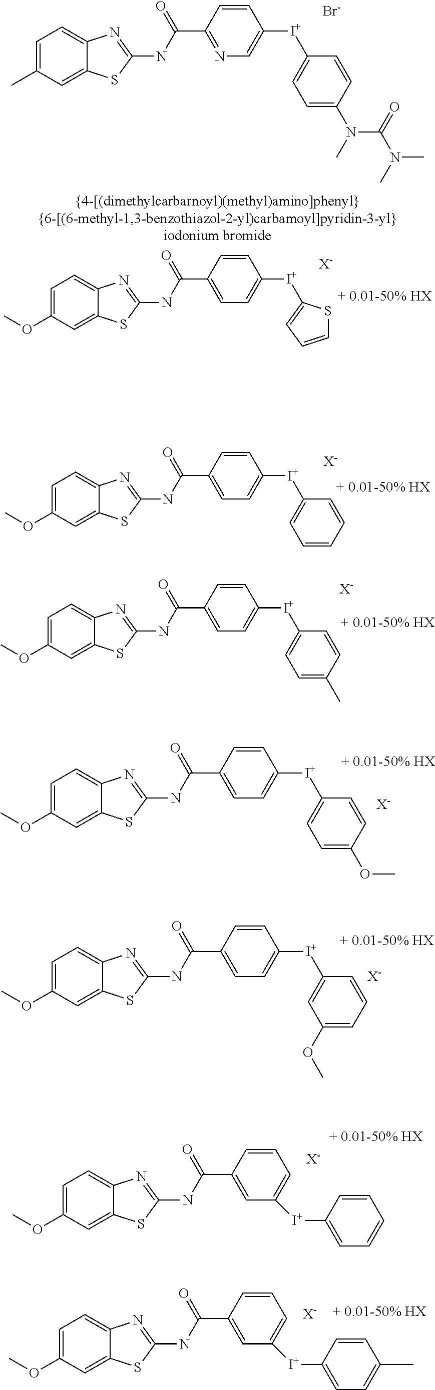 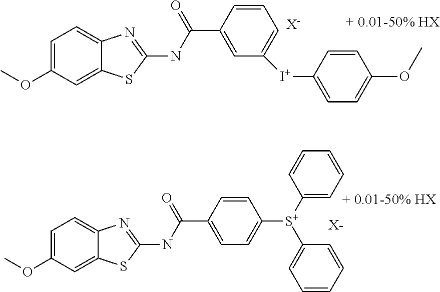
wherein X⁻ is selected from the group comprising anion of an inorganic acid and anion of an organic acid.

4. A compound according to claim 1 selected from the group consisting of compounds having the formula

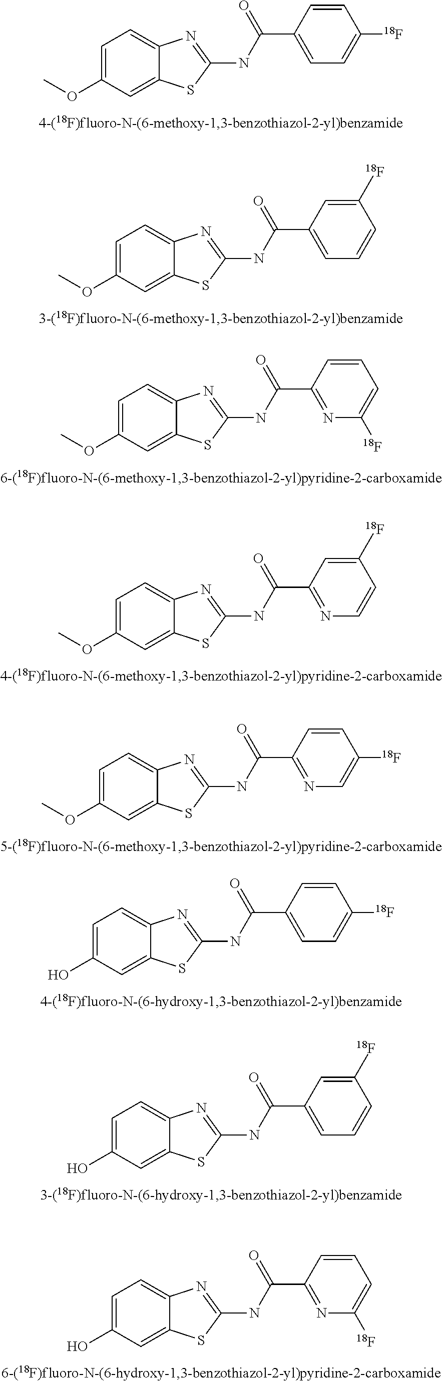

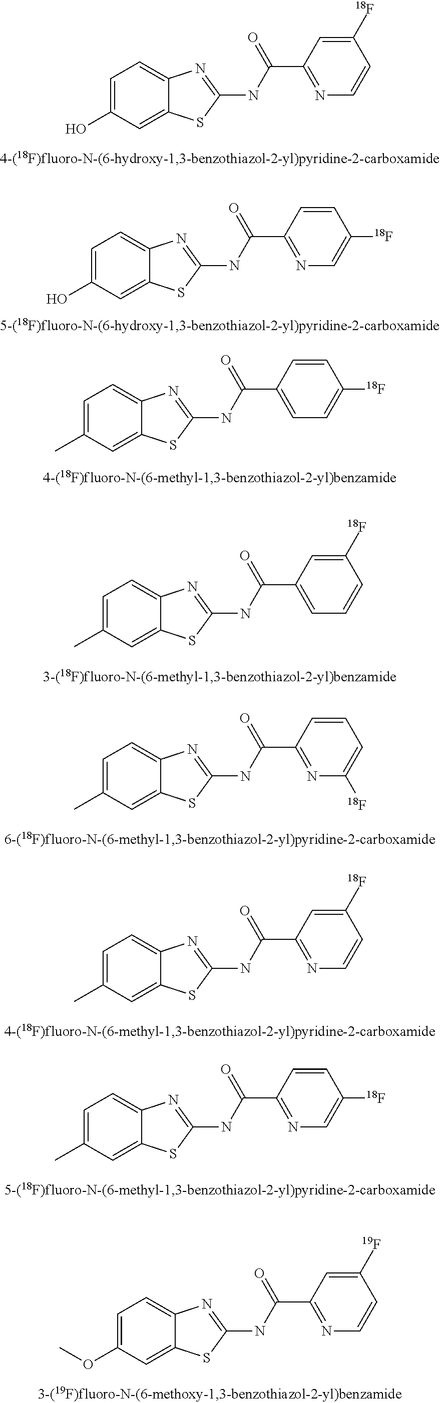

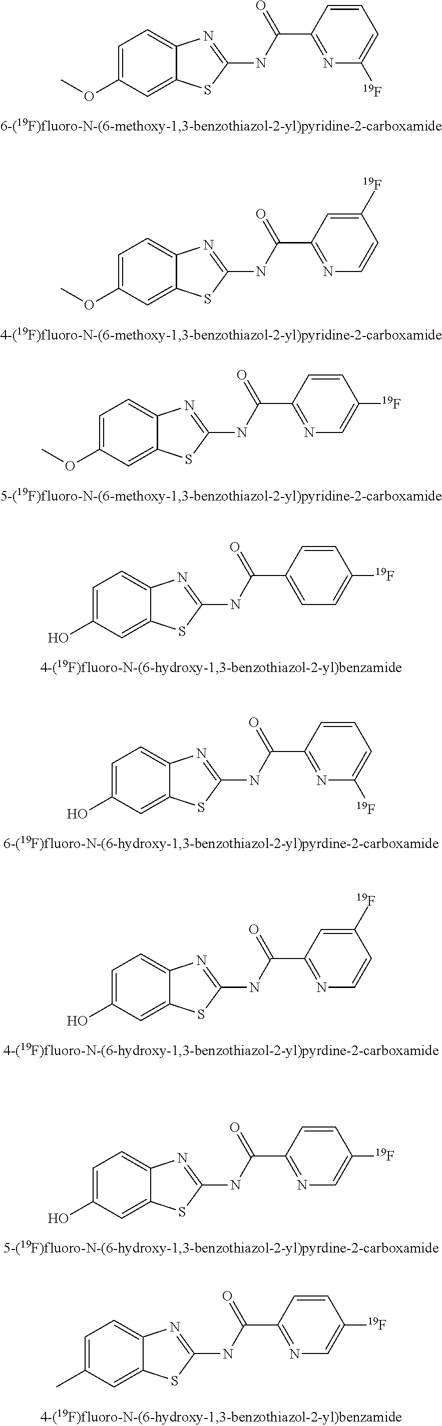

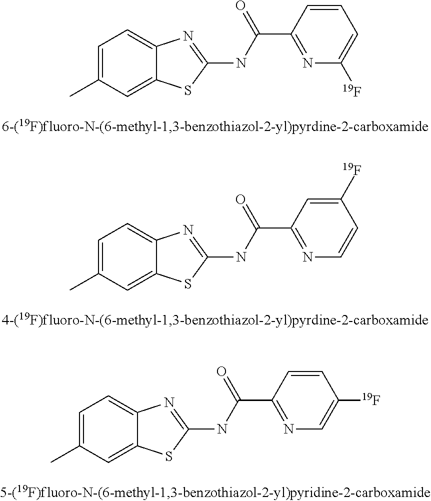

5. A compound according to claim 4 selected from the group consisting of

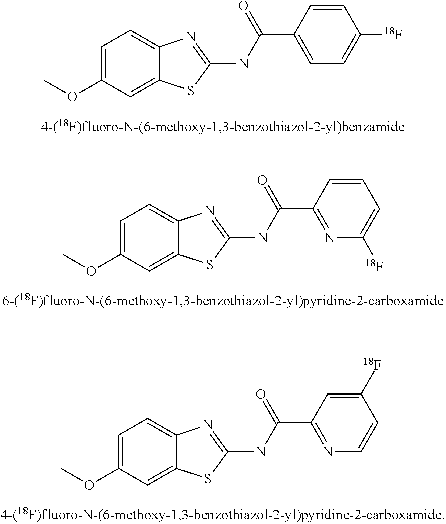

6. A radioactively labelled halogenated compound according to claim 1 as a compound for diagnostic imaging.

7. A compound according to claim 6, wherein the radioactive label is [F-18].

8. A compound according to claim 6 as a compound for diagnostic imaging of a disease selected from the group consisting of Alzheimer's disease, a neurodegenerative disorder, or an amyloidosis.

9. A method for the preparation of a fluorinated compound according to claim 1 the method comprising reacting a suitable precursor molecule with a fluorinating agent.

10. A method for the preparation of a fluorinated compound, the method comprising reacting a respective precursor molecule of claim 3 with a fluorinating agent.

11. A method for diagnosing a disease in a mammal selected form the group consisting of Alzheimer's disease, a neurodegenerative disorder, or an amyloidosis, the method comprising administering a radioactively labelled compound of claim 1 to said mammal, imaging said mammal and detecting the signal.

12. The method according to claim 11, wherein the compound is a [¹⁸F] labelled compound of claims 4.

13. The method of claim 12, wherein said imaging is performed using a method selected from the group consisting of PET, SPECT, MR-spectroscopy, and MR-tomography.

14. A method according to claim 11, wherein the effect of a therapy is monitored.

15. A method of imaging amyloid plaques in a mammal, said method comprising administering a radioactively labelled compound of claim 1 to said mammal, imaging said mammal and detecting the signal.

16. A compound of formula VI 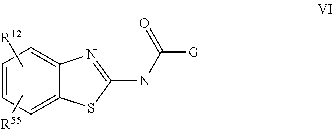
wherein
G is selected from the group comprising 1-(N—R¹¹)-2,3-dihydro-1H-indol-5-yl, 1-(N—R¹¹)-1H-indol-5-yl, phenyl and pyridyl, whereas G is substituted with R¹³ and R¹⁵.
R¹¹ is selected from the group comprising (C₁-C₄)alkyl, R¹⁸ and R¹⁴;
R¹² is selected from the group comprising hydrogen and R¹⁴—O—
R¹³ is selected from the group comprising hydrogen, (R¹⁴)O— and —N((C₁-C₄)alkyl)R¹⁴;
R¹⁴ is hydrogen:
R¹⁵ and R⁵⁵ are independently and individually selected from the group comprising hydrogen, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
R¹⁸ is a amine-protecting group;
including all isomeric forms of said compound, including but not limited to enantiomers and diastereoisomers as well as racemic mixtures,
and any pharmaceutically acceptable salt, ester, amide, complex or prodrug thereof;
with the proviso that compounds of formula IV contain exactly one R¹⁴.

17. A method of preparation of compounds of Formula Ib, 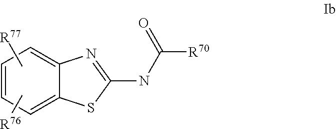
said method comprising the steps:
F-fluorinating a compound of formula V 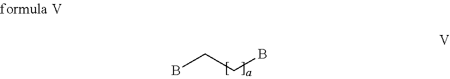
with an F-fluorinating agent to yield a compound of formula IV, 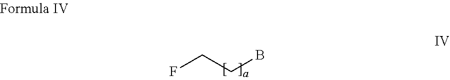
substituting said compound of formula IV with a compound of formula VI 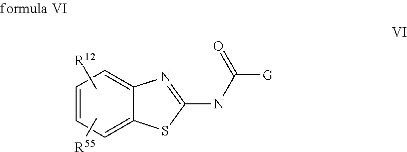
deprotection in those cases where compounds of formula VI comprise R¹⁸ or R¹⁷;
wherein
R⁷⁰ is selected from the group comprising 1-(N—R⁷¹)-2,3-dihydro-1H-indol-5-yl, 1-(N—R⁷¹)-1H-indol-5-yl, phenyl and pyridyl, whereas R⁷⁰ is substituted with R⁷³ and R⁷⁵;
R⁷¹ is selected from the group comprising (C₁-C₄)alkyl, hydrogen, R¹⁸ and (L-CH₂—(CH₂)ₐ)—;
R⁷³ is selected from the group comprising hydrogen, (L-CH₂—(CH₂)ₐ—)O—, —N(L-CH₂—(CH₂)ₐ—)(H) and —N((C₁-C₄)alkyl)(L-CH₂—(CH₂)ₐ—);
R⁷⁵ and R⁷⁶ are independently and individually selected from the group comprising hydrogen, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
R⁷⁷ is selected from the group comprising hydrogen and (L-CH₂—(CH₂)ₐ)—O;
wherein L in Formula Ib is [¹⁸F]fluoro or [¹⁹F]fluoro, with the proviso that compounds of Formula Ib comprise exactly one L;
F in Formula IV is [¹⁸F]fluoro or [¹⁹F]fluoro;
a is an integer from 0 to 5;
B is a leaving group;
G is selected from the group comprising 1-(N—R¹¹)-2,3-dihydro-1H-indol-5-yl, 1-(N—R¹¹)-1H—I;dol-5-yl, phenyl and pyridyl, whereas G is substituted with R¹³ and R¹⁵;
R¹¹ is selected from the group comprising (C₁-C₄)alkyl, R¹⁸ and R¹⁴;
R¹² is selected from the group comprising hydrogen and (R¹⁴)O—;
R¹³ is selected from the group comprising hydrogen, (R¹⁴)O—, —N(R¹⁴)(R¹⁸) and —N((C₁-C₄)alkyl)(R¹⁴);
R¹⁴ is hydrogen;
R¹⁵ and R⁵⁵ are independently and individually selected from the group comprising hydrogen, (R¹⁷)O—, halo, cyano, trifluoromethyl, (C₁-C₅)alkyl, (C₂-C₅)alkynyl, (C₁-C₅)sulfanyl, (C₂-C₅)alkenyl and (C₁-C₅)alkoxy;
R¹⁷ is a phenol protecting group;
R¹⁸ is a amine-protecting group;
including all isomeric forms of said compound, including but not limited to enantiomers and diastereoisomers as well as racemic mixtures,
and any pharmaceutically acceptable salt, ester, amide, complex or prodrug thereof;
wherein said F-fluorinating agent is as defined above,
and wherein F=¹⁸F or ¹⁹F,
with the proviso that compounds of formula VI contain exactly one R¹⁴.

18. A method of preparation of compounds of Formula Ic, 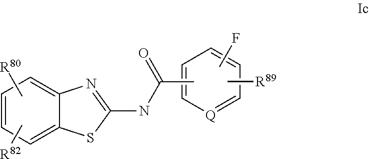
comprises the step:
F-fluorinating a compound of formula XV 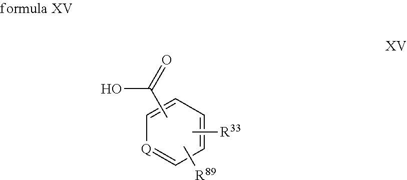
with an F-fluorinating agent to yield a compound of formula XIV, 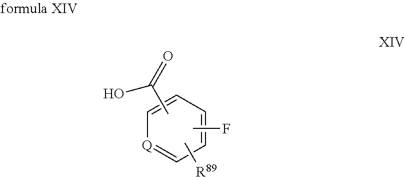
coupling said compound of formula XIV (or an activated derivative (e.g. active ester) of said compound of Formula XIV) with a compound of formula XVI 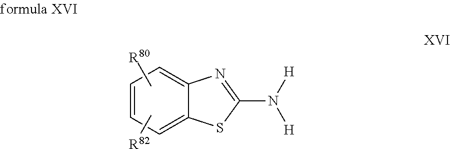
wherein F in Formula XIV and in Formula Ic is selected from the group comprising [¹⁸F]fluoro and [¹⁹F]fluoro;
Q is selected from the group comprising nitrogen and C(H);
R³³ is selected from the group comprising —I⁺(R²⁵)(X⁻), —I⁺(R²⁶)(X⁻), nitro, —N⁺(Me)₃(X⁻), —S⁺(R²⁵)(R²⁵)(X⁻), —S⁺(R²⁵)(R²⁶)(X⁻), —S⁺(R²⁶)(R²⁶)(X⁻), chloro and bromo;
R⁸⁹ is selected from the group comprising hydrogen, (C₁-C₅)alkyl, (C₂-C₅)alkenyl, (C₁-C₅)alkoxy, halo, trifluoromethyl, cyano, —C(O)O—((C₁-C₅)alkyl), —N(R¹⁸)((C₁-C₄)alkyl) and —N((C₁-C₄)alkyl)₂;
R¹⁸ is a amine-protecting group;
R⁸⁰ and R⁸² are independently and individually, at each occurrence, selected from the group comprising hydrogen, halo, cyano, trifluoromethyl, (C₁-C₅)alkyl, (C₂-C₅)alkynyl, (C₂-C₅)alkenyl, (C₁-C₅)alkoxy and (R¹⁷)O—;
R¹⁷ is a phenol protecting group;
X⁻ is selected from the group comprising anion of an inorganic acid and anion of an organic acid;
R²⁵ is aryl and
R²⁶ is heteroaryl.

19. A kit, comprising a compound according to claim 1.
